SEQUENCE LISTING (4 sequences)

SEQ ID NO: 1 - DNA, 1521 bp, Homo sapiens
  CDS (1)..(1521)
atggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacg

SEQ ID NO: 2 - protein, 507 aa, Homo sapiens
MASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 3 - DNA, 1080 bp, Homo sapiens
  CDS (1)..(1080)
atcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaacttta

SEQ ID NO: 4 - protein, 360 aa, Homo sapiens
IPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTL

(19) United States
(12) Patent Application Publication
(10) Pub. No.: US 2007/0015270 A1
(43) Pub. Date: Jan. 18, 2007

(54) Crystalline PDE4D2 catalytic domain complex, and methods for making and employing same

(75) Inventors: Hengming Ke, Chapel Hill, NC (US); Qing Huai, Carrboro, NC (US)

Correspondence Address: JENKINS & WILSON, PA, 3100 TOWER BLVD, SUITE 1400, DURHAM, NC, 27707, US

(73) Assignee: The University of North Carolina at Chapel Hill

(21) Appl. No.: 10/771,186

(22) Filed: Feb. 3, 2004

Related U.S. Application Data
(60) Provisional application No. 60/444,610, filed on Feb. 3, 2003.

(51) Int. Cl. C12N 9/16

(52) U.S. Cl. 435/196

(57) ABSTRACT

The presently disclosed subject matter provides a crystalline form of a substantially pure phosphodiesterase 4D2 (PDE4D2) polypeptide. Also provided is a crystalline form of a substantially pure phosphodiesterase 4D2 (PDE4D2) polypeptide in complex with a ligand. Also provided are methods for generating the crystalline forms of the presently disclosed subject matter and methods for identifying and designing phosphodiesterase ligands and modulators. Also provided are scalable three-dimensional configurations of points and computer readable storage media containing digitally encoded structural data.

### CROSS-REFERENCE TO RELATED APPLICATION

This application is based on and claims priority to U.S. Provisional Patent Application Ser. No. 60/444,640, filed Feb. 3, 2003, herein incorporated by reference in its entirety.

### GRANT STATEMENT

This work was supported by grant GM59791 from the U.S. National Institutes of Health (NIH). Thus, the U.S. government has certain rights in the presently disclosed subject matter.

### TECHNICAL FIELD

The presently disclosed subject matter relates generally to the structures of the PDE4D2 catalytic domain, and more particularly to crystal structures of an unliganded PDE4D2 catalytic domain and a PDE4D2 catalytic domain in complex with a ligand. The presently disclosed subject matter also relates to PDE4D2 catalytic domain binding compounds and to the design of compounds that bind to the PDE4D2 catalytic domain.

### Abbreviations

A—Ångstrom
AMP—adenosine monophosphate
ATCC—American Type Culture Collection
cAMP—cyclic 3′,5′ adenosine monophosphate
CaMV—cauliflower mosaic virus
CCDC—Cambridge Crystallographic Data Center
CD—catalytic domain
cDNA—complementary DNA
cGMP—cyclic 3′,5′ guanosine monophosphate
CNS—Crystallography and NMR System
CPU—central processing unit
CRT—cathode ray tube
DMSO—dimethyl sulfoxide
DNA—deoxyribonucleic acid
EST—expressed sequence tag
FEDs—field emission displays
GMP—guanosine monophosphate
HEPES—N-2-hydroxyethylpiperazine-N′-2-ethanesulfonic acid
K_{d}—dissociation constant
kD—kilodalton(s)
LCDs—liquid crystal displays
LED—light emitting diode
MIR—multiple isomorphous replacement
MPD—methyl pentanediol
nt—nucleotide(s)
PCR—polymerase chain reaction
PDE—phosphodiesterase
PDE4D2—phosphodiesterase 4D2
PEG—polyethylene glycol
pl—isoelectric point
RAM—random access memory
RUBISCO—ribulose bisphosphate carboxylase
SIRAS—single isomorphous replacement with anomalous scattering
TMV—tobacco mosaic virus

Amino Acid Abbreviations, Codes, and Functionally Equivalent Codons

| | 3- | 1- | |
|---|---|---|---|
| Amino Acid | Letter | Letter | Codons |
| Alanine | Ala | A | GCA GCC GCG GCU |
| Arginine | Arg | R | AGA AGG CGA CGC CGG CGU |
| Asparagine | Asn | N | AAC AAU |
| Aspartic | Asp | D | GAC GAU |
| Acid | | | |
| Cysteine | Cys | C | UGC UGU |
| Glutamic | Glu | E | GAA GAG |
| acid | | | |
| Glutamine | Gln | Q | CAA CAG |
| Glycine | Gly | G | GGA GGC GGG GGU |
| Histidine | His | H | CAC CAU |
| Isoleucine | Ile | I | AUA AUC AUU |
| Leucine | Leu | L | UUA UUG CUA CUC CUG CUU |
| Lysine | Lys | K | AAA AAG |
| Methionine | Met | M | AUG |
| Phenyl- | Phe | F | UUC UUU |
| alanine | | | |
| Proline | Pro | P | CCA CCC CCG CCU |
| Serine | Ser | S | ACG AGU UCA UCC UCG UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |

### BACKGROUND ART

Cyclic 3′,5′-adenosine and guanosine monophosphates (cAMP and cGMP, respectively) are intracellular second messengers that mediate the response of cells to a wide variety of stimuli, primarily through the activation of cyclic nucleotide activated protein kinases. Regulation of cAMP and cGMP concentrations in vivo is essential for many metabolic processes, such as cardiac and smooth muscle contraction, glycogenolysis, platelet aggregation, secretion, lipolysis, ion channel conductance, apoptosis, growth control, and neurological function (reviewed by Houslay 1998; Antoni, 2000; Lucas et al., 2000; Klein, 2002; Stork and Schimitt, 2002; Mehats et al., 2002). Cyclic nucleotide phosphodiesterases (PDEs) are enzymes hydrolyzing cAMP and/or cGMP to adenosine monophosphate (AMP) and/or guanosine monophosphate (GMP) and are essential for the regulation of cyclic nucleotide concentrations in the cell (Torphy, 1998; Conti and Jin, 1999; Soderling and Beavo, 2000).

The human genome encodes twenty-one PDE genes categorized into 11 families (Thompson, 1991; Manganiello et al., 1995; Müller et al., 1996; Houslay and Milligan, 1997; Zhao et al., 1997; Houslay et al., 1998; Torphy 1998; Corbin and Francis, 1999; Soderling and Beavo, 2000; Francis et al, 2001; Mehats et al., 2002;). Additional diversity is generated through the alternate splicing of PDE mRNAs, producing over 60 PDE isoforms in various human tissues. Family-selective inhibitors of PDEs constitute a rapidly growing class of pharmaceuticals directed against several diseases and are widely studied as cardiotonic agents, vasodilators, smooth muscle relaxants, anti-depressants, anti-thrombotic compounds, anti-asthma compounds, and agents for improving cognitive functions such as memory (Corbin and Francis, 2002; Giembycz, 2000, 2002; Huang et al., 2001; Reilly and Mohler, 2001; Rotella, 2002; Souness et al., 2000; Spina, 2003). For example, the PDE5 inhibitor sildenafil (VIAGRA®) is a drug for male erectile dysfunction and the PDE3 inhibitor cilostamide is a drug for heart diseases. Selective inhibitors of PDE4 form the largest group of inhibitors for any PDE family, and have been studied as anti-inflammatory drugs targeting asthma and chronic obstructive pulmonary disease (Piaz and Giovannoni, 2000; Barnette and Underwood, 2000; Giembycz, 2002; Sturton and Fitzgerald, 2002).

All PDE enzymes share 25% sequence homology throughout a conserved catalytic domain of approximately 300 amino acids, suggesting that diverse PDE enzymes share a conserved active site structure and enzymatic mechanism. However, each PDE family recognizes a specific substrate and possesses its own selective inhibitors. The families PDE4, 7, and 8 prefer to hydrolyze cAMP while PDE5, 6, and 9 are cGMP specific. PDE1, 2, 3, 10, and 11 can hydrolyze both cAMP and cGMP.

To date, it is not known how the similar catalytic pockets of the different PDE families distinguish cAMP from cGMP and what the mechanism of hydrolysis is. The presently disclosed subject matter addresses these and other needs in the art.

### SUMMARY

The presently disclosed subject matter provides a crystalline form comprising a substantially pure phosphodiesterase 4D2 (PDE4D2) polypeptide. In one embodiment, the presently disclosed subject matter provides a crystalline form comprising a substantially pure phosphodiesterase 4D2 (PDE4D2) catalytic domain polypeptide in complex with a ligand. In one embodiment, the crystalline form has unit cell a=99.2 Å; b=111.2 Å; c=159.7 Å. In another embodiment, the crystalline form has a space group of P2₁2₁2₁. In another embodiment, the crystalline form comprises four phosphodiesterase 4D2 (PDE4D2) catalytic domain polypeptides. In another embodiment, the crystalline form is such that the three-dimensional structure of the crystallized complex can be determined to a resolution of about 2.3 Å or better. In another embodiment of the presently disclosed subject matter, the phosphodiesterase 4D2 (PDE4D2) catalytic domain polypeptide has the amino acid sequence shown in SEQ ID NO: 4. In yet another embodiment, the complex has a crystalline structure further characterized by the coordinates corresponding to one of Table 4 and Table 5.

The presently disclosed subject matter also provides methods of generating a crystalline form comprising a phosphodiesterase 4D2 (PDE4D2) catalytic domain polypeptide in complex with a ligand, the method comprising: (a) incubating a solution comprising a phosphodiesterase 4D2 (PDE4D2) catalytic domain and a ligand; and (b) crystallizing the phosphodiesterase 4D2 (PDE4D2) catalytic domain polypeptide and ligand by vapor diffusion, whereby a crystalline form of a phosphodiesterase 4D2 (PDE4D2) catalytic domain polypeptide in complex with a ligand is generated. In another embodiment, the crystalline form is grown by vapor diffusion against a well buffer comprising 50 mM HEPES (pH 7.5), 15% PEG3350, 25% ethylene glycol, 5% methanol, and 5% DMSO. In another embodiment, the crystalline form is grown at 4° C. In another embodiment, the ligand is cAMP. In one embodiment, the solution comprises 10 mM cAMP, 0.4 mM zinc sulfate, 15 mg/mL phosphodiesterase 4D2 (PDE4D2) in a storage buffer of 50 mM NaCl, 20 mM Tris-HCl (pH 7.5), and 1 mM β-mercaptoethanol. In still another embodiment, the method further comprises saturating cAMP binding by soaking the crystalline form in a buffer of 50 mM HEPES (pH 7.5), 20% PEG3350, 25% ethylene glycol, 0.4 mM zinc sulfate, and 50 mM cAMP. In one embodiment, the saturating occurs at room temperature.

The presently disclosed subject matter also provides a crystalline form formed by the methods of the presently disclosed subject matter.

The presently disclosed subject matter also provides a binding site in a human phosphodiesterase 4D2 (PDE4D2) catalytic domain polypeptide for a substrate, wherein the substrate is in van der Waals, hydrogen bonding, or both van der Waals and hydrogen bonding contact with at least one of the following residues of the human phosphodiesterase 4D2 (PDE4D2) polypeptide: Tyr159, His160, His164, His200, Asp201, Met273, Asp318, Leu319, Asn321, Thr333, Ile336, Phe340, Gln369, and Phe372. In one embodiment, the binding site comprises four phosphodiesterase 4D2 (PDE4D2) catalytic domain polypeptides. In another embodiment, at least two of the four phosphodiesterase 4D2 (PDE4D2) catalytic domain polypeptides are in van der Waals, hydrogen bonding, or both van der Waals and hydrogen bonding contact through at least one of the following residues: Arg116, Met147, Thr148, Asp151, Asn214, Thr215, Asn216, Glu218, Ala220, Leu221, Met222, Tyr223, Asn224, Asp225, Asn231, Leu234, Ala235, Lys239, Gln242, Glu243, Glu244, Lys254, Arg257, Gln258, Arg261, Ile265, Arg346, Glu349, and Arg350. In another embodiment, the binding site further comprises a metal ion.

The presently disclosed subject matter also provides a complex of a human phosphodiesterase 4D2 (PDE4D2) catalytic domain polypeptide and a substrate, wherein the substrate is in van der Waals, hydrogen bonding, or both van der Waals and hydrogen bonding contact with at least one of the following residues of the human phosphodiesterase 4D2 (PDE4D2) polypeptide: Tyr159, His160, His164, His200, Asp201, Met273, Asp318, Leu319, Asn321, Thr333, Ile336, Phe340, Gln369, and Phe372. In one embodiment, the complex comprises four phosphodiesterase 4D2 (PDE4D2) catalytic domain polypeptides. In another embodiment, at least two of the four phosphodiesterase 4D2 (PDE4D2) catalytic domain polypeptides are in van der Waals, hydrogen bonding, or both van der Waal and hydrogen bonding contact through one or more of the following residues: Arg116, Met147, Thr148, Asp151, Asn214, Thr215, Asn216, Glu218, Ala220, Leu221, Met222, Tyr223, Asn224, Asp225, Asn231, Leu234, Ala235, Lys239, Gln242, Glu243, Glu244, Lys254, Arg257, Gln258, Arg261, Ile265, Arg346, Glu349, and Arg350. In still another embodiment, the complex further comprises a metal ion.

The presently disclosed subject matter also provides a crystal of a complex of a human phosphodiesterase 4D2 (PDE4D2) catalytic domain polypeptide and a substrate. In one embodiment, the crystal has the following physical measurements: space group P2₁2₁2₁; and unit cell a=99.2 Å; b=111.2 Å; c=159.7 Å.

The presently disclosed subject matter also provides a method for identifying a phosphodiesterase ligand, the method comprising: (a) providing atomic coordinates of a phosphodiesterase 4D2 (PDE4D2) catalytic domain in complex with a ligand to a computerized modeling system; and (b) modeling a ligand that fits spatially into the binding site of the phosphodiesterase 4D2 (PDE4D2) catalytic domain to thereby identify a phosphodiesterase ligand. In one embodiment, the phosphodiesterase 4D2 (PDE4D2) catalytic domain comprises the amino acid sequence of SEQ ID NO: 4. In another embodiment, the method further comprises identifying in an assay for phosphodiesterase-mediated activity a modeled ligand that increases or decreases the activity of the phosphodiesterase.

The presently disclosed subject matter also provides a method of identifying a phosphodiesterase 4D2 (PDE4D2) ligand that selectively binds a phosphodiesterase 4D2 (PDE4D2) polypeptide compared to other polypeptides, the method comprising: (a) providing atomic coordinates of a phosphodiesterase 4D2 (PDE4D2) catalytic domain in complex with a ligand to a computerized modeling system; and (b) modeling a ligand that fits into the binding pocket of a phosphodiesterase 4D2 (PDE4D2) catalytic domain and that interacts with residues of a phosphodiesterase 4D2 (PDE4D2) catalytic domain that are conserved among phosphodiesterase 4D2 (PDE4D2) subtypes to thereby identify a phosphodiesterase 4D2 (PDE4D2) ligand that selectively binds a phosphodiesterase 4D2 (PDE4D2) polypeptide compared to other polypeptides. In one embodiment, the phosphodiesterase 4D2 (PDE4D2) catalytic domain comprises the amino acid sequence shown in SEQ ID NO: 4. In another embodiment, the method further comprises identifying in a biological assay for phosphodiesterase 4D2 (PDE4D2) activity a modeled ligand that selectively binds to said phosphodiesterase 4D2 (PDE4D2) and increases or decreases the activity of the phosphodiesterase 4D2 (PDE4D2).

The presently disclosed subject matter also provides a method for designing a ligand of a phosphodiesterase 4D2 (PDE4D2) polypeptide, the method comprising: (a) forming a complex of a compound bound to the phosphodiesterase 4D2 (PDE4D2) polypeptide; (b) determining a structural feature of the complex formed in (a); wherein the structural feature is of a binding site for the compound; and (c) using the structural feature determined in (b) to design a ligand of a phosphodiesterase 4D2 (PDE4D2) polypeptide capable of binding to the binding site of the presently disclosed subject matter. In one embodiment, the method further comprises using a computer-based model of the complex formed in (a) in designing the ligand.

The presently disclosed subject matter also provides a method of designing a ligand of a phosphodiesterase polypeptide, the method comprising: (a) selecting a candidate phosphodiesterase ligand; (b) determining which amino acid or amino acids of a phosphodiesterase polypeptide interact with the ligand using a three-dimensional model of a crystallized protein, the model comprising a phosphodiesterase 4D2 (PDE4D2) catalytic domain in complex with a ligand; (c) identifying in a biological assay for phosphodiesterase activity a degree to which the ligand modulates the activity of the phosphodiesterase polypeptide; (d) selecting a chemical modification of the ligand wherein the interaction between the amino acids of the phosphodiesterase polypeptide and the ligand is predicted to be modulated by the chemical modification; (e) synthesizing a ligand having the chemical modified to form a modified ligand; (f) contacting the modified ligand with the phosphodiesterase polypeptide; (g) identifying in a biological assay for phosphodiesterase activity a degree to which the modified ligand modulates the biological activity of the phosphodiesterase polypeptide; and (h) comparing the biological activity of the phosphodiesterase polypeptide in the presence of modified ligand with the biological activity of the phosphodiesterase polypeptide in the presence of the unmodified ligand, whereby a ligand of a phosphodiesterase polypeptide is designed. In one embodiment, the phosphodiesterase is phosphodiesterase 4D2 (PDE4D2). In another embodiment, the phosphodiesterase 4D2 (PDE4D2) polypeptide is a human phosphodiesterase 4D2 (PDE4D2) polypeptide. In another embodiment, the phosphodiesterase 4D2 (PDE4D2) polypeptide comprises the amino acid sequence of SEQ ID NO: 4. In another embodiment, the method further comprises repeating steps (a) through (f), if the biological activity of the phosphodiesterase polypeptide in the presence of the modified ligand varies from the biological activity of the phosphodiesterase polypeptide in the presence of the unmodified ligand.

The presently disclosed subject matter also provides a method of designing a chemical compound that modulates the biological activity of a target phosphodiesterase polypeptide, the method comprising: (a) obtaining three-dimensional structures for a catalytic domain (CD) of phosphodiesterase 4D2 (PDE4D2) bound to a ligand, wherein the structures are selected from the group consisting of X-ray structures and computer generated models; (b) rotating and translating the three-dimensional structures as rigid bodies so as to superimpose corresponding backbone atoms of a core region of the phosphodiesterase 4D2 (PDE4D2) CD; (c) comparing the superimposed three-dimensional structures to identify volume near a catalytic pocket of the PDE CD that is available to a ligand in one or more structures, but not available to the ligand in one or more other structures; (d) designing a chemical compound that could occupy the volume in some of the complexed structures, but not in others; (e) synthesizing the designed chemical compound; and (f) testing the designed chemical compound in a biological assay to determine whether it acts as a ligand of a phosphodiesterase with a desired effect on phosphodiesterase biological activities, whereby a ligand of a phosphodiesterase polypeptide is designed. In one embodiment, the method further comprises designing a chemical compound by considering a known ligand of the PDE CD and adding a substituent that protrudes into the volume identified in step (c) or that makes a desired interaction. In another embodiment, the phosphodiesterase is PDE4D2. In another embodiment, the designing a chemical compound further comprises using computer modeling software.

The presently disclosed subject matter also provides a method of designing a ligand that selectively modulates the activity of a phosphodiesterase polypeptide, the method comprising: (a) evaluating a three-dimensional structure of a crystallized phosphodiesterase 4D2 (PDE4D2) catalytic domain polypeptide in complex with a ligand; and (b) synthesizing a potential ligand based on the three-dimensional structure of the crystallized phosphodiesterase 4D2 (PDE4D2) catalytic polypeptide in complex with a ligand, whereby a ligand that selectively modulates the activity of a phosphodiesterase polypeptide is designed. In one embodiment, the phosphodiesterase is phosphodiesterase 4D2 (PDE4D2). In another embodiment, the phosphodiesterase 4D2 (PDE4D2) catalytic domain polypeptide comprises the amino acid sequence of SEQ ID NO: 4. In another embodiment, the crystallized phosphodiesterase 4D2 (PDE4D2) catalytic domain polypeptide is in an orthorhombic crystalline form. In another embodiment, the three-dimensional structure of the crystallized phosphodiesterase 4D2 (PDE4D2) catalytic domain polypeptide in complex with a ligand can be determined to a resolution of about 2.3 Å or better. In another embodiment, the method further comprises contacting a phosphodiesterase catalytic domain polypeptide with the potential ligand and a ligand; and assaying the phosphodiesterase catalytic domain polypeptide for binding of the potential ligand, for a change in activity of the phosphodiesterase catalytic domain polypeptide, or both.

The presently disclosed subject matter also provides a method of screening a plurality of compounds for a ligand of a phosphodiesterase 4D2 (PDE4D2) catalytic domain polypeptide, the method comprising: (a) providing a library of test samples; (b) contacting a crystalline form comprising a phosphodiesterase 4D2 (PDE4D2) polypeptide in complex with a ligand with each test sample; (c) detecting an interaction between a test sample and the crystalline phosphodiesterase 4D2 (PDE4D2) polypeptide in complex with a ligand; (d) identifying a test sample that interacts with the crystalline phosphodiesterase 4D2 (PDE4D2) polypeptide in complex with a ligand; and (e) isolating a test sample that interacts with the crystalline phosphodiesterase 4D2 (PDE4D2) polypeptide in complex with a ligand, whereby a plurality of compounds is screened for a ligand of a phosphodiesterase 4D2 (PDE4D2) catalytic domain polypeptide. In one embodiment, the phosphodiesterase 4D2 (PDE4D2) polypeptide comprises a phosphodiesterase 4D2 (PDE4D2) catalytic domain. In another embodiment, the phosphodiesterase 4D2 (PDE4D2) polypeptide is a human phosphodiesterase 4D2 (PDE4D2) polypeptide. In another embodiment, the phosphodiesterase 4D2 (PDE4D2) polypeptide comprises the amino acid sequence of SEQ ID NO: 4. In another embodiment, the library of test samples is bound to a substrate. In still another embodiment, the library of test samples is synthesized directly on a substrate.

The presently disclosed subject matter also provides a crystallized, recombinant polypeptide comprising: (a) an amino acid sequence set forth in SEQ ID NO: 2 or SEQ ID NO: 4; (b) an amino acid sequence having at least about 95% identity with the amino acid sequence set forth in SEQ ID NO: 2 or SEQ ID NO: 4; or (c) an amino acid sequence encoded by a polynucleotide that hybridizes under stringent conditions to the complementary strand of a polynucleotide having SEQ ID NO: 1 or SEQ ID NO: 3 and has at least one biological activity of PDE4D2; wherein the polypeptide of (a), (b) or (c) is in crystal form. In one embodiment, the complex is in crystal form. In another embodiment, the complex is in crystal form. In another embodiment, the crystallized, recombinant polypeptide diffracts x-rays to a resolution of about 3.5 Å or better. In another embodiment, the polypeptide comprises at least one heavy atom label. In another embodiment, the polypeptide is labeled with seleno-methionine.

The presently disclosed subject matter also provides a method for designing a modulator for the prevention or treatment of a disease or disorder, comprising: (a) providing a three-dimensional structure for a crystallized, recombinant polypeptide of claim 1; (b) identifying a potential modulator for the prevention or treatment of a disease or disorder by reference to the three-dimensional structure; (c) contacting a polypeptide of the composition of claim 1 or a phosphodiesterase (PDE) with the potential modulator; and (d) assaying the activity of the polypeptide after contact with the modulator, wherein a change in the activity of the polypeptide indicates that the modulator may be useful for prevention or treatment of a disease or disorder.

The presently disclosed subject matter also provides a method for obtaining structural information of a crystallized polypeptide, the method comprising: (a) crystallizing a recombinant polypeptide, wherein the polypeptide comprises: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; and wherein the crystallized polypeptide is capable of diffracting X-rays to a resolution of 3.5 Å or better; and (b) analyzing the crystallized polypeptide by X-ray diffraction to determine the three-dimensional structure of at least a portion of the crystallized polypeptide. In one embodiment, the three-dimensional structure of the portion of the crystallized polypeptide is determined to a resolution of 3.5 Å or better.

The presently disclosed subject matter also provides a method for identifying a druggable region of a polypeptide, the method comprising: (a) obtaining crystals of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2, such that the three dimensional structure of the crystallized polypeptide may be determined to a resolution of 3.5 Å or better; (b) determining the three dimensional structure of the crystallized polypeptide using X-ray diffraction; and (c) identifying a druggable region of the crystallized polypeptide based on the three-dimensional structure of the crystallized polypeptide. In one embodiment, the druggable region is an active site. In another embodiment, the druggable region is on the surface of the polypeptide.

The presently disclosed subject matter also provides a crystallized polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; wherein the crystal has a P2₁2₁2₁ space group.

The presently disclosed subject matter also provides a crystallized polypeptide comprising a structure of a polypeptide that is defined by a substantial portion of the atomic coordinates set forth in Table 4 or Table 5.

The presently disclosed subject matter also provides a method for determining the crystal structure of a homolog of a polypeptide, the method comprising: (a) providing the three dimensional structure of a first crystallized polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; (b) obtaining crystals of a second polypeptide comprising an amino acid sequence that is at least 70% identical to the amino acid sequence set forth in SEQ ID NO: 2 or SEQ ID NO: 4, such that the three dimensional structure of the second crystallized polypeptide may be determined to a resolution of 3.5 Å or better; and (c) determining the three dimensional structure of the second crystallized polypeptide by x-ray crystallography based on the atomic coordinates of the three dimensional structure provided in step (a). In one embodiment, the atomic coordinates for the second crystallized polypeptide have a root mean square deviation from the backbone atoms of the first polypeptide of not more than 1.5 Å for all backbone atoms shared in common with the first polypeptide and the second polypeptide.

The presently disclosed subject matter also provides a method for homology modeling a homolog of human PDE4D2, comprising: (a) aligning the amino acid sequence of a homolog of human PDE4D2 with an amino acid sequence of SEQ ID NO: 2 or SEQ ID NO: 4 and incorporating the sequence of the homolog of human PDE4D2 into a model of human PDE4D2 derived from structure coordinates as listed in Table 4 or Table 5 to yield a preliminary model of the homolog of human PDE4D2; (b) subjecting the preliminary model to energy minimization to yield an energy minimized model; (c) remodeling regions of the energy minimized model where stereochemistry restraints are violated to yield a final model of the homolog of human PDE4D2.

The presently disclosed subject matter also provides a method for obtaining structural information about a molecule or a molecular complex of unknown structure comprising: (a) crystallizing the molecule or molecular complex; (b) generating an x-ray diffraction pattern from the crystallized molecule or molecular complex; and (c) applying at least a portion of the structure coordinates set forth in Table 4 or Table 5 to the x-ray diffraction pattern to generate a three-dimensional electron density map of at least a portion of the molecule or molecular complex whose structure is unknown.

The presently disclosed subject matter also provides a method for attempting to make a crystallized complex comprising a polypeptide and a modulator having a molecular weight of less than 5 kDa, the method comprising: (a) crystallizing a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; such that crystals of the crystallized polypeptide will diffract x-rays to a resolution of 5 Å or better; and (b) soaking the crystals in a solution comprising a potential modulator having a molecular weight of less than 5 kDa.

The presently disclosed subject matter also provides a method for incorporating a potential modulator in a crystal of a polypeptide, comprising placing a hexagonal crystal of human PDE4D2 having unit cell dimensions a=99.2 Å; b=111.2 Å; c=159.7 Å, α=β=γ=90°, with an orthorhombic space group P2₁2₁2₁, in a solution comprising the potential modulator.

The presently disclosed subject matter also provides a computer readable storage medium comprising digitally encoded structural data, wherein the data comprises structural coordinates as listed in Table 4 or Table 5 for the backbone atoms of at least about six amino acid residues from a druggable region of human PDE4D2.

The presently disclosed subject matter also provides a scalable three-dimensional configuration of points, at least a portion of the points derived from some or all of the structure coordinates as listed in Table 4 or Table 5 for a plurality of amino acid residues from a druggable region of human PDE4D2. In one embodiment, the structure coordinates as listed in Table 4 or Table 5 for the backbone atoms of at least about five amino acid residues from a druggable region of human PDE4D2 are used to derive part or all of the portion of points. In another embodiment, the structure coordinates as listed in Table 4 or Table 5 for the backbone and optionally the side chain atoms of at least about ten amino acid residues from a druggable region of human PDE4D2 are used to derive part or all of the portion of points. In another embodiment, the structure coordinates as listed in Table 4 or Table 5 for the backbone atoms of at least about fifteen amino acid residues from a druggable region of human PDE4D2 are used to derive part or all of the portion of points. In another embodiment, substantially all of the points are derived from structure coordinates as listed in Table 4 or Table 5. In still another embodiment, the structure coordinates as listed in Table 4 or Table 5 for the atoms of the amino acid residues from any of the above-described druggable regions of human PDE4D2 are used to derive part or all of the portion of points.

The presently disclosed subject matter also provides a scalable three-dimensional configuration of points, comprising points having a root mean square deviation of less than about 1.5 Å from the three dimensional coordinates as listed in Table 4 or Table 5 for the backbone atoms of at least five amino acid residues, wherein the five amino acid residues are from a druggable region of human PDE4D2. In one embodiment, any point-to-point distance, calculated from the three dimensional coordinates as listed in Table 4 or Table 5, between one of the backbone atoms for one of the five amino acid residues and another backbone atom of a different one of the five amino acid residues is not more than about 10 Å.

The presently disclosed subject matter also provides a scalable three-dimensional configuration of points comprising points having a root mean square deviation of less than about 1.5 Å from the three dimensional coordinates as listed in Table 4 or Table 5 for the atoms of the amino acid residues from any of the above-described druggable regions of human PDE4D2.

The presently disclosed subject matter also provides a computer readable storage medium comprising digitally encoded structural data, wherein the data comprise the identity and three-dimensional coordinates as listed in Table 4 or Table 5 for the atoms of the amino acid residues from any of the above-described druggable regions of human PDE4D2.

The presently disclosed subject matter also provides a scalable three-dimensional configuration of points, wherein the points have a root mean square deviation of less than about 1.5 Å from the three dimensional coordinates as listed in Table 4 or Table 5 for the atoms of the amino acid residues from any of the above-described druggable regions of human PDE4D2, wherein up to one amino acid residue in each of the regions may have a conservative substitution thereof.

The presently disclosed subject matter also provides a scalable three-dimensional configuration of points derived from a druggable region of a polypeptide, wherein the points have a root mean square deviation of less than about 1.5 Å from the three dimensional coordinates as listed in Table 4 or Table 5 for the backbone atoms of at least ten amino acid residues that participate in the intersubunit contacts of human PDE4D2.

The presently disclosed subject matter also provides a computer-assisted method for identifying an inhibitor of the activity of human PDE4D2, comprising: (a) supplying a computer modeling application with a set of structure coordinates as listed in Table 4 or Table 5 for the atoms of the amino acid residues from any of the above-described druggable regions of human PDE4D2 so as to define part or all of a molecule or complex; (b) supplying the computer modeling application with a set of structure coordinates of a chemical entity; and (c) determining whether the chemical entity is expected to bind to or interfere with the molecule or complex. In one embodiment, determining whether the chemical entity is expected to bind to or interfere with the molecule or complex comprises performing a fitting operation between the chemical entity and a druggable region of the molecule or complex, followed by computationally analyzing the results of the fitting operation to quantify the association between the chemical entity and the druggable region. In another embodiment, the method further comprises screening a library of chemical entities.

The presently disclosed subject matter also provides a computer-assisted method for designing an inhibitor of PDE4D2 activity comprising: (a) supplying a computer modeling application with a set of structure coordinates having a root mean square deviation of less than about 1.5 Å from the structure coordinates as listed in Table 4 or Table 5 for the atoms of the amino acid residues from any of the above-described druggable regions of human PDE4D2 so as to define part or all of a molecule or complex; (b) supplying the computer modeling application with a set of structure coordinates for a chemical entity; (c) evaluating the potential binding interactions between the chemical entity and the molecule or complex; (d) structurally modifying the chemical entity to yield a set of structure coordinates for a modified chemical entity; and (e) determining whether the modified chemical entity is an inhibitor expected to bind to or interfere with the molecule or complex, wherein binding to or interfering with the molecule or molecular complex is indicative of potential inhibition of PDE4D2 activity. In one embodiment, determining whether the modified chemical entity is an inhibitor expected to bind to or interfere with the molecule or complex comprises performing a fitting operation between the chemical entity and the molecule or complex, followed by computationally analyzing the results of the fitting operation to evaluate the association between the chemical entity and the molecule or complex. In another embodiment, the set of structure coordinates for the chemical entity is obtained from a chemical library.

The presently disclosed subject matter also provides a computer-assisted method for designing an inhibitor of PDE4D2 activity de novo comprising: (a) supplying a computer modeling application with a set of three-dimensional coordinates derived from the structure coordinates as listed in Table 4 or Table 5 for the atoms of the amino acid residues from any of the above-described druggable regions of human PDE4D2 so as to define part or all of a molecule or complex; (b) computationally building a chemical entity represented by a set of structure coordinates; and (c) determining whether the chemical entity is an inhibitor expected to bind to or interfere with the molecule or complex, wherein binding to or interfering with the molecule or complex is indicative of potential inhibition of PDE4D2 activity. In one embodiment, determining whether the chemical entity is an inhibitor expected to bind to or interfere with the molecule or complex comprises performing a fitting operation between the chemical entity and a druggable region of the molecule or complex, followed by computationally analyzing the results of the fitting operation to quantify the association between the chemical entity and the druggable region. In one embodiment, the method further comprises supplying or synthesizing the potential inhibitor, then assaying the potential inhibitor to determine whether it inhibits PDE4D2 activity.

The presently disclosed subject matter also provides a method for identifying a potential modulator for the prevention or treatment of a disease or disorder, the method comprising: (a) providing the three dimensional structure of a crystallized polypeptide comprising: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; (b) obtaining a potential modulator for the prevention or treatment of a disease or disorder based on the three dimensional structure of the crystallized polypeptide; (c) contacting the potential modulator with a second polypeptide comprising: (i) an amino acid sequence set forth in SEQ ID NO: 2 or SEQ ID NO: 4; (ii) an amino acid sequence having at least about 95% identity with the amino acid sequence set forth in SEQ ID NO: 2 or SEQ ID NO: 4; or (iii) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; which second polypeptide may optionally be the same as the crystallized polypeptide; and (d) assaying the activity of the second polypeptide, wherein a change in the activity of the second polypeptide indicates that the compound may be useful for prevention or treatment of a disease or disorder.

The presently disclosed subject matter also provides a method for designing a candidate modulator for screening for inhibitors of a polypeptide, the method comprising: (a) providing the three dimensional structure of a druggable region of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; and (b) designing a candidate modulator based on the three dimensional structure of the druggable region of the polypeptide.

The presently disclosed subject matter also provides a method for identifying a potential modulator of a polypeptide from a database, the method comprising: (a) providing the three-dimensional coordinates for a plurality of the amino acids of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; (b) identifying a druggable region of the polypeptide; and (c) selecting from a database at least one potential modulator comprising three dimensional coordinates which indicate that the modulator may bind or interfere with the druggable region. In one embodiment, the modulator is a small molecule.

The presently disclosed subject matter also provides a method for preparing a potential modulator of a druggable region contained in a polypeptide, the method comprising: (a) using the atomic coordinates for the backbone atoms of at least about six amino acid residues from a polypeptide of SEQ ID NO: 4, with a±a root mean square deviation from the backbone atoms of the amino acid residues of not more than 1.5 Å, to generate one or more three-dimensional structures of a molecule comprising a druggable region from the polypeptide; (b) employing one or more of the three dimensional structures of the molecule to design or select a potential modulator of the druggable region; and (c) synthesizing or obtaining the modulator.

The presently disclosed subject matter also provides an apparatus for determining whether a compound is a potential modulator of a polypeptide, the apparatus comprising: (a) a memory that comprises: (i) the three dimensional coordinates and identities of at least about fifteen atoms from a druggable region of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; (ii) executable instructions; and (b) a processor that is capable of executing instructions to: (i) receive three-dimensional structural information for a candidate modulator; (ii) determine if the three-dimensional structure of the candidate modulator is complementary to the three dimensional coordinates of the atoms from the druggable region; and (iii) output the results of the determination.

The presently disclosed subject matter also provides a method for making an inhibitor of PDE4D2 activity, the method comprising chemically or enzymatically synthesizing a chemical entity to yield an inhibitor of PDE4D2 activity, the chemical entity having been identified during a computer-assisted process comprising supplying a computer modeling application with a set of structure coordinates of a molecule or complex, the molecule or complex comprising at least a portion of at least one druggable region from human PDE4D2; supplying the computer modeling application with a set of structure coordinates of a chemical entity; and determining whether the chemical entity is expected to bind or to interfere with the molecule or complex at a druggable region, wherein binding to or interfering with the molecule or complex is indicative of potential inhibition of PDE4D2 activity.

The presently disclosed subject matter also provides a computer readable storage medium comprising digitally encoded data, wherein the data comprises structural coordinates for a druggable region that is structurally homologous to the structure coordinates as listed in Table 4 or Table 5 for a druggable region of human PDE4D2.

The presently disclosed subject matter also provides a computer readable storage medium comprising digitally encoded structural data, wherein the data comprise a majority of the three-dimensional structure coordinates as listed in Table 4 or Table 5. In one embodiment, the computer readable storage medium further comprises the identity of the atoms for the majority of the three-dimensional structure coordinates as listed in Table 4 or Table 5. In another embodiment, the data comprise substantially all of the three-dimensional structure coordinates as listed in Table 4 or Table 5.

Accordingly, it is an object of the presently disclosed subject matter to provide three-dimensional structures of an unliganded PDE4D2 catalytic domain and of a PDE4D2 catalytic domain in complex with a ligand. The object is achieved in whole or in part by the presently disclosed subject matter.

An object of the presently disclosed subject matter having been stated hereinabove, other objects will be evident as the description proceeds, when taken in connection with the accompanying Drawings and Examples as described hereinbelow.

### BRIEF DESCRIPTION OF THE DRAWINGS

FIGS. 1A-1C depict the catalytic domain of PDE4D2.

FIG. 1A is a ribbon diagram of monomeric PDE4D2. AMP is shown in stick form while two divalent metals are indicated by spheres.

FIG. 1B depicts a tetramer of PDE4D2. In each monomer, AMP is depicted as small spheres and metal ions are depicted as large spheres.

FIG. 1C is a comparison of the sequences of the catalytic domain of two PDE4 molecules. The metal binding residues (His164, His200, Asp201, and Asp318) are in bold while the AMP binding residues are underlined. The bars above the sequences represent helices common to both PDE4B and PDE4D. The most C-terminal bar indicates a helix present only in PDE4B.

FIGS. 2A and 2B depict AMP binding.

FIG. 2A is a stereoview of electron density for AMP, which was calculated from the omitted (Fo−Fc) map and contoured at 3.5 sigmas.

FIG. 2B depicts AMP interactions with the active site residues. The metal binding residues are shown in purple.

FIG. 3 depicts the interactions of the metal ions of PDE4 with AMP. Dotted lines represent the hydrogen bonds to the metals. The hydrogen bonds between the phosphate of AMP and water molecules W3, W4, and W5 are not shown. Me2 represents the location of the second metal ion.

FIG. 4 illustrates a putative mechanism for the hydrolysis of the phosphodiester bond by PDE4.

### BRIEF DESCRIPTION OF THE SEQUENCES IN THE SEQUENCE LISTING

SEQ ID NO:1 is a nucleotide sequence encoding a human PDE4D2 polypeptide (GenBank accession number AF012074).

SEQ ID NO:2 is the amino acid sequence encoded by SEQ ID NO:1.

SEQ ID NO:3 is a nucleotide sequence encoding a PDE4D2 catalytic domain polypeptide, the polypeptide corresponding to amino acids 79-438 of the human PDE4D2 polypeptide.

SEQ ID NO: 4 is the amino acid sequence encoded by SEQ ID NO:3.

### DETAILED DESCRIPTION

Cyclic nucleotide phosphodiesterases (PDEs) regulate the intracellular concentrations of cyclic 3′,5′-adenosine and guanosine monophosphate (cAMP and cGMP, respectively) by hydrolyzing them to AMP and GMP. Family-selective inhibitors of PDEs have been studied for treatment of various human diseases. However, the catalytic mechanism of cyclic nucleotide hydrolysis by PDE is not clear. Disclosed herein in alternative embodiments are the resolutions of two crystal structures of a human PDE4D2 catalytic domain at 2.3 Å resolution: one unliganded and one in complex with AMP. In the representative structure of PDE4D2-AMP, two divalent metal ions simultaneously interact with the phosphate group of AMP, implying a binuclear catalysis. In addition, the structure revealed a water molecule that binds to the second metal ion and forms hydrogen bonds with Glu230 and a phosphate oxygen of AMP. While the co-inventors do not wish to be bound by any particular theory of operation, a catalytic mechanism in which Glu230, a conserved residue in all PDEs, activates this water molecule to serve as a nucleophile for the hydrolysis of the cAMP phosphodiester bond is proposed.

Until disclosure of the presently disclosed subject matter presented herein, the ability to obtain crystalline forms of a PDE4D2 catalytic domain, particularly in a complex with a substrate/product, has not been realized. And until the present disclosure, a detailed three-dimensional crystal structure of an unbound PDE4D2 catalytic domain polypeptide and a PDE4D2 catalytic domain polypeptide in complex with a substrate/product has not been solved.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and degradation associated with solution storage. Crystalline polypeptides can also be useful as pharmaceutical preparations. Finally, crystallization techniques are generally free of problems such as denaturation associated with other stabilization methods (i.e., lyophilization).

Once crystallization has been accomplished, crystallographic data provides useful structural information that can assist the design of compounds that can serve as agonists or antagonists, as described herein below. In addition, the crystal structure provides information that can be used to map the molecular surface of the catalytic domain of PDE4D2. A small non-peptide molecule designed to mimic portions of this surface could serve as a modulator of PDE4D2 catalytic activity.

I. Definitions

Before the present proteins, nucleotide sequences, and methods are described, it is understood that the presently disclosed subject matter is not limited to the particular methodology, protocols, cell lines, vectors, and reagents described as these can vary. It is also to be understood that the terminology used herein is for the purpose of describing particular embodiments only, and is not intended to limit the scope of the presently disclosed subject matter.

Unless defined otherwise, all technical and scientific terms used herein are intended to have their ordinary meanings as understood by one of ordinary skill in the art to which presently disclosed subject matter pertains. Although any methods and materials similar or equivalent to those described herein can be used in the practice or testing of the presently disclosed subject matter, the preferred methods, devices, and materials are now described. All publications mentioned herein are incorporated by reference for the purpose of describing the cell lines, vectors, reagents, and methodologies they disclose.

For convenience, certain terms employed in the specification, examples, and appended claims are collected here. Unless defined otherwise, all technical and scientific terms used herein have the same meaning as commonly understood by one of ordinary skill in the art to which presently disclosed subject matter belongs.

Following long-standing patent law convention, the articles “a” and “an” are used herein to refer to one or to more than one (i.e., to at least one) of the grammatical object of the article. By way of example, “an element” means one element or more than one element.

As used herein, the term “agonist” refers to an agent that supplements or potentiates a biological activity of a functional PDE4D2 gene or protein, or of a polypeptide encoded by a gene that is up- or down-regulated by a PDE4D2 polypeptide, and/or a polypeptide encoded by a gene that contains a PDE4D2 binding site or response element in its promoter region.

As used herein, the term “antagonist” refers to an agent that decreases or inhibits the biological activity of a functional gene or protein (for example, a functional PDE4D2 gene or protein), or that supplements or potentiates the biological activity of a naturally occurring or engineered non-functional gene or protein (for example, a non-functional PDE4D2 gene or protein). Alternatively, an antagonist can decrease or inhibit the biological activity of a functional gene or polypeptide encoded by a gene that is up or down regulated by a PDE4D2 polypeptide. An antagonist can also supplement or potentiate the biological activity of a naturally occurring or engineered non-functional gene or polypeptide encoded by a gene that is up or down regulated by a PDE4D2 polypeptide.

As used herein, the terms “α-helix” and “alpha-helix” are used interchangeably and refer to a conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turn of the helix, which extends about 0.56 nm along the long axis.

As used herein, the terms “amino acid”, “amino acid residue”, and “residue” are used interchangeably and refer to an amino acid formed upon chemical digestion (hydrolysis) of a peptide or polypeptide at its peptide linkages. Amino acids can also be synthesized individually or as components of a peptide. In one embodiment, the amino acid residues described herein are in the “L” isomeric form. However, residues in the “D” isomeric form can be substituted for any L-amino acid residue, provided that the desired functional property is retained by the polypeptide. In the context of an amino acid, NH₂ refers to the free amino group present at the amino terminus of a polypeptide, although some amino acids can have NH₂ groups at other positions in the amino acid. COOH refers to the free carboxy group present at the carboxy terminus of a polypeptide. In keeping with standard polypeptide nomenclature, abbreviations for amino acid residues are presented above. The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of the foregoing.

It is noted that amino acid residue sequences represented herein by formulae have a left-to-right orientation in the conventional direction of amino terminus to carboxy terminus. In addition, the terms “amino acid”, “amino acid residue”, and “residue” are broadly defined to include the amino acids listed in the above table and modified or unusual amino acids. Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the terms “β-sheet” and “beta-sheet” are used interchangeably and refer to the conformation of a polypeptide chain stretched into an extended zigzag conformation. Portions of polypeptide chains that run “parallel” all run in the same direction. Polypeptide chains that are “anti-parallel” run in the opposite direction from the parallel chains or from each other.

The term “binding” refers to an association, which may be a stable association, between two molecules, i.e., between a polypeptide of the presently disclosed subject matter and a binding partner, due to, for example, electrostatic, hydrophobic, ionic, and/or hydrogen-bond interactions under physiological conditions.

As used herein, the terms “binding site of the PDE4D2 catalytic domain”, “PDE4D2 catalytic site”, and “PDE4D2 binding site” are used interchangeably, and refer to a cavity within the PDE4D2 catalytic domain where a ligand (i.e. cAMP) binds. This cavity can be empty, or can contain water molecules or other molecules from the solvent, or can contain ligand atoms. The “main” binding pocket includes the region of space not occupied by atoms of PDE4D2 that is approximately encompassed or bounded by PDE4D2 residues Tyr159, His160, His164, His200, Asp201, Met273, Asp318, Leu319, Asn321, Thr333, Ile336, Phe340, Gln369, and Phe372. The binding pocket also includes small regions near to and contiguous with the “main” binding pocket that not occupied by atoms of PDE4D2.

As used herein the term “biological activity” refers to any biochemical function of a biological molecule. A biological activity includes, but is not limited to an interaction with another biological molecule (for example, a polypeptide, a nucleic acid, or a combination thereof). As such, a biological activity results in a biochemical effect including, but not limited to the hydrolysis of a cyclic nucleoside monophosphate.

A “comparison window,” as used herein, refers to a conceptual segment of at least 20 contiguous amino acid positions wherein a protein sequence may be compared to a reference sequence of at least 20 contiguous amino acids and wherein the portion of the protein sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package, available from Accelrys, Inc., San Diego, Calif., United States of America), or by inspection, and the best alignment (i.e., resulting in the highest percentage of homology over the comparison window) generated by the various methods may be identified.

The term “complex” refers to an association between at least two moieties (i.e. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as an antigen or ligand. “Protein complex” or “polypeptide complex” refers to a complex comprising at least one polypeptide.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gin and Pro, (ix) an aliphatic group consisting of Val, Leu, Ile, Met and Cys, and (x) a small hydroxyl group consisting of Ser and Thr.

As used herein, the term “DNA segment” refers to a DNA molecule that has been isolated free of total genomic DNA of a particular species. In one embodiment, a DNA segment encoding a PDE4D2 polypeptide refers to a nucleic acid comprising SEQ ID NO: 1. In another embodiment, a DNA segment encoding a PDE4D2 polypeptide refers to a nucleic acid comprising SEQ ID NO: 3. DNA segments can comprise a portion of a recombinant vector, including, for example, a plasmid, a cosmid, a phage, a virus, and the like.

As used herein, the term “DNA sequence encoding a PDE4D2 polypeptide” also refers to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions, or deletions, all of which still code for polypeptides having substantially the same activity.

The term “domain”, when used in connection with a polypeptide, refers to a specific region within such polypeptide that comprises a particular structure or mediates a particular function. In the typical case, a domain of a polypeptide of the presently disclosed subject matter is a fragment of the polypeptide. In certain instances, a domain is a structurally stable domain, as evidenced, for example, by mass spectroscopy, or by the fact that a modulator may bind to a druggable region of the domain.

The term “druggable region”, when used in reference to a polypeptide, nucleic acid, complex and the like, refers to a region of the molecule which is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which may be naturally occurring. In other instances, the druggable region is on the surface of the molecule.

Druggable regions may be described and characterized in a number of ways. For example, a druggable region may be characterized by some or all of the amino acids that make up the region, or the backbone atoms thereof, or the side chain atoms thereof (optionally with or without the Cα atoms). Alternatively, in certain instances, the volume of a druggable region corresponds to that of a carbon based molecule of at least about 200 amu and often up to about 800 amu. In other instances, it will be appreciated that the volume of such region may correspond to a molecule of at least about 600 amu and often up to about 1600 amu or more.

Alternatively, a druggable region may be characterized by comparison to other regions on the same or other molecules. For example, the term “affinity region” refers to a druggable region on a molecule (such as a polypeptide of the presently disclosed subject matter) that is present in several other molecules, in so much as the structures of the same affinity regions are sufficiently the same so that they are expected to bind the same or related structural analogs. An example of an affinity region is an ATP-binding site of a protein kinase that is found in several protein kinases (whether or not of the same origin). The term “selectivity region” refers to a druggable region of a molecule that may not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator may bind to the same affinity region across a number of proteins that have a substantially similar biological function, whereas the same modulator may bind to only one selectivity region of one of those proteins.

Continuing with examples of different druggable regions, the term “undesired region” refers to a druggable region of a molecule that upon interacting with another molecule results in an undesirable affect. For example, a binding site that oxidizes the interacting molecule (such as cytochrome P450 activity) and thereby results in increased toxicity for the oxidized molecule may be deemed a “undesired region”. Other examples of potential undesired regions includes regions that upon interaction with a drug decrease the membrane permeability of the drug, increase the excretion of the drug, or increase the blood brain transport of the drug. It may be the case that, in certain circumstances, an undesired region will no longer be deemed an undesired region because the affect of the region will be favorable, i.e., a drug intended to treat a brain condition would benefit from interacting with a region that resulted in increased blood brain transport, whereas the same region could be deemed undesirable for drugs that were not intended to be delivered to the brain.

When used in reference to a druggable region, the “selectivity” or “specificity’ of a molecule such as a modulator to a druggable region may be used to describe the binding between the molecule and a druggable region. For example, the selectivity of a modulator with respect to a druggable region may be expressed by comparison to another modulator, using the respective values of K_{d} (i.e., the dissociation constants for each modulator-druggable region complex) or, in cases where a biological effect is observed below the K_{d}, the ratio of the respective EC₅₀'s (i.e., the concentrations that produce 50% of the maximum response for the modulator interacting with each druggable region).

As used herein, the term “expression” generally refers to the cellular processes by which a biologically active polypeptide is produced. As such, the term “expression” generally includes those cellular processes that begin with transcription and end with the production of a functional polypeptide. As used herein, “expression” is also intended to refer to cellular processes by which a polypeptide is produced that would otherwise be functional except for the presence of mutations in the nucleotide sequence encoding it. Consistent with this usage, “expression” includes, but is not limited to such processes as transcription, translation, post-translational modification, and transport of a polypeptide.

A “fusion protein” or “fusion polypeptide” refers to a chimeric protein as that term is known in the art and may be constructed using methods known in the art. In many examples of fusion proteins, there are two different polypeptide sequences, and in certain cases, there may be more. The sequences may be linked in frame. A fusion protein may include a domain that is found (albeit in a different protein) in an organism that also expresses the first protein, or it may be an “interspecies”, “intergenic”, etc. fusion expressed by different kinds of organisms. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. The fusion polypeptides may be fused to the N-terminus, the C-terminus, or the N- and C-terminus of the first polypeptide. Exemplary fusion proteins include polypeptides comprising a glutathione S-transferase tag (GST-tag), histidine tag (His-tag), an immunoglobulin domain or an immunoglobulin binding domain.

As used herein, the term “gene” is used for simplicity to refer to nucleotide sequence that encodes a protein, polypeptide, or peptide. As such, the term “gene” refers to a nucleic acid comprising an open reading frame encoding a polypeptide having exon sequences and optionally intron sequences. The term “intron” refers to a DNA sequence present in a given gene that is not translated into protein and is generally found between exons. As will be understood by those of skill in the art, this functional term includes both genomic sequences and cDNA sequences. Representative embodiments of such sequences are disclosed herein.

The term “having substantially similar biological activity”, when used in reference to two polypeptides, refers to a biological activity of a first polypeptide which is substantially similar to at least one of the biological activities of a second polypeptide. A substantially similar biological activity means that the polypeptides carry out a similar function, i.e., a similar enzymatic reaction or a similar physiological process, etc. For example, two homologous proteins may have a substantially similar biological activity if they are involved in a similar enzymatic reaction, i.e., they are both kinases which catalyze phosphorylation of a substrate polypeptide, however, they may phosphorylate different regions on the same protein substrate or different substrate proteins altogether. Alternatively, two homologous proteins may also have a substantially similar biological activity if they are both involved in a similar physiological process, i.e., transcription. For example, two proteins may be transcription factors, however, they may bind to different DNA sequences or bind to different polypeptide interactors. Substantially similar biological activities may also be associated with proteins carrying out a similar structural role, for example, two membrane proteins.

As used herein, the term “interact” refers to detectable interactions between molecules, such as can be detected using, for example, a yeast two-hybrid assay. The term “interact” is also meant to include “binding” interactions between molecules. Interactions include, but are not limited to protein-protein, protein-nucleic acid, and protein-small molecule interactions. These interactions can be in the form of covalent or non-covalent interactions including, but not limited to ionic, hydrogen bonding, and van der Waals interactions.

As used herein, the term “isolated” refers to a nucleic acid substantially free of other nucleic acids, proteins, lipids, carbohydrates, or other materials with which it can be associated, such association being either in cellular material or in a synthesis medium. The term can also be applied to polypeptides, in which case the polypeptide is substantially free of nucleic acids, carbohydrates, lipids, and other undesired polypeptides. The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment, optionally covalently or non-covalently, of a detectable marker into a molecule, such as a polypeptide. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (i.e., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

The term “mammal” is known in the art, and exemplary mammals include humans, primates, bovines, porcines, canines, felines, and rodents (i.e., mice and rats).

The term “modulation”, when used in reference to a functional property or biological activity or process (i.e., enzyme activity or receptor binding), refers to the capacity to either up regulate (i.e., activate or stimulate), down regulate (i.e., inhibit or suppress) or otherwise change a quality of such property, activity, or process. In certain instances, such regulation may be contingent on the occurrence of a specific event, such as activation of a signal transduction pathway, and/or may be manifest only in particular cell types.

The term “modulator” refers to a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (i.e., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

As used herein, the term “molecular replacement” refers to a method that involves generating a preliminary model of the wild-type PDE4D2 catalytic domain or a PDE4D2 mutant crystal the structure for which coordinates are unknown, by orienting and positioning a molecule the structure for which coordinates are known within the unit cell of the unknown crystal so as best to account for the observed diffraction pattern of the unknown crystal. Phases can then be calculated from this model and combined with the observed amplitudes to give an approximate Fourier synthesis of the structure the coordinates for which are unknown. This, in turn, can be subjected to any of the several forms of refinement known in the art to provide a final, accurate structure of the unknown crystal. (Lattman, Meth Enzymol, 115:55-77, 1985; Rossmann, ed., The Molecular Replacement Method, Gordon & Breach, New York, 1972.) Using the structure coordinates of the catalytic domain of PDE4D2 provided by presently disclosed subject matter, molecular replacement can be used to determine the structure coordinates of a crystal of a mutant or of a homologue of the PDE4D2 catalytic domain, or of a different crystal form of the PDE4D2 catalytic domain.

The term “motif” refers to an amino acid sequence that is commonly found in a protein of a particular structure or function. Typically, a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases may be searched with a consensus sequence in order to identify other proteins containing a particular motif. Various search algorithms and/or programs may be used, including FASTA, BLAST or ENTREZ. FASTA and BLAST are available as a part of the GCG sequence analysis package (Accelrys, Inc., San Diego, Calif., United States of America). ENTREZ is available through the National Center for Biotechnology Information, National Library of Medicine, National Institutes of Health, Bethesda, Md., United States of America.

As used herein, the term “mutation” carries its traditional connotation and refers to a change, inherited, naturally occurring, or introduced, in a nucleic acid or polypeptide sequence, and is used in its sense as generally known to those of skill in the art.

The term “naturally occurring”, as applied to an object, refers to the fact that an object may be found in nature. For example, a polypeptide or polynucleotide sequence that is present in an organism (including bacteria) that may be isolated from a source in nature and which has not been intentionally modified by man in the laboratory is naturally occurring.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “nucleic acid of the presently disclosed subject matter” refers to a nucleic acid encoding a polypeptide of the presently disclosed subject matter, i.e., a nucleic acid comprising a sequence consisting of, or consisting essentially of, the polynucleotide sequence set forth in SEQ ID NO: 1 or SEQ ID NO: 3. A nucleic acid of the presently disclosed subject matter may comprise all, or a portion of: the nucleotide sequence of SEQ ID NO: 1 or SEQ ID NO: 3; a nucleotide sequence at least 60%, 70%, 80%, 90%, 95%, 96%, 97%, 98% or 99% identical to SEQ ID NO: 1 or SEQ ID NO: 3; a nucleotide sequence that hybridizes under stringent conditions to SEQ ID NO: 1 or SEQ ID NO: 3; nucleotide sequences encoding polypeptides that are functionally equivalent to polypeptides of the presently disclosed subject matter; nucleotide sequences encoding polypeptides at least about 60%, 70%, 80%, 85%, 90%, 95%, 98%, 99% homologous or identical with an amino acid sequence of SEQ ID NO: 2 or SEQ ID NO: 4; nucleotide sequences encoding polypeptides having an activity of a polypeptide of the presently disclosed subject matter and having at least about 60%, 70%, 80%, 85%, 90%, 95%, 98%, 99% or more homology or identity with SEQ ID NO: 2 or SEQ ID NO: 4; nucleotide sequences that differ by 1 to about 2, 3, 5, 7, 10, 15, 20, 30, 50, 75 or more nucleotide substitutions, additions or deletions, such as allelic variants, of SEQ ID NO: 1 and SEQ ID NO: 3; nucleic acids derived from and evolutionarily related to SEQ ID NO: 1 or SEQ ID NO: 3; and complements of, and nucleotide sequences resulting from the degeneracy of the genetic code, for all of the foregoing and other nucleic acids of the presently disclosed subject matter. Nucleic acids of the presently disclosed subject matter also include homologs, i.e., orthologs and paralogs, of SEQ ID NO: 1 or SEQ ID NO: 3 and also variants of SEQ ID NO: 1 or SEQ ID NO: 3 which have been codon optimized for expression in a particular organism (i.e., host cell).

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (i.e., inducers and polymerases) are bound to the control or regulatory sequence(s).

As used herein, “orthorhombic unit cell” refers to a unit cell wherein a≠b≠c; and α=β=γ=90°. The vectors a, b, and c describe the unit cell edges and the angles α, β, and γ describe the unit cell angles.

As used herein, the term “PDE4D2” refers to any polypeptide with an amino acid sequence that can be aligned with at least one of human, mouse, or rat PDE4D2, such that at least 50% of the amino acids are identical to the corresponding amino acid in the human, mouse, or rat PDE4D2. The term “PDE4D2” also encompasses nucleic acids for which the corresponding translated protein sequence can be considered to be a PDE4D2. The term “PDE4D2” includes vertebrate homologs of PDE4D2 family members including, but not limited to mammalian and avian homologs. Representative mammalian homologs of PDE4D2 family members include, but are not limited to murine and human homologs.

As used herein, the terms “PDE4D2 gene” and “recombinant PDE4D2 gene” are used interchangeably and refer to a nucleic acid molecule comprising an open reading frame encoding a PDE4D2 polypeptide, including both exon and (optionally) intron sequences.

As used herein, the terms “PDE4D2 gene product”, “PDE4D2 protein”, “PDE4D2 polypeptide”, and “PDE4D2 peptide” are used interchangeably and refer to peptides having amino acid sequences which are substantially identical to native PDE4D2 amino acid sequences from the organism of interest and which are biologically active in that they comprise all or a part of the amino acid sequence of a PDE4D2 polypeptide, or cross-react with antibodies raised against a PDE4D2 polypeptide, or retain all or some of the biological activity (i.e., catalytic ability and/or dimerization ability) of the native amino acid sequence or protein. Such biological activity can include immunogenicity.

As used herein, the terms “PDE4D2 gene product”, “PDE4D2 protein”, “PDE4D2 polypeptide”, and “PDE4D2 peptide” are used interchangeably and refer to a subtype of the PDE4D2 family. In one embodiment, a PDE4D2 gene product is PDE4D2. In another embodiment, a PDE4D2 gene product comprises the amino acid sequence of SEQ ID NO: 2.

As used herein, the terms “PDE4D2 gene product”, “PDE4D2 protein”, “PDE4D2 polypeptide”, and “PDE4D2 peptide” also include analogs of a PDE4D2 polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or those from other organisms, or can be created synthetically. Those skilled in the art will appreciate that other analogs, as yet undisclosed or undiscovered, can be used to design and/or construct PDE4D2 analogs. There is no need for a “PDE4D2 gene product”, “PDE4D2 protein”, “PDE4D2 polypeptide”, or “PDE4D2 peptide” to comprise all or substantially all of the amino acid sequence of a PDE4D2 polypeptide gene product. Shorter or longer sequences are anticipated to be of use in the presently disclosed subject matter; shorter sequences are herein referred to as “segments”. Thus, the terms “PDE4D2 gene product”, “PDE4D2 protein”, “PDE4D2 polypeptide”, and “PDE4D2 peptide” also include fusion or recombinant PDE4D2 polypeptides and proteins comprising sequences of the presently disclosed subject matter. Methods of preparing such proteins are disclosed herein and are known in the art.

The term “phenotype” refers to the entire physical, biochemical, and physiological makeup of a cell, i.e., having any one trait or any group of traits.

As used herein, the term “polypeptide” refers to any polymer comprising any of the 20 protein amino acids, regardless of its size. Although “protein” is often used in reference to relatively large polypeptides and “peptide” is often used in reference to small polypeptides, usage of these terms in the art overlaps and varies. The term “polypeptide” as used herein refers to peptides, polypeptides, and proteins, unless otherwise noted. As used herein, the terms “protein”, “polypeptide” and “peptide” are used interchangeably herein when referring to a gene product. The term “polypeptide”, and the terms “protein” and “peptide” which are used interchangeably herein, refers to a polymer of amino acids. Exemplary polypeptides include gene products, naturally occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment may comprise a druggable region, and optionally additional amino acids on one or both sides of the druggable region, which additional amino acids may number from 5, 10, 15, 20, 30, 40, 50, or up to 100 or more residues. Further, fragments can include a sub-fragment of a specific region, which sub-fragment retains a function of the region from which it is derived. In another embodiment, a fragment may have immunogenic properties.

The term “polypeptide of the presently disclosed subject matter” refers to a polypeptide comprising the amino acid sequence set forth in SEQ ID NO: 2 or SEQ ID NO: 4, or an equivalent or fragment thereof, i.e., a polypeptide comprising a sequence consisting of, or consisting essentially of, the amino acid sequence set forth in SEQ ID NO: 2 or SEQ ID NO: 4. Polypeptides of the presently disclosed subject matter include polypeptides comprising all or a portion of the amino acid sequence set forth in SEQ ID NO: 2 or SEQ ID NO: 4; the amino acid sequence set forth in SEQ ID NO: 2 or SEQ ID NO: 4 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to SEQ ID NO: 2 or SEQ ID NO: 4; and functional fragments thereof. Polypeptides of the presently disclosed subject matter also include homologs, i.e., orthologs and paralogs, of SEQ ID NO: 2 or SEQ ID NO: 4.

As used herein, the term “primer” refers to a nucleic acid comprising in one embodiment two or more deoxyribonucleotides or ribonucleotides, in another embodiment more than three, in another embodiment more than eight, and in yet another embodiment at least about 20 nucleotides of an exonic or intronic region. In one embodiment, an oligonucleotide is between ten and thirty bases in length.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the presently disclosed subject matter using standard techniques for protein purification in light of the teachings herein. Purity of a polypeptide may be determined by a number of methods known to those of skill in the art, including for example, amino-terminal amino acid sequence analysis, gel electrophoresis, mass-spectrometry analysis and the methods described in the Exemplification section herein.

The terms “recombinant protein” or “recombinant polypeptide” refer to a polypeptide that is produced by recombinant DNA techniques. An example of such techniques includes the case when DNA encoding the expressed protein is inserted into a suitable expression vector that is in turn used to transform a host cell to produce the protein or polypeptide encoded by the DNA.

A “reference sequence” is a defined sequence used as a basis for a sequence comparison; a reference sequence may be a subset of a larger sequence, for example, as a segment of a full-length protein given in a sequence listing such as SEQ ID NO: 2 or SEQ ID NO: 4, or may comprise a complete protein sequence. Generally, a reference sequence is at least 200, 300 or 400 nucleotides in length, frequently at least 600 nucleotides in length, and often at least 800 nucleotides in length (or the protein equivalent if it is shorter or longer in length). Because two proteins may each (1) comprise a sequence (i.e., a portion of the complete protein sequence) that is similar between the two proteins, and (2) may further comprise a sequence that is divergent between the two proteins, sequence comparisons between two (or more) proteins are typically performed by comparing sequences of the two proteins over a “comparison window” to identify and compare local regions of sequence similarity.

The term “regulatory sequence” is a generic term used throughout the specification to refer to polynucleotide sequences, such as initiation signals, enhancers, regulators and promoters, that are necessary or desirable to affect the expression of coding and non-coding sequences to which they are operably linked. Exemplary regulatory sequences are described in Goeddel; Gene Expression Technology: Methods in Enzymology, Academic Press, San Diego, Calif. (1990), and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, i.e., Pho5, the promoters of the yeast α-mating factors, the polyhedron promoter of the baculovirus system and other sequences known to control the expression of genes of prokaryotic or eukaryotic cells or their viruses, and various combinations thereof. The nature and use of such control sequences may differ depending upon the host organism. In prokaryotes, such regulatory sequences generally include promoter, ribosomal binding site, and transcription termination sequences. The term “regulatory sequence” is intended to include, at a minimum, components whose presence may influence expression, and may also include additional components whose presence is advantageous, for example, leader sequences and fusion partner sequences. In certain embodiments, transcription of a polynucleotide sequence is under the control of a promoter sequence (or other regulatory sequence) that controls the expression of the polynucleotide in a cell-type in which expression is intended. It will also be understood that the polynucleotide can be under the control of regulatory sequences that are the same or different from those sequences which control expression of the naturally occurring form of the polynucleotide.

The term “reporter gene” refers to a nucleic acid comprising a nucleotide sequence encoding a protein that is readily detectable either by its presence or activity, including, but not limited to, luciferase, fluorescent protein (i.e., green fluorescent protein), chloramphenicol acetyl transferase, β-galactosidase, secreted placental alkaline phosphatase, β-lactamase, human growth hormone, and other secreted enzyme reporters. Generally, a reporter gene encodes a polypeptide not otherwise produced by the host cell, which is detectable by analysis of the cell(s), i.e., by the direct fluorometric, radioisotopic or spectrophotometric analysis of the cell(s) and preferably without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, β-lactamase, phosphatases, peroxidases, proteases (tissue plasminogen activator or urokinase) and other enzymes whose function may be detected by appropriate chromogenic or fluorogenic substrates known to those skilled in the art or developed in the future.

The term “sequence homology” refers to the proportion of base matches between two nucleic acid sequences or the proportion of amino acid matches between two amino acid sequences. When sequence homology is expressed as a percentage, i.e., 50%, the percentage denotes the proportion of matches over the length of sequence from a desired sequence (i.e., SEQ. ID NO: 1) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail herein.

As used herein, the term “sequencing” refers to determining the ordered linear sequence of nucleotides or amino acids of a DNA, RNA, or protein target sample, using conventional manual or automated laboratory techniques.

The term “small molecule” refers to a compound, which has a molecular weight of less than about 5 kilodalton (kD), less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids, or other organic (carbon containing) or inorganic molecules. Many pharmaceutical companies have extensive libraries of chemical and/or biological mixtures, often fungal, bacterial, or algal extracts, which can be screened with any of the assays of the presently disclosed subject matter. The term “small organic molecule” refers to a small molecule that is often identified as being an organic or medicinal compound, and does not include molecules that are exclusively nucleic acids, peptides, or polypeptides.

The term “soluble” as used herein with reference to a polypeptide of the presently disclosed subject matter or other protein, means that upon expression in cell culture, at least some portion of the polypeptide or protein expressed remains in the cytoplasmic fraction of the cell and does not fractionate with the cellular debris upon lysis and centrifugation of the lysate. Solubility of a polypeptide may be increased by a variety of art recognized methods, including fusion to a heterologous amino acid sequence, deletion of amino acid residues, amino acid substitution (i.e., enriching the sequence with amino acid residues having hydrophilic side chains), and chemical modification (i.e., addition of hydrophilic groups). The solubility of polypeptides may be measured using a variety of art recognized techniques, including, dynamic light scattering to determine aggregation state, UV absorption, centrifugation to separate aggregated from non-aggregated material, and SDS gel electrophoresis (i.e., the amount of protein in the soluble fraction is compared to the amount of protein in the soluble and insoluble fractions combined). When expressed in a host cell, the polypeptides of the presently disclosed subject matter may be at least about 1%, 2%, 5%, 10%, 20%, 30%, 40%, 50%, 60%, 70%, 80%, 90% or more soluble, i.e., at least about 1%, 2%, 5%, 10%, 20%, 30%, 40%, 50%, 60%, 70%, 80%, 90% or more of the total amount of protein expressed in the cell is found in the cytoplasmic fraction. In certain embodiments, a one liter culture of cells expressing a polypeptide of the presently disclosed subject matter will produce at least about 0.1, 0.2, 0.5, 1, 2, 5, 10, 20, 30, 40, 50 milligrams or more of soluble protein. In an exemplary embodiment, a polypeptide of the presently disclosed subject matter is at least about 10% soluble and will produce at least about 1 milligram of protein from a one liter cell culture.

As used herein, the term “space group” refers to the arrangement of symmetry elements of a crystal.

The term “specifically hybridizes” refers to detectable and specific nucleic acid binding. Polynucleotides, oligonucleotides, and nucleic acids of the presently disclosed subject matter selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the presently disclosed subject matter and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

As used herein, the terms “structure coordinates”, “structural coordinates”, and “atomic coordinates” are used interchangeably and refer to coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of coordinates determined by X-ray crystallography is not without standard error. In general, the error in the coordinates tends to be reduced as the resolution is increased, since more experimental diffraction data is available for the model fitting and refinement. Thus, for example, more diffraction data can be collected from a crystal that diffracts to a resolution of 2.8-3.2 Å than from a crystal that diffracts to a lower resolution, such as 3.5 Å. Consequently, the refined structural coordinates will usually be more accurate when fitted and refined using data from a crystal that diffracts to higher resolution. The design of ligands for a PDE4D2 or any other phosphodiesterase depends on the accuracy of the structural coordinates. If the coordinates are not sufficiently accurate, then the design process will be ineffective. In most cases, it is very difficult or impossible to collect sufficient diffraction data to define atomic coordinates precisely when the crystals diffract to a resolution of poorer than 3.5 Å. Thus, in most cases, it is difficult to use X-ray structures in structure-based ligand design when the X-ray structures are based on crystals that diffract to a resolution of poorer than 3.5 Å. However, common experience has shown that crystals diffracting to 2.8-3.5 Å or better can yield X-ray structures with sufficient accuracy to greatly facilitate structure-based drug design. Further improvement in the resolution can further facilitate structure-based design, but the coordinates obtained at 2.8-3.5 Å resolution are generally considered adequate for most purposes.

Also, those of skill in the art will understand that PDE4D2 proteins can adopt different conformations when different ligands are bound. PDE4D2 proteins can adopt different conformations when agonists and antagonists are bound. Subtle variations in the conformation can also occur when different agonists are bound, and when different antagonists are bound. These variations can be difficult or impossible to predict from a single X-ray structure. Generally, structure-based design of PDE4D2 ligands depends to some degree on an understanding of the differences in conformation that occur when agonists and antagonists are bound. Thus, structure-based ligand design is most facilitated by the availability of X-ray structures of complexes with potent agonists as well as potent antagonists.

The terms “stringent conditions” or “stringent hybridization conditions” refer to conditions that promote specific hybridization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, i.e., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt's Solution (0.2% Ficoll, 0.2% Polyvinylpyrrolidone, 0.2% bovine serum albumin) and 200 μg/ml of denatured carrier DNA, i.e., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, i.e., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Ausubel et al., Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in Biochemistry and Molecular Biology-Hybridization With Nucleic Acid Probes, i.e., part I chapter 2 “Overview of principles of hybridization and the strategy of nucleic acid probe assays”, Elsevier, New York; and Tibanyenda, N. et al., Eur. J. Biochem. 139:19 (1984) and Ebel, S. et al., Biochem. 31:12083 (1992).

The term “structural motif”, when used in reference to a polypeptide, refers to a polypeptide that, although it may have different amino acid sequences, may result in a similar structure, wherein by structure is meant that the motif forms generally the same tertiary structure, or that certain amino acid residues within the motif, or alternatively their backbone or side chains (which may or may not include the Cα atoms of the side chains) are positioned in a like relationship with respect to one another in the motif.

As applied to proteins, the term “substantial identity” means that two protein sequences, when optimally aligned, such as by the programs GAP or BESTFIT using default gap weights, typically share at least about 70 percent sequence identity, alternatively at least about 80, 85, 90, 95 percent sequence identity or more. In certain instances, residue positions that are not identical differ by conservative amino acid substitutions, which are described above.

As used herein, the term “substantially pure” refers to a polynucleotide or polypeptide that is substantially free of the sequences and molecules with which it is associated in its natural state, as well as from those molecules used in the isolation procedure. The term “substantially free” refers to that the sample is in one embodiment at least 50%, in another embodiment at least 70%, in another embodiment at least 80%, and in still another embodiment at least 90% free of the sequences and molecules with which is it associated in nature.

As used herein, the term “target cell” refers to a cell, into which it is desired to insert a nucleic acid sequence or polypeptide, or to otherwise effect a modification from conditions known to be present in the unmodified cell. A nucleic acid sequence introduced into a target cell can be of variable length. Additionally, a nucleic acid sequence can enter a target cell as a component of a plasmid or other vector or as a naked sequence.

The term “test compound” refers to a molecule to be tested by one or more screening method(s) as a putative modulator of a polypeptide of the presently disclosed subject matter or other biological entity or process. A test compound is usually not known to bind to a target of interest. The term “control test compound” refers to a compound known to bind to the target (i.e., a known agonist, antagonist, partial agonist or inverse agonist). The term “test compound” does not include a chemical added as a control condition that alters the function of the target to determine signal specificity in an assay. Such control chemicals or conditions include chemicals that 1) nonspecifically or substantially disrupt protein structure (i.e., denaturing agents (i.e., urea or guanidinium), chaotropic agents, sulfhydryl reagents (i.e., dithiothreitol and β-mercaptoethanol), and proteases), 2) generally inhibit cell metabolism (i.e., mitochondrial uncouplers) and 3) non-specifically disrupt electrostatic or hydrophobic interactions of a protein (i.e., high salt concentrations, or detergents at concentrations sufficient to non-specifically disrupt hydrophobic interactions). Further, the term “test compound” also does not include compounds known to be unsuitable for a therapeutic use for a particular indication due to toxicity of the subject. In certain embodiments, various predetermined concentrations of test compounds are used for screening such as 0.01 μM, 0.1 μM, 1.0 μM, and 10.0 μM. Examples of test compounds include, but are not limited to, peptides, nucleic acids, carbohydrates, and small molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay.

The term “therapeutically effective amount” refers to that amount of a modulator, drug, or other molecule that is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “transfection” means the introduction of a nucleic acid, i.e., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell may express a recombinant form of a polypeptide of the presently disclosed subject matter or antisense expression may occur from the transferred gene so that the expression of a naturally occurring form of the gene is disrupted.

The term “transgene” means a nucleic acid sequence, which is partly or entirely heterologous to a transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (i.e., it is inserted at a location which differs from that of the natural gene or its insertion results in a knockout). A transgene may include one or more regulatory sequences and any other nucleic acids, such as introns, that may be necessary for optimal expression.

The term “transgenic animal” refers to any animal, for example, a mouse, rat or other non-human mammal, a bird or an amphibian, in which one or more of the cells of the animal contain heterologous nucleic acid introduced by way of human intervention, such as by transgenic techniques well known in the art. The nucleic acid is introduced into the cell, directly or indirectly, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a protein. However, transgenic animals in which the recombinant gene is silent are also contemplated.

As used herein, the term “unit cell” refers to a basic parallelepiped shaped block. The entire volume of a crystal can be constructed by regular assembly of such blocks. Each unit cell comprises a complete representation of the unit of pattern, the repetition of which builds up the crystal. Thus, the term “unit cell” refers to the fundamental portion of a crystal structure that is repeated infinitely by translation in three dimensions. A unit cell is characterized by three vectors a, b, and c, not located in one plane, which form the edges of a parallelepiped. Angles α, β, and γ define the angles between the vectors: angle α is the angle between vectors b and c; angle β is the angle between vectors a and c; and angle γ is the angle between vectors a and b. The entire volume of a crystal can be constructed by regular assembly of unit cells, each unit cell comprising a complete representation of the unit of pattern, the repetition of which builds up the crystal.

The term “vector” refers to a nucleic acid capable of transporting another nucleic acid to which it has been linked. One type of vector that may be used in accord with the presently disclosed subject matter is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules that, in their vector form are not bound to the chromosome. In the present specification, “plasmid” and “vector” are used interchangeably as the plasmid is the most commonly used form of vector. However, the presently disclosed subject matter is intended to include such other forms of expression vectors which serve equivalent functions and which become known in the art subsequently hereto.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that may vary depending upon the desired properties sought to be obtained by the presently disclosed subject matter.

II. Description of Tables

Table 1 presents data concerning the interfacial interactions in PDE4D2-AMP. Table 1 includes data related to the atoms of individual amino acid residues and AMP that are predicted to be involved in the formation of either hydrogen bonds or van der Waals interactions.

Table 2 presents data concerning the predicted hydrogen bonding and van der Waals interactions that AMP makes with the active site residues of PDE4D2.

Table 3 presents statistics on diffraction data and structure refinement of PDE4D2-AMP. See also Example 2.

Table 4 presents atomic structure coordinate data obtained from X-ray diffraction from the catalytic domain of PDE4D2 in complex with AMP.

Table 5 presents atomic structure coordinate data obtained from X-ray diffraction from unligated PDE4D2 (polypeptide only without ligand).

III. Production of PDE4D2 Catalytic Domain Polypeptides

The native and mutated PDE4D2 polypeptides, and fragments thereof, of the presently disclosed subject matter can be chemically synthesized in whole or part using techniques that are well known in the art (see i.e., Creighton, (1983) Proteins: Structures and Molecular Principles, W.H. Freeman & Co., New York, incorporated herein in its entirety). Alternatively, methods which are well known to those skilled in the art can be used to construct expression vectors containing a partial or the entire native or mutated PDE4D2 polypeptide coding sequence and appropriate transcriptional/translational control signals. These methods include in vitro recombinant DNA techniques, synthetic techniques, and in vivo recombination/genetic recombination (see i.e., the techniques described throughout Sambrook et al., (1989) Molecular Cloning: A Laboratory Manual, Cold Spring Harbor Laboratory, New York, and Ausubel et al., (1989) Current Protocols in Molecular Biology, Greene Publishing Associates and Wiley Interscience, New York, both incorporated herein in their entirety).

Some of the functions of a domain within the full-length protein are preserved when that particular domain is isolated from the remainder of the protein. Using conventional protein chemistry techniques, a modular domain can sometimes be separated from the parent protein. Using conventional molecular biology techniques, each domain can usually be separately expressed with its original function intact or, as discussed herein below, chimeras comprising two different proteins can be constructed, wherein the chimeras retain the properties of the individual functional domains of the respective phosphodiesterases from which the chimeras were generated.

As described herein, the catalytic domain of a PDE4D2 can be expressed, crystallized, and its three dimensional structure determined with a ligand bound as disclosed in the presently disclosed subject matter. Additionally, the three dimensional structure that is determined can be used to identify new ligands and computational methods can be used to design ligands to its catalytic domain.

A variety of host-expression vector systems can be utilized to express a PDE4D2 coding sequence. These include, but are not limited to microorganisms such as bacteria transformed with recombinant bacteriophage DNA, plasmid DNA or cosmid DNA expression vectors containing a PDE4D2 coding sequence; yeast transformed with recombinant yeast expression vectors containing a PDE4D2 coding sequence; insect cell systems infected with recombinant virus expression vectors (i.e., baculovirus) containing a PDE4D2 coding sequence; plant cell systems infected with recombinant virus expression vectors (i.e., cauliflower mosaic virus, CaMV; tobacco mosaic virus, TMV; or transformed with recombinant plasmid expression vectors (i.e., Ti plasmid) containing a PDE4D2 coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter), and the like can be used. When cloning in insect cell systems, promoters such as the baculovirus polyhedrin promoter can be used. When cloning in plant cell systems, promoters derived from the genome of plant cells, such as heat shock promoters; the promoter for the small subunit of ribulose bisphosphate carboxylase (RUBISCO); the promoter for the chlorophyll a/b binding protein; or from plant viruses (i.e., the 35S RNA promoter of CaMV; the coat protein promoter of TMV) can be used. When cloning in mammalian cell systems, promoters derived from the genome of mammalian cells (i.e., metallothionein promoter) or from mammalian viruses (i.e., the adenovirus late promoter; the vaccinia virus 7.5K promoter) can be used. In each of these systems, one of ordinary skill in the art will appreciate that other promoters can be used, and as such, the list presented is not intended to be exhaustive.

IV. Analysis of Protein Properties

IV.A. Analysis of Proteins by X-Ray Crystallography Generally

IV.A.1. X-Ray Structure Determination

Exemplary methods for obtaining the three dimensional structure of the crystalline form of a molecule or complex are described herein and, in view of this specification, variations on these methods will be apparent to those skilled in the art (see Ducruix and Geige 1992, Crystallization of Nucleic Acids and Proteins: A Practical Approach, IRL Press, Oxford, England).

A variety of methods involving x-ray crystallography are contemplated by the presently disclosed subject matter. For example, the presently disclosed subject matter contemplates producing a crystallized polypeptide of the presently disclosed subject matter, or a fragment thereof, by: (a) introducing into a host cell an expression vector comprising a nucleic acid encoding for a polypeptide of the presently disclosed subject matter, or a fragment thereof; (b) culturing the host cell in a cell culture medium to express the polypeptide or fragment; (c) isolating the polypeptide or fragment from the cell culture; and (d) crystallizing the polypeptide or fragment thereof. Alternatively, the presently disclosed subject matter contemplates determining the three dimensional structure of a crystallized polypeptide of the presently disclosed subject matter, or a fragment thereof, by: (a) crystallizing a polypeptide of the presently disclosed subject matter, or a fragment thereof, such that the crystals will diffract x-rays to a resolution of 3.5 Å or better; and (b) analyzing the polypeptide or fragment by x-ray diffraction to determine the three-dimensional structure of the crystallized polypeptide.

X-ray crystallography techniques generally require that the protein molecules be available in the form of a crystal. Crystals may be grown from a solution containing a purified polypeptide of the presently disclosed subject matter, or a fragment thereof (i.e., a stable domain), by a variety of conventional processes. These processes include, for example, batch, liquid, bridge, dialysis, vapour diffusion (i.e., hanging drop or sitting drop methods). See e.g., McPherson, 1982, Preparation and Analysis of Protein Crystals, John Wiley, New York; McPherson, 1990, Eur. J. Biochem. 189: 1-23; Weber. 1991, Adv. Protein Chem. 41: 1-36.

In certain embodiments, native crystals of the presently disclosed subject matter may be grown by adding precipitants to the concentrated solution of the polypeptide. The precipitants are added at a concentration just below that necessary to precipitate the protein. Water may be removed by controlled evaporation to produce precipitating conditions, which are maintained until crystal growth ceases.

The formation of crystals is dependent on a number of different parameters, including pH, temperature, protein concentration, the nature of the solvent and precipitant, as well as the presence of added ions or ligands to the protein. In addition, the sequence of the polypeptide being crystallized will have a significant affect on the success of obtaining crystals. Many routine crystallization experiments may be needed to screen all these parameters for the few combinations that might give crystal suitable for x-ray diffraction analysis (see e.g., Jancarik, J & Kim, S. H., J. Appl. Cryst. 1991 24: 409-411).

Crystallization robots may automate and speed up the work of reproducibly setting up large number of crystallization experiments. Once some suitable set of conditions for growing the crystal are found, variations of the condition may be systematically screened in order to find the set of conditions which allows the growth of sufficiently large, single, well ordered crystals. In certain instances, a polypeptide of the presently disclosed subject matter is co-crystallized with a compound that stabilizes the polypeptide.

A number of methods are available to produce suitable radiation for x-ray diffraction. For example, x-ray beams may be produced by synchrotron rings where electrons (or positrons) are accelerated through an electromagnetic field while traveling at close to the speed of light. Because the admitted wavelength may also be controlled, synchrotrons may be used as a tunable x-ray source (Hendrickson W A, Trends Biochem Sci 2000 December; 25(12): 637-43). For less conventional Laue diffraction studies, polychromatic x-rays covering a broad wavelength window are used to observe many diffraction intensities simultaneously (Stoddard B L, Curr. Opin. Struct Biol 1998 October; 8(5): 612-8). Neutrons may also be used for solving protein crystal structures (Gutberlet T, Heinemann U & Steiner M, Acta Crystallogr D 2001, 57: 349-54).

Before data collection commences, a protein crystal may be frozen to protect it from radiation damage. A number of different cryo-protectants may be used to assist in freezing the crystal, such as methyl pentanediol (MPD), isopropanol, ethylene glycol, glycerol, formate, citrate, mineral oil, or a low-molecular-weight polyethylene glycol (PEG). The presently disclosed subject matter contemplates a composition comprising a polypeptide of the presently disclosed subject matter and a cryo-protectant. As an alternative to freezing the crystal, the crystal may also be used for diffraction experiments performed at temperatures above the freezing point of the solution. In these instances, the crystal may be protected from drying out by placing it in a narrow capillary of a suitable material (generally glass or quartz) with some of the crystal growth solution included in order to maintain vapour pressure.

X-ray diffraction results may be recorded by a number of ways know to one of skill in the art. Examples of area electronic detectors include charge coupled device detectors, multi-wire area detectors and phosphoimager detectors (Amemiya, Y, 1997, Methods in Enzymology, Vol. 276, Academic Press, San Diego, Calif., United States of America, pp. 233-243; Westbrook E M & Naday I, 1997, Methods in Enzymology, Vol. 276, Academic Press, San Diego, Calif., United States of America, pp. 244-268; Kahn R & Fourme R, 1997, Methods in Enzymology, Vol. 276, Academic Press, San Diego, Calif., United States of America, pp. 268-286).

A suitable system for laboratory data collection might include a Bruker AXS Proteum R system, equipped with a copper rotating anode source, Confocal MAX-FLUX™ optics and a SMART 6000 charge coupled device detector. Collection of x-ray diffraction patterns are well documented by those skilled in the art (see i.e., Ducruix and Geige, 1992, Crystallization of Nucleic Acids and Proteins: A Practical Approach, IRL Press, Oxford, England).

The theory behind diffraction by a crystal upon exposure to x-rays is well known. Because phase information is not directly measured in the diffraction experiment, and is needed to reconstruct the electron density map, methods that can recover this missing information are required. One method of solving structures ab initio are the real/reciprocal space cycling techniques. Suitable real/reciprocal space cycling search programs include shake-and-bake (Weeks C M, DeTitta G T, Hauptman H A, Thuman P, Miller R, Acta Crystallogr A 1994; 50: 210-20).

Other methods for deriving phases may also be needed. These techniques generally rely on the idea that if two or more measurements of the same reflection are made where strong, measurable, differences are attributable to the characteristics of a small subset of the atoms alone, then the contributions of other atoms can be, to a first approximation, ignored, and positions of these atoms may be determined from the difference in scattering by one of the above techniques. Knowing the position and scattering characteristics of those atoms, one may calculate what phase the overall scattering must have had to produce the observed differences.

One version of this technique is isomorphous replacement technique, which requires the introduction of new, well ordered, x-ray scatterers into the crystal. These additions are usually heavy metal atoms, (so that they make a significant difference in the diffraction pattern); and if the additions do not change the structure of the molecule or of the crystal cell, the resulting crystals should be isomorphous. Isomorphous replacement experiments are usually performed by diffusing different heavy-metal metals into the channels of a pre-existing protein crystal. Growing the crystal from protein that has been soaked in the heavy atom is also possible (Petsko G A, 1985, Methods in Enzymology, Vol. 114, Academic Press, Orlando, Fla., United States of America, pp. 147-156). Alternatively, the heavy atom may also be reactive and attached covalently to exposed amino acid side chains (such as the sulfur atom of cysteine) or it may be associated through non-covalent interactions. It is sometimes possible to replace endogenous light metals in metallo-proteins with heavier ones, i.e., zinc by mercury, or calcium by samarium (Petsko G A, 1985, Methods in Enzymology, Vol. 114, Academic Press, Orlando, Fla., United States of America, pp. 147-156). Exemplary sources for such heavy compounds include, without limitation, sodium bromide, sodium selenate, trimethyl lead acetate, mercuric chloride, methyl mercury acetate, platinum tetracyanide, platinum tetrachloride, nickel chloride, and europium chloride.

A second technique for generating differences in scattering involves the phenomenon of anomalous scattering. X-rays that cause the displacement of an electron in an inner shell to a higher shell are subsequently rescattered, but there is a time lag that shows up as a phase delay. This phase delay is observed as a (generally quite small) difference in intensity between reflections known as Friedel mates that would be identical if no anomalous scattering were present. A second effect related to this phenomenon is that differences in the intensity of scattering of a given atom will vary in a wavelength dependent manner, given rise to what are known as dispersive differences. In principle anomalous scattering occurs with all atoms, but the effect is strongest in heavy atoms, and may be maximized by using x-rays at a wavelength where the energy is equal to the difference in energy between shells. The technique therefore requires the incorporation of some heavy atom much as is needed for isomorphous replacement, although for anomalous scattering a wider variety of atoms are suitable, including lighter metal atoms (copper, zinc, iron) in metallo-proteins. One method for preparing a protein for anomalous scattering involves replacing the methionine residues in whole or in part with selenium containing seleno-methionine. Soaks with halide salts such as bromides and other non-reactive ions may also be effective (Dauter Z, Li M, Wlodawer A., Acta Crystallogr D 2001; 57: 239-49).

In another process, known as multiple anomalous scattering or MAD, two to four suitable wavelengths of data are collected. (Hendrickson W A & Ogata C M, 1997, Methods in Enzymology, Vol. 276, San Diego, Calif., United States of America, pp. 494-523). Phasing by various combinations of single and multiple isomorphous and anomalous scattering are possible too. For example, SIRAS (single isomorphous replacement with anomalous scattering) utilizes both the isomorphous and anomalous differences for one derivative to derive phases. More traditionally, several different heavy atoms are soaked into different crystals to get sufficient phase information from isomorphous differences while ignoring anomalous scattering, in the technique known as multiple isomorphous replacement (MIR) (Petsko G A, 1985, Methods in Enzymology, Vol. 114, Academic Press, Orlando, Fla., United States of America, pp. 147-156).

Additional restraints on the phases may be derived from density modification techniques. These techniques use either generally known features of electron density distribution or known facts about that particular crystal to improve the phases. For example, because protein regions of the crystal scatter more strongly than solvent regions, solvent flattening/flipping may be used to adjust phases to make solvent density a uniform flat value (Zhang K Y J, Cowtan K, & Main P, 1997, Methods in Enzymology, Vol. 277, Academic Press, Orlando, Fla., United States of America, pp. 53-64). If more than one molecule of the protein is present in the asymmetric unit, the fact that the different molecules should be virtually identical may be exploited to further reduce phase error using non-crystallographic symmetry averaging (Villieux F M D & Read R J, 1997, Methods in Enzymology, Vol. 277, Academic Press, Orlando, Fla., United States of America, pp. 18-52). Suitable programs for performing these processes include DM and other programs of the CCP4 suite (Collaborative Computational Project, Number 4, 1994, Acta Cryst D50: 760-763) and CNX.

The unit cell dimensions, symmetry, vector amplitude and derived phase information can be used in a Fourier transform function to calculate the electron density in the unit cell, i.e., to generate an experimental electron density map. This may be accomplished using programs of the CNX or CCP4 packages. The resolution is measured in Ångstrom (Å) units, and is closely related to how far apart two objects need to be before they can be reliably distinguished. The smaller this number is, the higher the resolution and therefore the greater the amount of detail that can be seen. In alternative embodiments, crystals of the presently disclosed subject matter diffract x-rays to a resolution of better than about 4.0, 3.5, 3.0, 2.5, 2.0, 1.5, 1.0, 0.5 Å, or better.

As used herein, the term “modeling” includes the quantitative and qualitative analysis of molecular structure and/or function based on atomic structural information and interaction models. The term “modeling” includes conventional numeric-based molecular dynamic and energy minimization models, interactive computer graphic models, modified molecular mechanics models, distance geometry and other structure-based constraint models.

Model building may be accomplished by either the crystallographer using a computer graphics program such as TURBO or O (Jones T A et al., 1991, Acta Crystallogr. A47: 100-119) or, under suitable circumstances, by using a fully automated model building program, such as wARP (Perrakis A, Morris R, & Lamzin, V S, May 1999, Nature Structural Biology 6: 458-463) or MAID (Levitt D G, Acta Crystallogr. D 2001 57: 1013-9). This structure may be used to calculate model-derived diffraction amplitudes and phases. The model-derived and experimental diffraction amplitudes may be compared and the agreement between them can be described by a parameter referred to as R-factor. A high degree of correlation in the amplitudes corresponds to a low R-factor value, with 0.0 representing exact agreement and 0.59 representing a completely random structure. Because the R-factor may be lowered by introducing more free parameters into the model, an unbiased, cross-correlated version of the R-factor known as the R-free gives a more objective measure of model quality. For the calculation of this parameter a subset of reflections (generally around 10%) are set aside at the beginning of the refinement and not used as part of the refinement target. These reflections are then compared to those predicted by the model (Kleywegt G J & Brunger A T, Structure 1996 4(8): 897-904).

The model may be improved using computer programs that maximize the probability that the observed data was produced from the predicted model, while simultaneously optimizing the model geometry. For example, the CNX program may be used for model refinement, as can the XPLOR program (Murshudov G N, Vagin A A, & Dodson E J, 1997, Acta Cryst. D Biol Crystallogr 53: 247-255). In order to maximize the convergence radius of refinement, simulated annealing refinement using torsion angle dynamics may be employed in order to reduce the degrees of freedom of motion of the model (Adams P D, Pannu N S, Read R J, Brunger A T, 1997, Proc Natl Acad Sci USA 94(10): 5018-23). Where experimental phase information is available (i.e., where MAD data was collected) Hendrickson-Lattman phase probability targets can be employed. Isotropic or anisotropic domain, group or individual temperature factor refinement, may be used to model variance of the atomic position from its mean. Well-defined peaks of electron density not attributable to protein atoms are generally modeled as water molecules. Water molecules may be found by manual inspection of electron density maps, or with automatic water picking routines. Additional small molecules, including ions, cofactors, buffer molecules, or substrates may be included in the model if sufficiently unambiguous electron density is observed in a map.

In general, the R-free is rarely as low as 0.15 and may be as high as 0.35 or greater for a reasonably well-determined protein structure. The residual difference is a consequence of approximations in the model (inadequate modeling of residual structure in the solvent, modeling atoms as isotropic Gaussian spheres, assuming all molecules are identical rather than having a set of discrete conformers, etc.) and errors in the data (Lattman E E, 1996, Proteins 25: i-ii). In refined structures at high resolution, there are usually no major errors in the orientation of individual residues, and the estimated errors in atomic positions are usually around 0.1-0.2 up to 0.3 Å.

The three dimensional structure of a new crystal may be modeled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases may then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman E, 1985, Methods in Enzymology, Vol. 115, pp. 55-77; Rossmann M G (ed.), 1972, The Molecular Replacement Method, Gordon & Breach, New York, N.Y., United States of America).

Commonly used computer software packages for molecular replacement are CNX, X-PLOR (Brunger 1992, Nature 355: 472-475), AMoRE (Navaza, 1994, Acta Crystallogr. A50:157-163), the CCP4 package, the MERLOT package (Fitzgerald P M D, 1988 J. Appl. Cryst., Vol. 21, pp. 273-278) and XTALVIEW (McCree et al., 1992, J. Mol. Graphics 10: 44-46). The quality of the model may be analyzed using a program such as PROCHECK or 3D-Profiler (Laskowski et al., 1993, J. Appl. Cryst 26:283-291; Luthy R et al., 1992, Nature 356: 83-85; and Bowie J U et al., 1991, Science 253: 164-170).

Homology modeling (also known as comparative modeling or knowledge-based modeling) methods may also be used to develop a three dimensional model from a polypeptide sequence based on the structures of known proteins. The method utilizes a computer model of a known protein, a computer representation of the amino acid sequence of the polypeptide with an unknown structure, and standard computer representations of the structures of amino acids. This method is well known to those skilled in the art (Greer, 1985, Science 228: 1055; Bundell et al., 1988, Eur. J. Biochem. 172: 513; Knighton et al., 1992, Science 258: 130-135). Computer programs that can be used in homology modeling are QUANTA and the Homology module in the Insight II modeling package distributed by Molecular Simulations Inc. (now part of Accelrys Inc., San Diego, Calif., United States of America), or MODELLER (Rockefeller University, New York, N.Y., United States of America; www.iucr.ac.uk/sinris-top/logical/prg-modeller.html).

Once a homology model has been generated it is analyzed to determine its correctness. A computer program available to assist in this analysis is the Protein Health module in QUANTA that provides a variety of tests. Other programs that provide structure analysis along with output include PROCHECK and 3D-Profiler (Luthy R et al., 1992, Nature 356: 83-85; and Bowie et al., 1991, Science 253: 164-170). Once any irregularities have been resolved, the entire structure may be further refined.

Other molecular modeling techniques may also be employed in accordance with presently disclosed subject matter. See e.g., Cohen et al., 1990, J. Med. Chem. 33: 883-894; Navia M A & Murcko M A, 1992, Current Opinions in Structural Biology 2: 202-210.

Under suitable circumstances, the entire process of solving a crystal structure may be accomplished in an automated fashion by a system such as ELVES (http://ucxray.berkeley.edu/˜jamesh/elves/index.html) with little or no user intervention.

IV.A.2. X-Ray Structure

The presently disclosed subject matter provides methods for determining some or all of the structural coordinates for amino acids of a polypeptide of the presently disclosed subject matter, or a complex thereof.

In another aspect, the presently disclosed subject matter provides methods for identifying a druggable region of a polypeptide of the presently disclosed subject matter. For example, one such method includes: (a) obtaining crystals of a polypeptide of the presently disclosed subject matter or a fragment thereof such that the three dimensional structure of the crystallized protein can be determined to a resolution of 3.5 Å or better; (b) determining the three dimensional structure of the crystallized polypeptide or fragment using x-ray diffraction; and (c) identifying a druggable region of a polypeptide of the presently disclosed subject matter based on the three-dimensional structure of the polypeptide or fragment.

A three dimensional structure of a molecule or complex may be described by the set of atoms that best predict the observed diffraction data (that is, which possesses a minimal R value). Files may be created for the structure that defines each atom by its chemical identity, spatial coordinates in three dimensions, root mean squared deviation from the mean observed position and fractional occupancy of the observed position.

Those of skill in the art understand that a set of structure coordinates for an protein, complex or a portion thereof, is a relative set of points that define a shape in three dimensions. Thus, it is possible that an entirely different set of coordinates could define a similar or identical shape. Moreover, slight variations in the individual coordinates may have little affect on overall shape. Such variations in coordinates may be generated because of mathematical manipulations of the structure coordinates. For example, structure coordinates could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little affect on overall shape. If such variations are within an acceptable standard error as compared to the original coordinates, the resulting three-dimensional shape is considered to be structurally equivalent. It should be noted that slight variations in individual structure coordinates of a polypeptide of the presently disclosed subject matter or a complex thereof would not be expected to significantly alter the nature of modulators that could associate with a druggable region thereof. Thus, for example, a modulator that bound to the active site of a polypeptide of the presently disclosed subject matter would also be expected to bind to or interfere with another active site whose structure coordinates define a shape that falls within the acceptable error.

A crystal structure of the presently disclosed subject matter may be used to make a structural or computer model of the polypeptide, complex, or portion thereof. A model may represent the secondary, tertiary, and/or quaternary structure of the polypeptide, complex, or portion. The configurations of points in space derived from structure coordinates according to the presently disclosed subject matter can be visualized as, for example, a holographic image, a stereodiagram, a model, or a computer-displayed image, and the presently disclosed subject matter thus includes such images, diagrams, or models.

IV.A.3. Structural Equivalents

Various computational analyses can be used to determine whether a molecule or the active site portion thereof is structurally equivalent with respect to its three-dimensional structure, to all or part of a structure of a polypeptide of the presently disclosed subject matter or a portion thereof.

For the purpose of presently disclosed subject matter, any molecule or complex or portion thereof, that has a root mean square deviation of conserved residue backbone atoms (N, Cα, C, O) of less than about 1.75 Å, when superimposed on the relevant backbone atoms described by the reference structure coordinates of a polypeptide of the presently disclosed subject matter, is considered “structurally equivalent” to the reference molecule. That is to say, the crystal structures of those portions of the two molecules are substantially identical, within acceptable error. Alternatively, the root mean square deviation may be is less than about 1.50, 1.40, 1.25, 1.0, 0.75, 0.5 or 0.35 Å.

The term “root mean square deviation” is understood in the art and means the square root of the arithmetic mean of the squares of the deviations. It is a way to express the deviation or variation from a trend or object.

In another aspect, the presently disclosed subject matter provides a scalable three-dimensional configuration of points, at least a portion of said points, and preferably all of said points, derived from structural coordinates of at least a portion of a polypeptide of the presently disclosed subject matter and having a root mean square deviation from the structure coordinates of the polypeptide of the presently disclosed subject matter of less than 1.50, 1.40, 1.25, 1.0, 0.75, 0.5 or 0.35 Å. In certain embodiments, the portion of a polypeptide of the presently disclosed subject matter is 25%, 33%, 50%, 66%, 75%, 85%, 90% or 95% or more of the amino acid residues contained in the polypeptide.

In another aspect, the presently disclosed subject matter provides a molecule or complex including a druggable region of a polypeptide of the presently disclosed subject matter, the druggable region being defined by a set of points having a root mean square deviation of less than about 1.75 Å from the structural coordinates for points representing (a) the backbone atoms of the amino acids contained in a druggable region of a polypeptide of the presently disclosed subject matter, (b) the side chain atoms (and optionally the Cα atoms) of the amino acids contained in such druggable region, or (c) all the atoms of the amino acids contained in such druggable region. In certain embodiments, only a portion of the amino acids of a druggable region may be included in the set of points, such as 25%, 33%, 50%, 66%, 75%, 85%, 90% or 95% or more of the amino acid residues contained in the druggable region. In certain embodiments, the root mean square deviation may be less than 1.50, 1.40, 1.25, 1.0, 0.75, 0.5, or 0.35 Å. In still other embodiments, instead of a druggable region, a stable domain, fragment, or structural motif is used in place of a druggable region.

IV.A.4. Machine Displays and Machine Readable Storage Media

The presently disclosed subject matter provides a machine-readable storage medium including a data storage material encoded with machine readable data which, when using a machine programmed with instructions for using said data, displays a graphical three-dimensional representation of any of the molecules or complexes, or portions thereof, of presently disclosed subject matter. In another embodiment, the graphical three-dimensional representation of such molecule, complex or portion thereof includes the root mean square deviation of certain atoms of such molecule by a specified amount, such as the backbone atoms by less than 0.8 Å. In another embodiment, a structural equivalent of such molecule, complex, or portion thereof, may be displayed. In another embodiment, the portion may include a druggable region of the polypeptide of the presently disclosed subject matter.

According to one embodiment, the presently disclosed subject matter provides a computer for determining at least a portion of the structure coordinates corresponding to x-ray diffraction data obtained from a molecule or complex, wherein said computer includes: (a) a machine-readable data storage medium comprising a data storage material encoded with machine-readable data, wherein said data comprises at least a portion of the structural coordinates of a polypeptide of the presently disclosed subject matter; (b) a machine-readable data storage medium comprising a data storage material encoded with machine-readable data, wherein said data comprises x-ray diffraction data from said molecule or complex; (c) a working memory for storing instructions for processing said machine-readable data of (a) and (b); (d) a central-processing unit coupled to said working memory and to said machine-readable data storage medium of (a) and (b) for performing a Fourier transform of the machine readable data of (a) and for processing said machine readable data of (b) into structure coordinates; and (e) a display coupled to said central-processing unit for displaying said structure coordinates of said molecule or complex. In certain embodiments, the structural coordinates displayed are structurally equivalent to the structural coordinates of a polypeptide of the presently disclosed subject matter.

In an alternative embodiment, the machine-readable data storage medium includes a data storage material encoded with a first set of machine readable data which includes the Fourier transform of the structure coordinates of a polypeptide of the presently disclosed subject matter or a portion thereof, and which, when using a machine programmed with instructions for using said data, can be combined with a second set of machine readable data including the x-ray diffraction pattern of a molecule or complex to determine at least a portion of the structure coordinates corresponding to the second set of machine readable data.

For example, a system for reading a data storage medium may include a computer including a central processing unit (CPU), a working memory which can be, i.e., random access memory (RAM) or “core” memory, mass storage memory (such as one or more disk drives or CD-ROM drives), one or more display devices (i.e., cathode-ray tube (“CRT”) displays, light emitting diode (LED) displays, liquid crystal displays (LCDs), electroluminescent displays, vacuum fluorescent displays, field emission displays (FEDs), plasma displays, projection panels, etc.), one or more user input devices (i.e., keyboards, microphones, mice, touch screens, etc.), one or more input lines, and one or more output lines, all of which are interconnected by a conventional bidirectional system bus. The system may be a stand-alone computer, or may be networked (i.e., through local area networks, wide area networks, intranets, extranets, or the internet) to other systems (i.e., computers, hosts, servers, etc.). The system may also include additional computer controlled devices such as consumer electronics and appliances.

Input hardware may be coupled to the computer by input lines and may be implemented in a variety of ways. Machine-readable data of presently disclosed subject matter may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of an active site of presently disclosed subject matter using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of presently disclosed subject matter. Such programs are discussed in reference to the computational methods of drug discovery as described herein. References to components of the hardware system are included as appropriate throughout the following description of the data storage medium.

Machine-readable storage devices useful in the presently disclosed subject matter include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (i.e., drives, controllers, power supplies, etc.) as well as any necessary media (i.e., disks, flash cards, etc.) to enable the storage of data.

In one embodiment, the presently disclosed subject matter contemplates a computer readable storage medium comprising structural data, wherein the data include the identity and three-dimensional coordinates of a polypeptide of the presently disclosed subject matter or portion thereof. In another aspect, the presently disclosed subject matter contemplates a database comprising the identity and three-dimensional coordinates of a polypeptide of the presently disclosed subject matter or a portion thereof. Alternatively, the presently disclosed subject matter contemplates a database comprising a portion or all of the atomic coordinates of a polypeptide of the presently disclosed subject matter or portion thereof.

IV.A.5. Structurally Similar Molecules and Complexes

Structural coordinates for a polypeptide of the presently disclosed subject matter can be used to aid in obtaining structural information about another molecule or complex. This method of the presently disclosed subject matter allows determination of at least a portion of the three-dimensional structure of molecules or molecular complexes that contain one or more structural features that are similar to structural features of a polypeptide of the presently disclosed subject matter. Similar structural features can include, for example, regions of amino acid identity, conserved active site or binding site motifs, and similarly arranged secondary structural elements (i.e., α helices and β sheets). Many of the methods described above for determining the structure of a polypeptide of the presently disclosed subject matter may be used for this purpose as well.

For the presently disclosed subject matter, a “structural homolog” is a polypeptide that contains one or more amino acid substitutions, deletions, additions, or rearrangements with respect to the amino acid sequence of SEQ ID NOs: 2 or 4 or other polypeptide of the presently disclosed subject matter, but that, when folded into its native conformation, exhibits or is reasonably expected to exhibit at least a portion of the tertiary (three-dimensional) structure of the polypeptide encoded by SEQ ID NOs: 2 or 4 or such other polypeptide of the presently disclosed subject matter. For example, structurally homologous molecules can contain deletions or additions of one or more contiguous or noncontiguous amino acids, such as a loop or a domain. Structurally homologous molecules also include modified polypeptide molecules that have been chemically or enzymatically derivatized at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

By using molecular replacement, all or part of the structure coordinates of a polypeptide of the presently disclosed subject matter can be used to determine the structure of a crystallized molecule or complex whose structure is unknown more quickly and efficiently than attempting to determine such information ab initio. For example, in one embodiment presently disclosed subject matter provides a method of utilizing molecular replacement to obtain structural information about a molecule or complex whose structure is unknown including: (a) crystallizing the molecule or complex of unknown structure; (b) generating an x-ray diffraction pattern from said crystallized molecule or complex; and (c) applying at least a portion of the structure coordinates for a polypeptide of the presently disclosed subject matter to the x-ray diffraction pattern to generate a three-dimensional electron density map of the molecule or complex whose structure is unknown.

In another aspect, the presently disclosed subject matter provides a method for generating a preliminary model of a molecule or complex whose structure coordinates are unknown, by orienting and positioning the relevant portion of a polypeptide of the presently disclosed subject matter within the unit cell of the crystal of the unknown molecule or complex so as best to account for the observed x-ray diffraction pattern of the crystal of the molecule or complex whose structure is unknown.

Structural information about a portion of any crystallized molecule or complex that is sufficiently structurally similar to a portion of a polypeptide of the presently disclosed subject matter may be resolved by this method. In addition to a molecule that shares one or more structural features with a polypeptide of the presently disclosed subject matter, a molecule that has similar bioactivity, such as the same catalytic activity, substrate specificity or ligand binding activity as a polypeptide of the presently disclosed subject matter, may also be sufficiently structurally similar to a polypeptide of the presently disclosed subject matter to permit use of the structure coordinates for a polypeptide of the presently disclosed subject matter to solve its crystal structure.

In another aspect, the method of molecular replacement is utilized to obtain structural information about a complex containing a polypeptide of the presently disclosed subject matter, such as a complex between a modulator and a polypeptide of the presently disclosed subject matter (or a domain, fragment, ortholog, homolog etc. thereof). In certain instances, the complex includes a polypeptide of the presently disclosed subject matter (or a domain, fragment, ortholog, homolog etc. thereof) co-complexed with a modulator. For example, in one embodiment, the presently disclosed subject matter contemplates a method for making a crystallized complex comprising a polypeptide of the presently disclosed subject matter, or a fragment thereof, and a compound having a molecular weight of less than 5 kDa, the method comprising: (a) crystallizing a polypeptide of the presently disclosed subject matter such that the crystals will diffract x-rays to a resolution of 3.5 Å or better; and (b) soaking the crystal in a solution comprising the compound having a molecular weight of less than 5 kDa, thereby producing a crystallized complex comprising the polypeptide and the compound.

Using homology modeling, a computer model of a structural homolog or other polypeptide can be built or refined without crystallizing the molecule. For example, in another aspect, the presently disclosed subject matter provides a computer-assisted method for homology modeling a structural homolog of a polypeptide of the presently disclosed subject matter including: aligning the amino acid sequence of a known or suspected structural homolog with the amino acid sequence of a polypeptide of the presently disclosed subject matter and incorporating the sequence of the homolog into a model of a polypeptide of the presently disclosed subject matter derived from atomic structure coordinates to yield a preliminary model of the homolog; subjecting the preliminary model to energy minimization to yield an energy minimized model; remodeling regions of the energy minimized model where stereochemistry restraints are violated to yield a final model of the homolog.

In another embodiment, the presently disclosed subject matter contemplates a method for determining the crystal structure of a homolog of a polypeptide having SEQ ID NO: 2 or SEQ ID NO: 4, or equivalent thereof, the method comprising: (a) providing the three dimensional structure of a crystallized polypeptide having SEQ ID NO: 2 or SEQ ID NO: 4, or a fragment thereof; (b) obtaining crystals of a homologous polypeptide comprising an amino acid sequence that is at least 80% identical to the amino acid sequence set forth in SEQ ID NO: 2 or SEQ ID NO: 4 such that the three dimensional structure of the crystallized homologous polypeptide may be determined to a resolution of 3.5 Å or better; and (c) determining the three dimensional structure of the crystallized homologous polypeptide by x-ray crystallography based on the atomic coordinates of the three dimensional structure provided in step (a). In certain instances of the foregoing method, the atomic coordinates for the homologous polypeptide have a root mean square deviation from the backbone atoms of the polypeptide having SEQ ID NO: 2 or SEQ ID NO: 4, or a fragment thereof, of not more than 1.5 Å for all backbone atoms shared in common with the homologous polypeptide and the polypeptide having SEQ ID NO: 2 or SEQ ID NO: 4, or a fragment thereof.

IV.2. Formation of PDE4D2 Catalytic Domain-Ligand Crystals

The presently disclosed subject matter provides crystals of PDE4D2 catalytic domain (CD) in complex with the ligand. In one embodiment, the PDE4D2 catalytic domain polypeptide used to produce crystals has the amino acid sequence shown in SEQ ID NO: 4. The crystals were obtained using the methodology disclosed in the Examples. Briefly, the crystals were grown by vapor diffusion against a well buffer of 50 mM HEPES (pH 7.5), 15% PEG3350, 25% ethylene glycol, 5% methanol, and 5% DMSO at 4° C. The protein drop was prepared by mixing 10 mM cAMP and 0.4 mM zinc sulfate with 15 mg/mL PDE4D2 in a storage buffer of 50 mM NaCl, 20 mM Tris-HCl (pH 7.5), and 1 mM β-mercaptoethanol for the crystallization. To saturate the cAMP binding, the crystals were soaked in a buffer of 50 mM HEPES (pH 7.5), 20% PEG3350, 25% ethylene glycol, 0.4 mM zinc sulfate, and 50 mM cAMP at room temperature for 5 hours and then immediately dipped into liquid nitrogen. The PDE4D2 crystals, which can be native or derivative crystals, have a space group symmetry P2₁2₁2₁. In this embodiment, there are four PDE4D2 CD molecules in the asymmetric unit. In this PDE4D2 crystalline form, the unit cell has dimensions of a=99.2 Å, b=111.2 Å, c=159.7 Å, and α=β=γ=90°. This crystal form can be produced in various ratios of the protein-ligand solutions versus the same well buffer, such as 1 μl to 1 μl.

The native and derivative co-crystals comprising a PDE4D2 CD and a ligand disclosed in the presently disclosed subject matter can be obtained by a variety of techniques, including batch, liquid bridge, dialysis, vapor diffusion, and hanging drop methods (see i.e., McPherson, Preparation and Analysis of Protein Crystals, John Wiley, New York, 1982; McPherson, Eur J Biochem 189:1-23, 1990; Weber, Adv Protein Chem 41:1-36, 1991). In representative embodiments, the vapor diffusion and hanging drop methods are used for the crystallization of PDE4D2 polypeptides and fragments thereof.

Native crystals of the presently disclosed subject matter can be grown by dissolving a substantially pure PDE4D2 polypeptide or a fragment thereof, and optionally a ligand, in an aqueous buffer containing a precipitant at a concentration just below that necessary to precipitate the protein. Water is removed by controlled evaporation to produce precipitating conditions, which are maintained until crystal growth ceases.

In one embodiment of the presently disclosed subject matter, native crystals are grown by vapor diffusion (See i.e., McPherson, Preparation and Analysis of Protein Crystals, John Wiley, New York, 1982; McPherson, Eur. J. Biochem 189:1-23, 1990). In this method, the polypeptide/precipitant solution is allowed to equilibrate in a closed container with a larger aqueous reservoir having a precipitant concentration optimal for producing crystals. Generally, less than about 25 μL of PDE4D2 polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand until crystals grow. Crystals generally form within two to seven days, and are thereafter suitable for data collection. Of course, those of skill in the art will recognize that the above-described crystallization procedures and conditions can be varied.

The presently disclosed subject matter also provides methods for generating a crystalline form comprising a phosphodiesterase 4D2 (PDE4D2) catalytic domain polypeptide. In one embodiment, the method comprises crystallizing the PDE4D2 catalytic domain polypeptide by vapor diffusion, whereby a crystalline form of a PDE4D2 catalytic domain polypeptide is generated. In one embodiment, the solution comprises 10-15 mg/mL PDE4D2 in a storage buffer of 50 mM NaCl, 20 mM Tris-HCl (pH 7.5), and 1 mM β-mercaptoethanol. In one embodiment, the crystalline form is grown by vapor diffusion against a well buffer comprising 100 mM HEPES (pH 7.5), 16% PEG3350, 25% ethylene glycol, 10% methanol, and 10% DMSO. In one embodiment, the crystalline form is grown at 4° C. (This is the crystallization condition for the unligated form of PDE4D2)

The presently disclosed subject matter also provides methods for generating a crystalline form comprising a phosphodiesterase 4D2 (PDE4D2) catalytic domain polypeptide in complex with a ligand. In one embodiment, the method comprises (a) incubating a solution comprising a phosphodiesterase 4D2 (PDE4D2) catalytic domain and a ligand; and (b) crystallizing the phosphodiesterase 4D2 (PDE4D2) catalytic domain polypeptide and ligand by vapor diffusion, whereby a crystalline form of a phosphodiesterase 4D2 (PDE4D2) catalytic domain polypeptide in complex with a ligand is generated. In one embodiment, the solution comprises 10 mM cAMP, 0.4 mM zinc sulfate, 15 mg/mL PDE4D2 in a storage buffer of 50 mM NaCl, 20 mM Tris-HCl (pH 7.5), and 1 mM β-mercaptoethanol. In one embodiment, the crystalline form is grown by vapor diffusion against a well buffer comprising 50 mM HEPES (pH 7.5), 15% PEG3350, 25% ethylene glycol, 5% methanol, and 5% DMSO. In one embodiment, the crystalline form is grown at 4° C.

This method is applicable to various ligands of PDE4D2 including, but not limited to cAMP. In certain embodiments, it is advantageous to saturate the PDE4D2 binding sites with ligand. In one embodiment, the method further comprises saturating cAMP binding by soaking the crystalline form in a buffer of 50 mM HEPES (pH 7.5), 20% PEG3350, 25% ethylene glycol, 0.4 mM zinc sulfate, and 50 mM cAMP. This saturation step can be performed under various conditions. In one embodiment, the saturating occurs at room temperature.

The presently disclosed subject matter also provides for a crystalline form produced by the methods.

V. Solving a Crystal Structure of the Presently Disclosed Subject Matter

Crystal structures of the presently disclosed subject matter can be solved using a variety of techniques including, but not limited to isomorphous replacement, anomalous scattering, or molecular replacement methods. Computer software packages can also be used to solve a crystal structure of the presently disclosed subject matter. Applicable software packages include, but are not limited to X-PLOR™ program (Brünger, 1992; available from Accelrys Inc, San Diego, Calif., United States of America), Xtal View (McRee, J Mol Graphics 10: 44-47, 1992; available from the San Diego Supercomputer Center, San Diego, Calif., United States of America); SHELXS 97 (Sheldrick, Acta Cryst, A46: 467, 1990; available from the Institute of Inorganic Chemistry, Georg-August-Universität, Göttingen, Germany); SOLVE (Terwilliger, T. C. and J. Berendzen. Acta Crystallographica D55:849-861, 1999; see www.solve.lanl.gov) and SHAKE-AND-BAKE (Hauptman, Curr Opin Struct Biol 7: 672-80, 1997; Weeks et al., Acta Cryst D49: 179, 1993; available from the Hauptman-Woodward Medical Research Institute, Buffalo, N.Y., United States of America). See also, Ducruix & Geige, Crystallization of Nucleic Acids and Proteins: A Practical Approach, IRL Press, Oxford, England, 1992, and references cited therein.

In one embodiment, the structure of PDE4D2 in complex with AMP is solved by the direct application of the tetramer of the PDE4D2-rolipram structure to the crystal system (Huai et al., 2003). The orientation of the individual subunits in the PDE4D2-AMP tetramer is optimized by rigid-body refinement of the Crystallography and NMR System (CNS; Brünger, 1998; see http://cns.csb.yale.edu/v1.0/). The electron density map is improved by the density modification package of CCP4 (1994). The atomic model is rebuilt by program O (Jones et al., 1991) and refined by CNS. See Table 3 for a summary of the statistics of the structure solved in this embodiment. In one embodiment, the three-dimensional structure of the crystallized complex can be determined to a resolution of about 2.3 Å or better.

VI. Overall Structure of PDE4D2 Complex

In one embodiment, the presently disclosed subject matter provides a binding site in a human PDE4D2 catalytic domain polypeptide for a substrate, wherein the substrate is in van der Waals, hydrogen bonding, or both van der Waals and hydrogen bonding contact with at least one of the following residues of the human PDE4D2 polypeptide: Tyr159, His160, His164, His200, Asp201, Met273, Asp318, Leu319, Asn321, Thr333, Ile336, Phe340, Gln369, and Phe372. In one embodiment, the binding site comprises four PDE4D2 catalytic domain polypeptides. In another embodiment, at least two of the four PDE4D2 catalytic domain polypeptides are in van der Waals, hydrogen bonding, or both van der Waal and hydrogen bonding contact through at least one of the following residues: Arg116, Met147, Thr148, Asp151, Asn214, Thr215, Asn216, Glu218, Ala220, Leu221, Met222, Tyr223, Asn224, Asp225, Asn231, Leu234, Ala235, Lys239, Gln242, Glu243, Glu244, Lys254, Arg257, Gln258, Arg261, Ile265, Arg346, Glu349, and Arg350.

VI.A. The Tetrameric Structure of the PDE4D2 Catalytic Domain

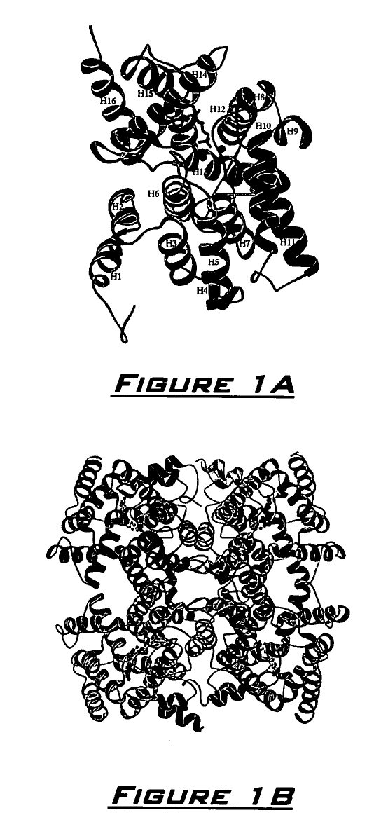

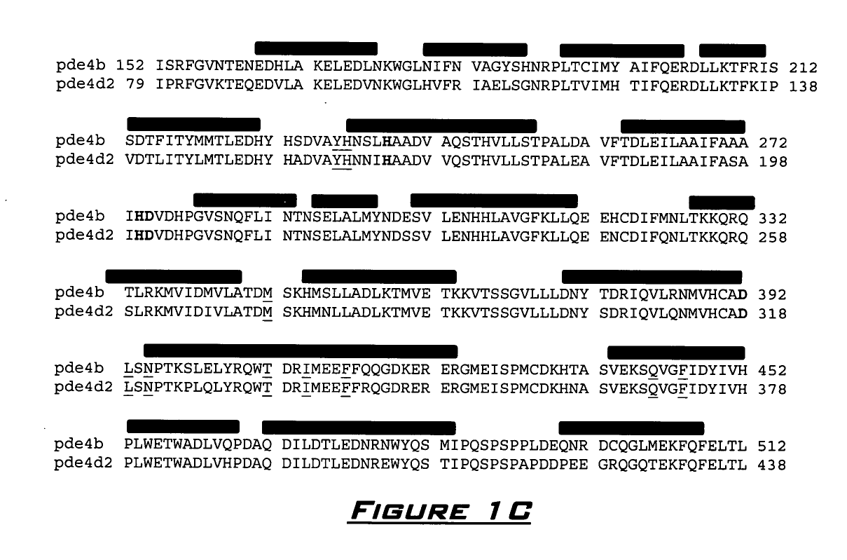

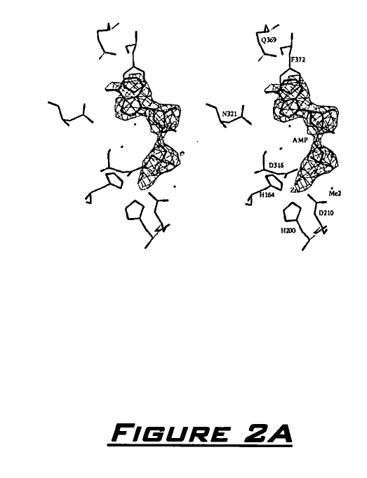

The monomer of the catalytic domain of PDE4D2 with amino acids 79-438 complexed with AMP contains sixteen alpha helices and possesses the same folding as that of PDE4B (Xu et al., 2000). In one embodiment, four molecules of the PDE4D2 catalytic domains are tightly associated into a tetramer in the crystal (FIG. 1), in comparison to a monomeric form in the PDE4B crystal. The electron density was excellent for the most amino acids of PDE4D2, except that residues 412-438 were not traceable and presumably existed in a random conformation. In contrast, residues 496-508 of PDE4B that correspond to residues 422-434 of PDE4D2 showed a helix conformation. The different oligomerization status and the conformational differences at the C-terminus between PDE4D and PDE4B could imply potential variations on the regulation of the catalysis by the PDE4 subfamilies.

The formation of the PDE4D2 tetramer is dominated by hydrogen bonds (Table 1, FIG. 1). Helices H8 to H11 form the interfaces between subunits A and B or C and D. Helices H3, H8, H10, and H14 interact with one another to form the interfaces between subunits A and C or B and D. Subunit A also crossly contacts with subunit D via helix H11, so does subunit B with C (Table 1). The C-terminal residues after 412 of PDE4D2 were not traceable in the electron density of the crystals. However, the position of helix H16 (residues 392 to 410) would predict that the C-terminus points outside of the tetrameric body, implying their potential roles in the regulation of the catalysis, instead of dimerization. This view is different from the early thought of a dimerization role of the C-terminus of PDE4 (Mehats et al., 2002). An indirect support to the regulation role of the C-terminus comes from the observation that the C-terminal helix 496-508 interacts with the active site via the crystallographic symmetry in the PDE4B structure (Xu et al., 2000).

The superposition of the PDE4D2 subunits in the tetramer shows an average RMS deviation of 0.59 Å for the backbone atoms of the four subunits, indicating the overall structural similarity among the subunits. However, significant variations on local conformations are observed for certain loops in the PDE4D tetramer. A migration up to 3.4 Å, about 5 times the average, was observed for the backbone atoms of loop Val292-Leu298. Since this loop is located far away from the active site of the enzyme, the conformational change of the loop might impact indirectly, if any, in the catalysis. In addition, the N-terminal residues 79-86 showed different conformations between subunits A and D, as revealed by their electron density. In contrast, no observable electron density for residues 79-86 of subunits B and C implies their random conformation. Finally, the averaged B-factors of 58.3 and 55.2 Å² for subunits B and C are significantly higher than 45.9 and 42.2 Å² for subunits A and D, indicating the relative conformation flexibility of subunits B and C. While it is not desired to be bound by any particular theory of operation, the interpretation to the conformational variations in the PDE4D2 tetramer could imply an allosteric regulation of the PDE4 catalysis.

VI.B. AMP Binding

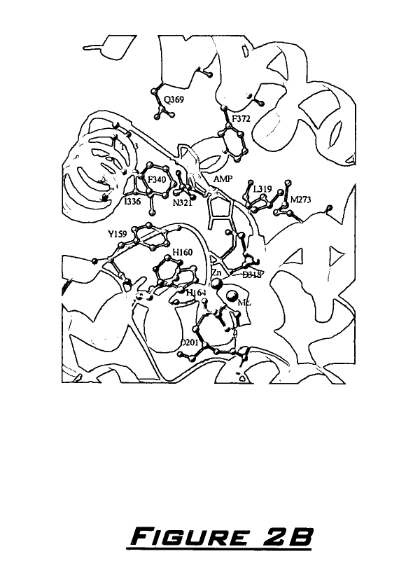

Referring now to FIG. 2, the electron density revealed occupation of the reaction product 5′-AMP in the active site of PDE4D2 in spite of that cAMP was used in the crystallization. The phosphate group of AMP directly interacts with both metal ions and forms the hydrogen bonds with His160, Asp201, and Asp318 (Table 2). It is also in a distance range of 3.2-4.0 Å to residues Tyr159, His164, and His200. The adenosine group of AMP takes an anti conformation and orients to the hydrophobic pocket made up of residues Tyr159, Leu319, Asn321, Thr333, Ile336, Gln369, and Phe372. It forms three hydrogen bonds with Gln369 and Asn321 and stacks against Phe372. The ribose of AMP has a configuration of C3′ endo puckering and makes van der Waals' contacts with PDE residues His160, Met273, Asp318, Leu319, Ile336, Phe340, and Phe372.

Mutations of the residues His160, His164, His200, Thr333, Ile336, Phe340, and Phe372 in the PDE4 subfamilies reduced or even abolished the catalytic activity (Jin et al., 1992; Pillai et al., 1993; Jacobitz et al., 1997; Atienza et al., 1999; Richter et al., 2001; Dym et al. 2002). It is also interesting to note that mutations on the corresponding AMP binding residues in other PDE families dramatically reduced the catalytic activity. For example, the mutations on the PDE3A residues Tyr751 (Tyr159 in PDE4D2), Asp950 (Asp318), Phe972 (Phe340), and Phe1004 (Phe372) made 15-280 fold loss of the catalytic efficiency (Zhang et al., 2001). The mutation of Glu672 in bovine PDE5A (Glu230 in PDE4D2), an absolutely conserved residue across the PDE families, showed a significant reduction on Kcat (Turko et al., 1998).

VI.C. Metal Binding

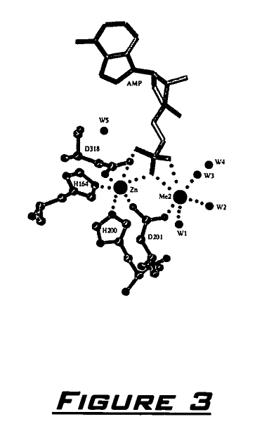

Two metal ions have been allocated to the active site of PDE4D2. The (2Fo-Fc) map revealed two strongest peaks: ˜10σ for the first metal site and ˜6σ for the second site that separate by about 3.8 Å away. Each metal ion forms six coordinations with protein residues or water molecules in a distorted octahedral configuration. As indicated in FIG. 3, the first metal coordinates with His164, His200, Asp201, Asp318, and two phosphate oxygen atoms of AMP. The second metal coordinates with Asp318, two phosphate oxygen atoms of AMP, and three bound water molecules.

The anomalous scattering experiments at the wavelength of the zinc absorption edge showed a jump of absorption, suggesting existence of zinc ion in the crystals. The first metal site has been assigned as zinc for its tight association with four protein residues and two oxygen atoms of AMP. The assignment for the second metal is difficult because of its loose binding. Zinc was used as the second metal in the structure refinement because the crystallization buffer contained 0.4 mM zinc sulfate. However, the physiological metal for the catalysis is not clear. It could be magnesium or other divalent ions as suggested by biochemical study that zinc at 1 μM concentration and other divalent metals such as Mg²⁺, Mn²⁺, Co²⁺, and Ni²⁺ at 1-10 mM concentration activate the catalysis by PDE (Hardman et al., 1971; Francis et al., 1994; Percival et al., 1997).

The first metal site was proposed to play both structural and catalytic roles because it conjoins the residues from the three subdomains of PDE4 and constitutes a physical component of the active site (Xu et al., 2000). Indeed, the structure of PDE4D2-AMP revealed the first metal ion forms two hydrogen bonds with the phosphate group, thus confirming its catalytic role (FIG. 3). The observation that both metals coordinate with the phosphate group of AMP suggests a binuclear mechanism in which the hydrolysis of cAMP/cGMP is jointly accomplished by two divalent metals. The binuclear catalysis in PDE is similar to the hydrolysis of phosphoester bonds by protein phosphatases such as calcineurin (Lohse et al., 1995; Huai et al., 2002).

The identification of the zinc ion in the crystal structure of PDE4D2-AMP is supported by the high degree of homology between two conserved HX₃HX₂₄₋₂₆E sequences of PDE and the zinc enzymes (Vallee and Auld, 1990). However, two HX₃HX₂₄₋₂₆E motifs jointly form a single pocket for binding of both metal ions in the crystal structures of PDE4B (Xu et al., 2000) and PDE4D2, instead of that each motif binds an individual metal ion as one would predict. On the other hand, the number of zinc atoms at the active sites in the different PDE families is controversial: one Zn²⁺ site per PDE4A monomer (Percival et al., 1997), two Zn²⁺ for V. Fischeri PDE (Callahan et al., 1995) and for PDE4A (Omburo et al., 1998), and three Zn²⁺ for PDE5 (Francis et al., 1994). The crystal structures of PDE4D2 and PDE4B and the structure-based sequence alignment showed the absolute conservation of the metal binding residues across the PDE families and unlikely existence of other pockets for additional metal binding. While it is not desired to be bound by a particular theory of operation, this suggests that the binuclear catalysis is a potential universal mechanism for all families of PDEs.

VI.D. Complexes of PDE4D2 with a Ligand

The presently disclosed subject matter also provides complexes of PDE4D2 with a ligand. In one embodiment, the presently disclosed subject matter provides a complex of a human PDE4D2 catalytic domain polypeptide and a substrate, wherein the substrate is in van der Waals, hydrogen bonding, or both van der Waals and hydrogen bonding contact with at least one of the following residues of the human phosphodiesterase 4D2 (PDE4D2) polypeptide: Tyr159, His160, His164, His200, Asp201, Met273, Asp318, Leu319, Asn321, Thr333, Ile336, Phe340, Gln369, and Phe372. In another embodiment, the complex comprises four PDE4D2 catalytic domain polypeptides. In another embodiment, at least two of the four PDE4D2 catalytic domain polypeptides are in van der Waals, hydrogen bonding, or both van der Waal and hydrogen bonding contact through one or more of the following residues: Arg116, Met147, Thr148, Asp151, Asn214, Thr215, Asn216, Glu218, Ala220, Leu221, Met222, Tyr223, Asn224, Asp225, Asn231, Leu234, Ala235, Lys239, Gln242, Glu243, Glu244, Lys254, Arg257, Gln258, Arg261, Ile265, Arg346, Glu349, and Arg350. In still another embodiment, the complex comprises a metal ion.

The presently disclosed subject matter also provides a crystal of the complex. In one embodiment, the crystal has the following physical measurements: space group P2₁2₁2₁; and unit cell a=99.2 Å; b=111.2 Å; c=159.7 Å.

VI.E. A Putative Mechanism of the Catalysis

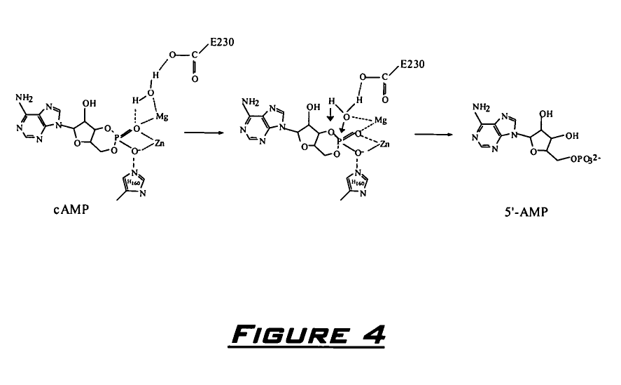

The essence of hydrolysis of a phophoester bond contains a step of nucleophilic attack by a water molecule or a hydroxide ion. The structural study of PDE4B suggested that a water molecule bridging two metals could be a candidate for the nucleophilic attack on the phosphorus atom (Xu et al. 2000). However, this water molecule is displaced by the phosphate oxygen in the PDE4D2-AMP structure and is thus unlikely to play a role in the catalysis. The crystal structure of PDE4D2-AMP showed three water molecules that form hydrogen bonds with protein residues and phosphate group of AMP and can be the potential candidates for the catalysis. Water molecule W3 (FIG. 3) is coordinated with the second metal ion and forms three hydrogen bonds respectively with side chain atom O^{e2} of Glu230, carbonyl oxygen of Thr271, and a phosphate oxygen of AMP. Water molecule W4 forms hydrogen bonds with carbonyl oxygen of Asp318, side chain atom O^{h} of Tyr159, and a phosphate oxygen of AMP. Water molecule W5 forms hydrogen bonds with side chain atom N^{e} of His204 and a phosphate oxygen of AMP. While not wishing to be bound by any particular theory of operation, W4 and W5 might play roles in orientation of the phosphate group and stabilization of the leaving group, and water W3 is the most likely candidate to serve as a nucleophile to attack the phosphoester bond. Thus, the phosphate group of cAMP at the ground state forms hydrogen bonds with His160 and the two metal ions. These hydrogen bonds can polarize the phosphodiester bond and make the phosphor atom partially positively charged. Water molecule W3, after being activated by the metal ion and Glu230, attacks the phosphorus atom, while His160 serves as a proton donor to O³, for the completion of the phosphodiester bond hydrolysis (FIG. 4).

VII. Rational Drug Design

VII.A. Generally

Modulators to polypeptides of the presently disclosed subject matter and other structurally related molecules, and complexes containing the same, may be identified and developed as set forth below and otherwise using techniques and methods known to those of skill in the art.

The presently disclosed subject matter contemplates making any molecule that is shown to modulate the activity of a polypeptide of the presently disclosed subject matter.

In another embodiment, inhibitors, modulators of the subject polypeptides, or biological complexes containing them, can be used in the manufacture of a medicament for any number of uses, including, for example, treating any disease or other treatable condition of a patient (including humans and animals), and particularly a disease caused by aberrant PDE regulation or activity.

VII.A.1 Drug Design

A number of techniques can be used to screen, identify, select, and design chemical entities capable of associating with polypeptides of the presently disclosed subject matter, structurally homologous molecules, and other molecules. Knowledge of the structure for a polypeptide of the presently disclosed subject matter, determined in accordance with the methods described herein, permits the design and/or identification of molecules and/or other modulators which have a shape complementary to the conformation of a polypeptide of the presently disclosed subject matter, or more particularly, a druggable region thereof. It is understood that such techniques and methods may use, in addition to the exact structural coordinates and other information for a polypeptide of the presently disclosed subject matter, structural equivalents thereof described above (including, for example, those structural coordinates that are derived from the structural coordinates of amino acids contained in a druggable region as described above).

The term “chemical entity”, as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (i.e., flat aromatic rings(s), puckered aliphatic rings(s), straight and branched chain aliphatics with single, double, or triple bonds) and diverse functional groups (i.e., carboxylic acids, esters, ethers, amines, aldehydes, ketones, and various heterocyclic rings).

In one aspect, the method of drug design generally includes computationally evaluating the potential of a selected chemical entity to associate with any of the molecules or complexes of the presently disclosed subject matter (or portions thereof). For example, this method may include the steps of (a) employing computational means to perform a fitting operation between the selected chemical entity and a druggable region of the molecule or complex; and (b) analyzing the results of said fitting operation to quantify the association between the chemical entity and the druggable region.

A chemical entity may be examined either through visual inspection or through the use of computer modeling using a docking program such as GRAM, DOCK, or AUTODOCK (Dunbrack et al., Folding & Design, 2: 27-42 (1997)). This procedure can include computer fitting of chemical entities to a target to ascertain how well the shape and the chemical structure of each chemical entity will complement or interfere with the structure of the subject polypeptide (Bugg et al., Scientific American, December 1993: 92-98; West et al., TIPS, 16:67-74 (1995)). Computer programs may also be employed to estimate the attraction, repulsion, and steric hindrance of the chemical entity to a druggable region, for example. Generally, the tighter the fit (i.e., the lower the steric hindrance, and/or the greater the attractive force) the more potent the chemical entity will be because these properties are consistent with a tighter binding constant. Furthermore, the more specificity in the design of a chemical entity the more likely that the chemical entity will not interfere with related proteins, which may minimize potential side-effects due to unwanted interactions.

A variety of computational methods for molecular design, in which the steric and electronic properties of druggable regions are used to guide the design of chemical entities, are known: see e.g., Cohen et al., 1990, J. Med. Chem. 33: 883-894; Kuntz et al., 1982, J. Mol. Biol. 161: 269-288; DesJarlais 1988, J. Med. Chem. 31: 722-729; Bartlett et al., 1989, Spec. Publ., Roy. Soc. Chem. 78: 182-196; Goodford et al., 1985, J. Med. Chem. 28: 849-857; DesJarlais et al., 1986, J. Med. Chem. 29: 2149-2153. Directed methods generally fall into two categories: (1) design by analogy in which 3-D structures of known chemical entities (such as from a crystallographic database) are docked to the druggable region and scored for goodness-of-fit; and (2) de novo design, in which the chemical entity is constructed piece-wise in the druggable region. The chemical entity may be screened as part of a library or a database of molecules. Databases which can be used include ACD (MDL Systems Inc., San Leandro, Calif., United States of America), NCI (National Cancer Institute, Bethesda, Md., United States of America), CCDC (Cambridge Crystallographic Data Center, Cambridge, England, United Kingdom), CAST (Chemical Abstract Service), Derwent (Derwent Information Limited, London, England, United Kingdom), Maybridge (Maybridge Chemical Company Ltd., Cornwall, England, United Kingdom), Aldrich (Aldrich Chemical Company, St. Louis, Mo., United States of America), DOCK (University of California in San Francisco, San Francisco, Calif., United States of America), and the Directory of Natural Products (Chapman & Hall). Computer programs such as CONCORD (Tripos Inc., St. Louis, Mo., United States of America) or DB-Converter (Molecular Simulations Limited, Cambridge, England, United Kingdom) can be used to convert a data set represented in two dimensions to one represented in three dimensions.

Chemical entities may be tested for their capacity to fit spatially with a druggable region or other portion of a target protein. As used herein, the term “fits spatially” means that the three-dimensional structure of the chemical entity is accommodated geometrically by a druggable region. A favorable geometric fit occurs when the surface area of the chemical entity is in close proximity with the surface area of the druggable region without forming unfavorable interactions. A favorable complementary interaction occurs where the chemical entity interacts by hydrophobic, aromatic, ionic, dipolar, or hydrogen donating and accepting forces. Unfavorable interactions may be steric hindrance between atoms in the chemical entity and atoms in the druggable region.

If a model of the presently disclosed subject matter is a computer model, the chemical entities may be positioned in a druggable region through computational docking. If, on the other hand, the model of the presently disclosed subject matter is a structural model, the chemical entities may be positioned in the druggable region by, for example, manual docking. As used herein the term “docking” refers to a process of placing a chemical entity in close proximity with a druggable region, or a process of finding low energy conformations of a chemical entity/druggable region complex.

In an illustrative embodiment, the design of potential modulator begins from the general perspective of shape complimentary for the druggable region of a polypeptide of the presently disclosed subject matter, and a search algorithm is employed which is capable of scanning a database of small molecules of known three-dimensional structure for chemical entities which fit geometrically with the target druggable region. Most algorithms of this type provide a method for finding a wide assortment of chemical entities that are complementary to the shape of a druggable region of the subject polypeptide. Each of a set of chemical entities from a particular data-base, such as the Cambridge Crystallographic Data Bank (CCDB) (Allen et al., 1973, J. Chem. Doc. 13: 119), is individually docked to the druggable region of a polypeptide of the presently disclosed subject matter in a number of geometrically permissible orientations with use of a docking algorithm. In certain embodiments, a set of computer algorithms called DOCK, can be used to characterize the shape of invaginations and grooves that form the active sites and recognition surfaces of the druggable region (Kuntz et al., 1982, J. Mol. Biol. 161: 269-288). The program can also search a database of small molecules for templates whose shapes are complementary to particular binding sites of a polypeptide of the presently disclosed subject matter (DesJarlais et al., 1988, J Med Chem 31: 722-729).

The orientations are evaluated for goodness-of-fit and the best are kept for further examination using molecular mechanics programs, such as AMBER or CHARMM. Such algorithms have previously proven successful in finding a variety of chemical entities that are complementary in shape to a druggable region.

Goodford (1985, J Med Chem 28:849-857) and Boobbyer et al. (1989, J Med Chem 32:1083-1094) have produced a computer program (GRID) that seeks to determine regions of high affinity for different chemical groups (termed probes) of the druggable region. GRID hence provides a tool for suggesting modifications to known chemical entities that might enhance binding. It may be anticipated that some of the sites discerned by GRID as regions of high affinity correspond to “pharmacophoric patterns” determined inferentially from a series of known ligands. As used herein, a “pharmacophoric pattern” is a geometric arrangement of features of chemical entities that is believed to be important for binding. Attempts have been made to use pharmacophoric patterns as a search screen for novel ligands (Jakes et al., 1987 J Mol Graph 5:41-48; Brint et al., 1987, J Mol Graph 5:49-56; Jakes et al., 1986, J Mol Graph 4:12-20).

Yet a further embodiment of the presently disclosed subject matter utilizes a computer algorithm such as CLIX which searches such databases as CCDB for chemical entities which can be oriented with the druggable region in a way that is both sterically acceptable and has a high likelihood of achieving favorable chemical interactions between the chemical entity and the surrounding amino acid residues. The method is based on characterizing the region in terms of an ensemble of favorable binding positions for different chemical groups and then searching for orientations of the chemical entities that cause maximum spatial coincidence of individual candidate chemical groups with members of the ensemble. The algorithmic details of CLIX is described in Lawrence et al., 1992, Proteins 12:31-41.

In this way, the efficiency with which a chemical entity may bind to or interfere with a druggable region may be tested and optimized by computational evaluation. For example, for a favorable association with a druggable region, a chemical entity must preferably demonstrate a relatively small difference in energy between its bound and fine states (i.e., a small deformation energy of binding). Thus, certain, more desirable chemical entities will be designed with a deformation energy of binding of not greater than about 10 kcal/mole, and more preferably, not greater than 7 kcal/mole. Chemical entities may interact with a druggable region in more than one conformation that is similar in overall binding energy. In those cases, the deformation energy of binding is taken to be the difference between the energy of the free entity and the average energy of the conformations observed when the chemical entity binds to the target.

In this way, the presently disclosed subject matter provides computer-assisted methods for identifying or designing a potential modulator of the activity of a polypeptide of the presently disclosed subject matter including: supplying a computer modeling application with a set of structure coordinates of a molecule or complex, the molecule or complex including at least a portion of a druggable region from a polypeptide of the presently disclosed subject matter; supplying the computer modeling application with a set of structure coordinates of a chemical entity; and determining whether the chemical entity is expected to bind to the molecule or complex, wherein binding to the molecule or complex is indicative of potential modulation of the activity of a polypeptide of the presently disclosed subject matter.

In another aspect, the presently disclosed subject matter provides a computer-assisted method for identifying or designing a potential modulator to a polypeptide of the presently disclosed subject matter, supplying a computer modeling application with a set of structure coordinates of a molecule or complex, the molecule or complex including at least a portion of a druggable region of a polypeptide of the presently disclosed subject matter; supplying the computer modeling application with a set of structure coordinates for a chemical entity; evaluating the potential binding interactions between the chemical entity and active site of the molecule or molecular complex; structurally modifying the chemical entity to yield a set of structure coordinates for a modified chemical entity, and determining whether the modified chemical entity is expected to bind to the molecule or complex, wherein binding to the molecule or complex is indicative of potential modulation of the polypeptide of the presently disclosed subject matter.

In one embodiment, a potential modulator can be obtained by screening a peptide library (Scott & Smith, 1990, Science, 249: 386-390; Cwirla et al., 1990, Proc. Natl. Acad. Sci. USA, 87: 6378-6382; Devlin et al., 1990, Science, 249: 404-406). A potential modulator selected in this manner could then be systematically modified by computer modeling programs until one or more promising potential drugs are identified. Such analysis has been shown to be effective in the development of HIV protease inhibitors (Lam et al., 1994, Science 263: 380-384; Wlodawer et al., 1993, Ann. Rev. Biochem. 62: 543-585; Appelt, 1993, Perspectives in Drug Discovery and Design 1: 23-48; Erickson, 1993, Perspectives in Drug Discovery and Design 1: 109-128). Alternatively a potential modulator may be selected from a library of chemicals such as those that can be licensed from third parties, such as chemical and pharmaceutical companies. A third alternative is to synthesize the potential modulator de novo.

For example, in certain embodiments, the presently disclosed subject matter provides a method for making a potential modulator for a polypeptide of the presently disclosed subject matter, the method including synthesizing a chemical entity or a molecule containing the chemical entity to yield a potential modulator of a polypeptide of the presently disclosed subject matter, the chemical entity having been identified during a computer-assisted process including supplying a computer modeling application with a set of structure coordinates of a molecule or complex, the molecule or complex including at least one druggable region from a polypeptide of the presently disclosed subject matter; supplying the computer modeling application with a set of structure coordinates of a chemical entity; and determining whether the chemical entity is expected to bind to the molecule or complex at the active site, wherein binding to the molecule or complex is indicative of potential modulation. This method may further include the steps of evaluating the potential binding interactions between the chemical entity and the active site of the molecule or molecular complex and structurally modifying the chemical entity to yield a set of structure coordinates for a modified chemical entity, which steps may be repeated one or more times.

Once a potential modulator is identified, it can then be tested in any standard assay for the macromolecule depending of course on the macromolecule, including in high throughput assays. Further refinements to the structure of the modulator will generally be necessary and can be made by the successive iterations of any and/or all of the steps provided by the particular screening assay, in particular further structural analysis by i.e., 15N NMR relaxation rate determinations or x-ray crystallography with the modulator bound to the subject polypeptide. These studies may be performed in conjunction with biochemical assays.

Once identified, a potential modulator may be used as a model structure, and analogs to the compound can be obtained. The analogs are then screened for their ability to bind the subject polypeptide. An analog of the potential modulator might be chosen as a modulator when it binds to the subject polypeptide with a higher binding affinity than the predecessor modulator.

In a related approach, iterative drug design is used to identify modulators of a target protein. Iterative drug design is a method for optimizing associations between a protein and a modulator by determining and evaluating the three dimensional structures of successive sets of protein/modulator complexes. In iterative drug design, crystals of a series of protein/modulator complexes are obtained and then the three-dimensional structures of each complex is solved. Such an approach provides insight into the association between the proteins and modulators of each complex. For example, this approach may be accomplished by selecting modulators with inhibitory activity, obtaining crystals of this new protein/modulator complex, solving the three dimensional structure of the complex, and comparing the associations between the new protein/modulator complex and previously solved protein/modulator complexes. By observing how changes in the modulator affected the protein/modulator associations, these associations may be optimized.

In addition to designing and/or identifying a chemical entity to associate with a druggable region, as described above, the same techniques and methods may be used to design and/or identify chemical entities that either associate, or do not associate, with affinity regions, selectivity regions or undesired regions of protein targets. By such methods, selectivity for one or a few targets, or alternatively for multiple targets, from the same species or from multiple species, can be achieved.

For example, a chemical entity may be designed and/or identified for which the binding energy for one druggable region, i.e., an affinity region or selectivity region, is more favorable than that for another region, i.e., an undesired region, by about 20%, 30%, 50% to about 60% or more. It may be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison may be made by reference to the K_{d}, usually the apparent K_{d}, of said chemical entity with the two or more regions in question.

In another aspect, prospective modulators are screened for binding to two nearby druggable regions on a target protein. For example, a modulator that binds a first region of a target polypeptide does not bind a second nearby region. Binding to the second region can be determined by monitoring changes in a different set of amide chemical shifts in either the original screen or a second screen conducted in the presence of a modulator (or potential modulator) for the first region. From an analysis of the chemical shift changes, the approximate location of a potential modulator for the second region is identified. Optimization of the second modulator for binding to the region is then carried out by screening structurally related compounds (i.e., analogs as described above). When modulators for the first region and the second region are identified, their location and orientation in the ternary complex can be determined experimentally. On the basis of this structural information, a linked compound, i.e., a consolidated modulator, is synthesized in which the modulator for the first region and the modulator for the second region are linked. In certain embodiments, the two modulators are covalently linked to form a consolidated modulator. This consolidated modulator may be tested to determine if it has a higher binding affinity for the target than either of the two individual modulators. A consolidated modulator is selected as a modulator when it has a higher binding affinity for the target than either of the two modulators. Larger consolidated modulators can be constructed in an analogous manner, i.e., linking three modulators which bind to three nearby regions on the target to form a multilinked consolidated modulator that has an even higher affinity for the target than the linked modulator. In this example, it is assumed that is desirable to have the modulator bind to all the druggable regions. However, it may be the case that binding to certain of the druggable regions is not desirable, so that the same techniques may be used to identify modulators and consolidated modulators that show increased specificity based on binding to at least one but not all druggable regions of a target.

The presently disclosed subject matter provides a number of methods that use drug design as described above. For example, in one aspect, the presently disclosed subject matter contemplates a method for designing a candidate compound for screening for inhibitors of a polypeptide of the presently disclosed subject matter, the method comprising: (a) determining the three dimensional structure of a crystallized polypeptide of the presently disclosed subject matter or a fragment thereof; and (b) designing a candidate inhibitor based on the three dimensional structure of the crystallized polypeptide or fragment.

In another aspect, the presently disclosed subject matter contemplates a method for identifying a potential inhibitor of a polypeptide of the presently disclosed subject matter, the method comprising: (a) providing the three-dimensional coordinates of a polypeptide of the presently disclosed subject matter or a fragment thereof; (b) identifying a druggable region of the polypeptide or fragment; and (c) selecting from a database at least one compound that comprises three dimensional coordinates which indicate that the compound may bind the druggable region; (d) wherein the selected compound is a potential inhibitor of a polypeptide of the presently disclosed subject matter.

In another aspect, the presently disclosed subject matter contemplates a method for identifying a potential modulator of a molecule comprising a druggable region similar to that of SEQ ID NO: 2 or SEQ ID NO: 4, the method comprising: (a) using the atomic coordinates of amino acid residues from SEQ ID NO: 2 or SEQ ID NO: 4, or a fragment thereof, ±a root mean square deviation from the backbone atoms of the amino acids of not more than 1.5 Å, to generate a three-dimensional structure of a molecule comprising a druggable region that is a portion of SEQ ID NO: 2 or SEQ ID NO: 4; (b) employing the three dimensional structure to design or select the potential modulator; (c) synthesizing the modulator; and (d) contacting the modulator with the molecule to determine the ability of the modulator to interact with the molecule.

In another aspect, the presently disclosed subject matter contemplates an apparatus for determining whether a compound is a potential inhibitor of a polypeptide having SEQ ID NO: 2 or SEQ ID NO: 4, the apparatus comprising: (a) a memory that comprises: (i) the three dimensional coordinates and identities of the atoms of a polypeptide of the presently disclosed subject matter or a fragment thereof that form a druggable site; and (ii) executable instructions; and (b) a processor that is capable of executing instructions to: (i) receive three-dimensional structural information for a candidate compound; (ii) determine if the three-dimensional structure of the candidate compound is complementary to the structure of the interior of the druggable site; and (iii) output the results of the determination.

In another aspect, the presently disclosed subject matter contemplates a method for designing a potential compound for the prevention or treatment of a disease or disorder, the method comprising: (a) providing the three dimensional structure of a crystallized polypeptide of the presently disclosed subject matter, or a fragment thereof; (b) synthesizing a potential compound for the prevention or treatment of a disease or disorder based on the three dimensional structure of the crystallized polypeptide or fragment; (c) contacting a polypeptide of the presently disclosed subject matter or a PDE with the potential compound; and (d) assaying the activity of a polypeptide of the presently disclosed subject matter, wherein a change in the activity of the polypeptide indicates that the compound may be useful for prevention or treatment of a disease or disorder.

In another aspect, the presently disclosed subject matter contemplates a method for designing a potential compound for the prevention or treatment of a disease or disorder, the method comprising: (a) providing structural information of a druggable region derived from NMR spectroscopy of a polypeptide of the presently disclosed subject matter, or a fragment thereof; (b) synthesizing a potential compound for the prevention or treatment of a disease or disorder based on the structural information; (c) contacting a polypeptide of the presently disclosed subject matter or a PDE with the potential compound; and (d) assaying the activity of a polypeptide of the presently disclosed subject matter, wherein a change in the activity of the polypeptide indicates that the compound may be useful for prevention or treatment of a disease or disorder.

VII.B. Methods of Designing PDE4D2 CD Ligand Compounds

The present X-ray structure of PDE4D2 bound to AMP provides an accurate three-dimensional structure of the catalytic pocket of PDE4D2. Novel ligands can be designed to fit this specific pocket using a variety of computational methods, discussed below. Alternatively, known ligands can be docked into the catalytic pocket, using a variety of docking programs and algorithms. These docked structures can be examined graphically to suggest chemical modifications that would improve their fit to the pocket, or their binding to the pocket. Alternatively, known ligands can be complexed with the PDE4D2 protein and crystallized using the methods of presently disclosed subject matter, allowing the structure of the complex to be determined by X-ray crystallography. The three dimensional structures can be examined graphically to suggest chemical modifications that would improve their fit to the pocket, or their binding to the pocket.

The present X-ray structure of PDE4D2 can also be used as a template to build a three-dimensional model of the inhibitor structure of other PDE families. Specifically, various computer software programs can be used to design novel ligands that would fit the specific pocket in the model for PDE4D2. Docking calculations can be used to predict how known PDE4D2 inhibitors will bind to the catalytic pocket of PDE4D2. These predicted complex structures can then be examined by computer graphics to suggest specific chemical modifications that would enhance the binding to the activated state of PDE4D2.

To be useful as a therapeutic agent, a chemical compound that acts through PDE4D2 must reduce PDE4D2 activity to an appropriate level in relevant tissues. In principle, this can be achieved by adjusting the PDE4D2 conformational equilibrium so that appropriate fractions of the PDE4D2 protein exist in the activated and inactivated states. This in turn can be achieved with ligands that bind almost exclusively to one or the other of the two major conformational states. The design of ligands that are selective for a specific conformational state is facilitated by consideration of how these ligands might bind to each of the two conformational states. Binding modes can be obtained using docking calculations, and then examined graphically to suggest chemical modifications that would make binding to a particular conformational state either more favorable or less favorable. Iterative application of these techniques can yield ligands with the desired level of selectivity for the particular conformational state of PDE4D2, thereby achieving the desired level of PDE4D2 activity. Ligands that can bind to both conformational states of the PDE4D2 protein can also be designed. This is also facilitated by consideration of how the ligands might bind to each of the two conformational states, using the same approach as discussed above, but this time seeking chemical structures and chemical modifications that would permit binding to both conformational states.

The methods of presently disclosed subject matter can also be used to suggest possible chemical modifications of a compound that might reduce or minimize its effect on PDE4D2. This approach may be useful in drug discovery projects aiming to find compounds that modulate the activity of some other target molecule, where modulation of PDE4D2 activity is an undesirable side effect. This approach is useful in engineering PDE4D2 activity out of other, non-drug molecules. Humans and other animals are exposed to a wide range of different chemical compounds, some of which might act on PDE4D2 in an undesirable manner. Such a compound could be complexed with PDE4D2 and crystallized using the methods of the presently disclosed subject matter. The structure could then be determined by X-ray crystallography. Alternatively, the structure of the complex could be predicted computationally using molecular docking software. In this case, compounds that tend to activate PDE4D2 would be docked into a model or structure of the activated form of PDE4D2, whereas compounds that tend to reduce the activity of PDE4D2 would be docked into a model or structure of an inactivated form of PDE4D2, such as the complex presented here.

Whether the structure is obtained by X-ray crystallography or computational methods, the structure would be examined by computer graphics to suggest chemical modifications that would minimize the tendency to bind to PDE4D2. For example, substituents could be introduced onto the compound that would project into volume occupied by the PDE4D2 protein. Alternatively, a region of the molecule that binds to a lipophilic region of the PDE4D2 binding site could be modified to make it more polar, thus reducing its tendency to bind to PDE4D2. Alternatively, a polar group of the compound that makes a hydrogen bonding interaction with PDE4D2 could be identified and modified to an alternative group that fails to make the hydrogen bond. Appropriate chemical modifications can be chosen such that the desirable properties and behavior of the compound would be retained.

The design of candidate substances, also referred to as “compounds” or “candidate compounds”, that bind to or inhibit PDE CD (for example, PDE4D2 CD)-mediated activity according to the presently disclosed subject matter generally involves consideration of two factors. First, the compound must be capable of chemically and structurally associating with a PDE CD. Non-covalent molecular interactions important in the association of a PDE CD with its substrate include hydrogen bonding, van der Waals interactions, and hydrophobic interactions. The interaction between an atom of a CD amino acid and an atom of a CD ligand can be made by any force or attraction described in nature. Usually the interaction between the atom of the amino acid and the ligand will be the result of a hydrogen bonding interaction, charge interaction, hydrophobic interaction, van der Waals interaction, or dipole interaction. In the case of the hydrophobic interaction, it is recognized that this is not a per se interaction between the amino acid and ligand, but rather the usual result, in part, of the repulsion of water or other hydrophilic group from a hydrophobic surface. Reducing or enhancing the interaction of the CD and a ligand can be measured by calculating or testing binding energies, either computationally or using thermodynamic or kinetic methods known in the art.

Second, the compound must be able to assume a conformation that allows it to associate with a PDE CD. Although certain portions of the compound will not directly participate in this association with a PDE CD, those portions can still influence the overall conformation of the molecule. This influence on conformation, in turn, can have a significant impact on potency. Such conformational requirements include the overall three-dimensional structure and orientation of the chemical entity or compound in relation to all or a portion of the binding site, i.e., the catalytic pocket or an accessory binding site of a PDE CD, or the spacing between functional groups of a compound comprising several chemical entities that directly interact with a PDE CD.

Chemical modifications can enhance or reduce interactions of an atom of a CD amino acid and an atom of an CD ligand. Steric hindrance can be a common approach for changing the interaction of a CD binding pocket with an activation domain. Chemical modifications are introduced in one embodiment at C—H, C—, and C—OH positions in a ligand, where the carbon is part of the ligand structure that remains the same after modification is complete. In the case of C—H, C could have 1, 2, or 3 hydrogens, but usually only one hydrogen will be replaced. The H or OH can be removed after modification is complete and replaced with a desired chemical moiety.

The potential binding effect of a chemical compound on a PDE4D2 catalytic domain can be analyzed prior to its actual synthesis and testing by the use of computer modeling techniques that employ the coordinates of a crystalline PDE CD, for example a PDE4D2 CD polypeptide of the presently disclosed subject matter. If the theoretical structure of the given compound suggests insufficient interaction and association between it and a PDE CD, synthesis and testing of the compound is obviated. However, if computer modeling indicates a strong interaction, the molecule can then be synthesized and tested for its ability to bind and modulate the activity of a PDE CD. In this manner, synthesis of unproductive or inoperative compounds can be avoided.

Interacting amino acids forming contacts with a ligand and the atoms of the interacting amino acids are usually 2 to 4 Å away from the center of the atoms of the ligand. Generally these distances are determined by computer as discussed herein and in McRee (McRee, Practical Protein Crystallography, Academic Press, New York, 1993). However distances can be determined manually once the three dimensional model is made. More commonly, the atoms of the ligand and the atoms of interacting amino acids are 3 to 4 Å apart. A ligand can also interact with distant amino acids, after chemical modification of the ligand to create a new ligand. Distant amino acids are generally not in contact with the ligand before chemical modification. A chemical modification can change the structure of the ligand to make as new ligand that interacts with a distant amino acid usually at least 4.5 Å away from the ligand. Distant amino acids rarely line the surface of the binding cavity for the ligand, as they are too far away from the ligand to be part of a pocket or surface of the binding cavity.

In one embodiment, the presently disclosed subject matter provides a method for designing a ligand of a PDE4D2 polypeptide, the method comprising (a) forming a complex of a compound bound to the PDE4D2 polypeptide; (b) determining a structural feature of the complex formed in (a); wherein the structural feature is of a binding site for the compound; and (c) using the structural feature determined in (b) to design a ligand of a PDE4D2 polypeptide capable of binding to the binding site of PDE4D2.

Optionally, a method for designing a ligand of a PDE4D2 polypeptide can further comprise using a computer-based model of the complex formed in (a) in designing the ligand. In one embodiment, a compound designed or selected as binding to a PDE polypeptide (in one embodiment a PDE4D2 CD polypeptide) can be further computationally optimized so that in its bound state it would lack repulsive electrostatic interaction with the target polypeptide. Such non-complementary (i.e., electrostatic) interactions include repulsive charge-charge, dipole-dipole, and charge-dipole interactions. Specifically, the sum of all electrostatic interactions between the ligand and the polypeptide when the ligand is bound to a PDE CD make a neutral or favorable contribution to the enthalpy of binding.

In another embodiment, a method for designing a ligand of a PDE4D2 polypeptide comprises (a) selecting a candidate PDE4D2 ligand; (b) determining which amino acid or amino acids of a PDE4D2 polypeptide interact with the ligand using a three-dimensional model of a crystallized protein, the model comprising a PDE4D2 catalytic domain in complex with a ligand; (c) identifying in a biological assay for PDE4D2 activity a degree to which the ligand modulates the activity of the PDE4D2 polypeptide; (d) selecting a chemical modification of the ligand wherein the interaction between the amino acids of the PDE4D2 polypeptide and the ligand is predicted to be modulated by the chemical modification; (e) synthesizing a ligand having the chemical modified to form a modified ligand; (f) contacting the modified ligand with the PDE4D2 polypeptide; (g) identifying in a biological assay for PDE4D2 activity a degree to which the modified ligand modulates the biological activity of the PDE4D2 polypeptide; and (h) comparing the biological activity of the PDE4D2 polypeptide in the presence of modified ligand with the biological activity of the PDE4D2 polypeptide in the presence of the unmodified ligand, whereby a ligand of a PDE4D2 polypeptide is designed. In one embodiment, the PDE4D2 polypeptide is a human PDE4D2 polypeptide. In another embodiment, the PDE4D2 polypeptide comprises the amino acid sequence of SEQ ID NO:2. In another embodiment, the method further comprises repeating steps (a) through (f), if the biological activity of the PDE4D2 polypeptide in the presence of the modified ligand varies from the biological activity of the PDE4D2 polypeptide in the presence of the unmodified ligand.

The presently disclosed subject matter also provides methods for identifying ligands of PDE4D2. In one embodiment, a method for identifying a PDE4D2 ligand can comprise (a) providing atomic coordinates of a phosphodiesterase 4D2 (PDE4D2) catalytic domain in complex with a ligand to a computerized modeling system; and (b) modeling a ligand that fits spatially into the binding site of the PDE4D2 catalytic domain to thereby identify a PDE4D2 ligand. In one embodiment, the PDE4D2 catalytic domain comprises the amino acid sequence of SEQ ID NO: 4. In another embodiment, the method further comprises identifying in an assay for PDE4D2-mediated activity a modeled ligand that increases or decreases the activity of the PDE4D2.

In another embodiment, the presently disclosed subject matter provides a method of identifying a PDE4D2 ligand that selectively binds a PDE4D2 polypeptide compared to other polypeptides, the method comprising: (a) providing atomic coordinates of a PDE4D2 catalytic domain in complex with a ligand to a computerized modeling system; and (b) modeling a ligand that fits into the binding pocket of a PDE4D2 catalytic domain and that interacts with residues of a PDE4D2 catalytic domain that are conserved among PDE4D2 subtypes to thereby identify a PDE4D2 ligand that selectively binds a PDE4D2 polypeptide compared to other polypeptides. In one embodiment, the PDE4D2 catalytic domain comprises the amino acid sequence shown in SEQ ID NO: 4. In another embodiment, the method further comprises identifying in a biological assay for PDE4D2 activity a modeled ligand that selectively binds to said PDE4D2 and increases or decreases the activity of the PDE4D2.

One of several methods can be used to screen chemical entities or fragments for their ability to associate with a PDE CD and, more particularly, with the individual binding sites of a PDE CD, such as a catalytic pocket or an accessory binding site. This process can begin by visual inspection of, for example, a catalytic pocket on a computer screen based on the PDE4D2 CD atomic coordinates disclosed in Tables 4 and 5. Selected fragments or chemical entities can then be positioned in a variety of orientations, or docked, within an individual binding site of a PDE4D2 CD as defined herein above. Docking can be accomplished using software programs such as those available under the trade names QUANTA™ (available from Accelrys Inc, San Diego, Calif., United States of America) and SYBYL™ (available from Tripos, Inc., St. Louis, Mo., United States of America), followed by energy minimization and molecular dynamics with standard molecular mechanics force fields, such as CHARM (Brooks et al., J Comp Chem, 8: 132, 1993) and AMBER 5 (Case et al., AMBER 5, University of California, San Francisco, 1997; Pearlman et al., Comput Phys Commun, 91: 1-41, 1995).

Specialized computer programs can also assist in the process of selecting fragments or chemical entities. These include:

1. GRID™ program, version 17 (Goodford, J Med Chem, 28: 849-57, 1985), which is available from Molecular Discovery Ltd. of Oxford, United Kingdom;

2. MCSS™ program (Miranker & Karplus, Proteins, 11:29-34, 1991), which is available from Accelrys Inc, San Diego, Calif., United States of America;

3. AUTODOCK™ 3.0 program (Goodsell & Olsen, Proteins, 8:195-202, 1990), which is available from the Scripps Research Institute, La Jolla, Calif., United States of America;

4. DOCK™ 4.0 program (Kuntz et al., J Mol Biol, 161:269-88, 1982), which is available from the University of California, San Francisco, Calif., United States of America;

5. FLEX-X™ program (See Rarey et al., J Comput Aid Mol Des, 10:41-54, 1996), which is available from Tripos, Inc. of St. Louis, Mo., United States of America;

6. MVP program (Lambert, in Practical Application of Computer-Aided Drug Design, Charifson, ed. Marcel-Dekker, New York, pp. 243-303, 1997); and

7. LUDI™ program (Bohm, J Comput Aid Mol Des, 6: 61-78, 1992), which is available from Accelrys Inc, San Diego, Calif., United States of America.

Once suitable chemical entities or fragments have been selected, they can be assembled into a single compound or ligand. Assembly can proceed by visual inspection of the relationship of the fragments to each other on the three-dimensional image displayed on a computer screen in relation to the structure coordinates of a PDE4D2 CD complex, optionally in further complex with a ligand. Manual model building using software such as QUANTA™ or SYBYL™ typically follows.

Useful programs to aid one of ordinary skill in the art in connecting the individual chemical entities or fragments include:

1. CAVEAT™ program (Bartlett et al., Special Pub., Royal Chem Soc, 78:182-96, 1989), which is available from the University of California, Berkeley, Calif., United States of America;

2. 3D Database systems, such as MACCS-3D™ system program, which is available from MDL Information Systems of San Leandro, Calif., United States of America. This area is reviewed in Martin, J Med Chem 35:2145-54, 1992; and

3. HOOK™ program (Eisen et al., Proteins, 19:199-221, 1994), which is available from Accelrys Inc, San Diego, Calif., United States of America.

Instead of proceeding to build a PDE CD polypeptide ligand (in one embodiment a PDE4D2 CD ligand) in a step-wise fashion one fragment or chemical entity at a time as described above, ligand compounds can be designed as a whole or de novo using the structural coordinates of a crystalline PDE4D2 CD polypeptide of the presently disclosed subject matter and either an empty binding site or optionally including some portion(s) of a known ligand(s). Applicable methods can employ the following software programs:

1. LUDI™ program (Bohm, J Comput Aid Mol Des, 6:61-78, 1992), which is available from Accelrys Inc, San Diego, Calif., United States of America;

2. LEGEND™ program (Nishibata & Itai, Tetrahedron, 47:8985); and

3. LEAPFROG™, which is available from Tripos Associates of St. Louis, Mo., United States of America.

Other molecular modeling techniques can also be employed in accordance with presently disclosed subject matter. See i.e., Cohen et al., J Med Chem, 33:883-94, 1990; Navia & Murcko, Curr Opin Struct Biol, 2:202-10, 1992; and U.S. Pat. No. 6,008,033 to Abdel-Meguid, et al., all of which are incorporated herein by reference.

Once a compound has been designed or selected by the above methods, the efficiency with which that compound can bind to a PDE CD can be tested and optimized by computational evaluation. By way of a particular example, a compound that has been designed or selected to function as a PDE4D2 CD ligand can traverse a volume not overlapping that occupied by the binding site when it is bound to its native ligand. Additionally, an effective PDE CD ligand can demonstrate a relatively small difference in energy between its bound and free states (i.e., a small deformation energy of binding). Thus, the most efficient PDE CD ligands can be designed with a deformation energy of binding of in one embodiment not greater than about 10 kcal/mole, and in another embodiment not greater than 7 kcal/mole. It is possible for PDE CD ligands to interact with the polypeptide in more than one conformation that is similar in overall binding energy. In those cases, the deformation energy of binding is taken to be the difference between the energy of the free compound and the average energy of the conformations observed when the ligand binds to the polypeptide.

A compound designed or selected as binding to a PDE CD polypeptide (in one embodiment a PDE4D2 polypeptide, and in another embodiment a PDE4D2 CD polypeptide) can be further computationally optimized so that in its bound state it would preferably lack repulsive electrostatic interaction with the target polypeptide. Such non-complementary (i.e., electrostatic) interactions include repulsive charge-charge, dipole-dipole, and charge-dipole interactions. Specifically, the sum of all electrostatic interactions between the ligand and the polypeptide when the ligand is bound to a PDE CD preferably make a neutral or favorable contribution to the enthalpy of binding.

Specific computer software is available in the art to evaluate compound deformation energy and electrostatic interaction. Examples of programs designed for such uses include:

1. GAUSSIAN 98™, which is available from Gaussian, Inc. of Pittsburgh, Pa., United States of America;

2. AMBER™ program, version 6.0, which is available from the University of California, San Francisco, Calif., United States of America;

3. QUANTA™ program, which is available from Accelrys Inc, San Diego, Calif., United States of America;

4. CHARMM® program, which is available from Accelrys Inc, San Diego, Calif., United States of America; and

4. INSIGHT II® program, which is available from Accelrys Inc, San Diego, Calif., United States of America.

These programs can be implemented using a suitable computer system. Other hardware systems and software packages will be apparent to those skilled in the art after review of the disclosure of the presently disclosed subject matter presented herein.

Once a PDE CD modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity, and charge as the original group. Components known in the art to alter conformation are avoided. Such substituted chemical compounds can then be analyzed for efficiency of fit to a PDE CD binding site using the same computer-based approaches described in detail above.

VII.C. Sterically Similar Compounds

A further aspect of the presently disclosed subject matter is that sterically similar compounds can be formulated to mimic the key portions of a PDE4D2 CD structure. Such compounds are functional equivalents. The generation of a structural functional equivalent can be achieved by the techniques of modeling and chemical design known to those of skill in the art and described herein. Modeling and chemical design of PDE4D2 and PDE4D2 CD structural equivalents can be based on the structure coordinates of a crystalline PDE4D2 CD polypeptide of the presently disclosed subject matter. It will be understood that all such sterically similar constructs fall within the scope of the presently disclosed subject matter.

VII.D. Designing a PDE4D2 Modulator

The presently disclosed subject matter also provides methods for designing PDE4D2 modulators. In one embodiment, a method of designing a chemical compound that modulates the biological activity of a target PDE4D2 polypeptide comprises (a) obtaining three-dimensional structures for a catalytic domain (CD) of PDE4D2 bound to a ligand, and wherein the structures are selected from the group consisting of X-ray structures and computer generated models; (b) rotating and translating the three-dimensional structures as rigid bodies so as to superimpose corresponding backbone atoms of a core region of the PDE4D2 CD; (c) comparing the superimposed three-dimensional structures to identify volume near a catalytic pocket of the PDE CD that is available to a ligand in one or more structures, but not available to the ligand in one or more other structures; (d) designing a chemical compound that could occupy the volume in some of the complexed structures, but not in others; (e) synthesizing the designed chemical compound; and (f) testing the designed chemical compound in a biological assay to determine whether it acts as a ligand of PDE4D2 with a desired effect on PDE4D2 biological activities, whereby a ligand of a PDE4D2 polypeptide is designed.

In another embodiment, the present method further comprises designing a chemical compound by considering a known agonist of the PDE CD and adding a substituent that protrudes into the volume identified in step (c) or that makes a desired interaction. For any this embodiment, the designing a chemical compound can further comprise using computer modeling software as discussed hereinabove.

In another embodiment, the presently disclosed subject matter also provides a method of designing a ligand that selectively modulates the activity of a PDE4D2 polypeptide comprising (a) evaluating a three-dimensional structure of a crystallized PDE4D2 catalytic domain polypeptide in complex with a ligand; and (b) synthesizing a potential ligand based on the three-dimensional structure of the crystallized PDE4D2 catalytic polypeptide in complex with a ligand, whereby a ligand that selectively modulates the activity of a PDE4D2 polypeptide is designed. In one embodiment, the PDE4D2 catalytic domain polypeptide comprises the amino acid sequence of SEQ ID NO: 4. In another embodiment, the crystallized PDE4D2 catalytic domain polypeptide is in an orthorhombic crystalline form. In another embodiment, the three-dimensional structure of the crystallized PDE4D2 catalytic domain polypeptide in complex with a ligand can be determined to a resolution of about 2.3 Å or better.

Optionally, the present method can further comprise contacting a PDE4D2 catalytic domain polypeptide with the potential ligand and a ligand; and assaying the PDE4D2 catalytic domain polypeptide for binding of the potential ligand, for a change in activity of the PDE4D2 catalytic domain polypeptide, or both.

The presently disclosed subject matter also provides a method for screening a plurality of compounds for a ligand of a PDE4D2 catalytic domain polypeptide comprising (a) providing a library of test samples; (b) contacting a crystalline form comprising a PDE4D2 polypeptide in complex with a ligand with each test sample; (c) detecting an interaction between a test sample and the crystalline PDE4D2 polypeptide in complex with a ligand; (d) identifying a test sample that interacts with the crystalline PDE4D2 polypeptide in complex with a ligand; and (e) isolating a test sample that interacts with the crystalline PDE4D2 polypeptide in complex with a ligand, whereby a plurality of compounds is screened for a ligand of a PDE4D2 catalytic domain polypeptide. In one embodiment, the PDE4D2 polypeptide comprises a PDE4D2 catalytic domain. In another embodiment, the PDE4D2 polypeptide is a human PDE4D2 polypeptide. In another embodiment, the PDE4D2 polypeptide comprises the amino acid sequence of SEQ ID NO: 4. In one embodiment, the library of test samples is bound to a substrate. In another embodiment, the library of test samples is synthesized directly on a substrate.

VIII. PDE4D2 Polypeptides

The generation of mutant and chimeric PDE4D2 polypeptides is also an aspect of the presently disclosed subject matter. A chimeric polypeptide can comprise a PDE4D2 CD polypeptide or a portion of a PDE4D2 CD, (i.e. a PDE4D2 CD) which is fused to a candidate polypeptide or a suitable region of the candidate polypeptide. Throughout the present disclosure it is intended that the term “mutant” encompass not only mutants of a PDE4D2 CD polypeptide but chimeric proteins generated using a PDE4D2 CD as well. It is thus intended that the following discussion of mutant PDE4D2 CDs apply mutatis mutandis to chimeric PDE4D2 and PDE4D2 CD polypeptides and to structural equivalents thereof.

In accordance with the presently disclosed subject matter, a mutation can be directed to a particular site or combination of sites of a wild-type PDE4D2 CD. For example, an accessory binding site or the binding pocket can be chosen for mutagenesis. Similarly, a residue having a location on, at or near the surface of the polypeptide can be replaced, resulting in an altered surface charge of one or more charge units, as compared to the wild-type PDE4D2 and PDE4D2 CD. Alternatively, an amino acid residue in a PDE4D2 or a PDE4D2 CD can be chosen for replacement based on its hydrophilic or hydrophobic characteristics.

Such mutants can be characterized by any one of several different properties as compared with the wild-type PDE4D2 CD. For example, such mutants can have an altered surface charge of one or more charge units, or can have an increase in overall stability. Other mutants can have altered substrate specificity in comparison with, or a higher specific activity than, a wild type PDE4D2 or PDE4D2 CD.

PDE4D2 and PDE4D2 CD mutants of the presently disclosed subject matter can be generated in a number of ways. For example, the wild-type sequence of a PDE4D2 or a PDE4D2 CD can be mutated at those sites identified using presently disclosed subject matter as desirable for mutation by employing oligonucleotide-directed mutagenesis or other conventional methods. Alternatively, mutants of a PDE4D2 or a PDE4D2 CD can be generated by the site-specific replacement of a particular amino acid with an unnaturally occurring amino acid. In addition, PDE4D2 or PDE4D2 CD mutants can be generated through replacement of an amino acid residue, for example, a particular cysteine or methionine residue, with selenocysteine or selenomethionine. This can be achieved by growing a host organism capable of expressing either the wild type or mutant polypeptide on a growth medium depleted of either natural cysteine or methionine (or both) but enriched in selenocysteine or selenomethionine (or both).

Mutations can be introduced into a DNA sequence coding for a PDE4D2 or a PDE4D2 CD using synthetic oligonucleotides. These oligonucleotides contain nucleotide sequences flanking the desired mutation sites. Mutations can be generated in the full-length DNA sequence of a PDE4D2 or a PDE4D2 CD or in any sequence coding for polypeptide fragments of a PDE4D2 or a PDE4D2 CD.

According to the presently disclosed subject matter, a mutated PDE4D2 or PDE4D2 CD DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired PDE4D2 or PDE4D2 CD mutant coding sequence, an expression vector includes control sequences encoding a promoter, operator, ribosome binding site, translation initiation signal, and, optionally, a repressor gene or various activator genes and a signal for termination. Where secretion of the produced mutant is desired, nucleotides encoding a “signal sequence” can be inserted prior to a PDE4D2 or a PDE4D2 CD mutant coding sequence. For expression under the direction of the control sequences, a desired DNA sequence is operatively linked to the control sequences; that is, the sequence has an appropriate start signal in front of the DNA sequence encoding the PDE4D2 or PDE4D2 CD mutant, and the correct reading frame to permit expression of that sequence under the control of the control sequences and production of the desired product encoded by that PDE4D2 or PDE4D2 CD sequence.

Any of a wide variety of well-known available expression vectors can be used to express a mutated PDE4D2 or PDE4D2 CD coding sequences of presently disclosed subject matter. These include for example, vectors consisting of segments of chromosomal, non-chromosomal, and synthetic DNA sequences, such as known derivatives of SV40, known bacterial plasmids, i.e., plasmids from E. coli including colE1, pCR1, pBR322, pMB9 and their derivatives, wider host range plasmids, i.e., RP4, phage DNAs, i.e., derivatives of phage λ, i.e., NM 989, and other DNA phages, i.e., M13 and filamentous single stranded DNA phages, yeast plasmids and vectors derived from combinations of plasmids and phage DNAs, such as plasmids which have been modified to employ phage DNA or other expression control sequences. In one embodiment of the presently disclosed subject matter, a vector amenable to expression in a pET-based expression system is employed. The pET expression system is available from Novagen, Inc. (Madison, Wis., United States of America).

In addition, any of a wide variety of expression control sequences—i.e. sequences that control the expression of a DNA sequence when operatively linked to it—can be used in these vectors to express the mutated DNA sequences according to presently disclosed subject matter. Such useful expression control sequences, include, but are not limited to the early and late promoters of SV40 for animal cells; the lac system, the trp system, the TAC or TRC system, the major operator and promoter regions of phage λ, and the control regions of fd coat protein for E. coli; the promoter for 3-phosphoglycerate kinase or other glycolytic enzymes, the promoters of acid phosphatase, (for example, Pho5), and the promoters of the yeast α-mating factors for yeast; as well as other sequences known to control the expression of genes of prokaryotic or eukaryotic cells or their viruses, and various combinations thereof.

A wide variety of hosts can be employed for producing mutated PDE4D2 and PDE4D2 CD polypeptides according to presently disclosed subject matter. These hosts include, for example, bacteria, such as E. coli, Bacillus, and Streptomyces; fungi, such as yeasts; animal cells, such as CHO and COS-1 cells; plant cells; insect cells, such as Sf9 cells; and transgenic host cells.

It should be understood that not all expression vectors and expression systems function in the same way to express mutated DNA sequences of presently disclosed subject matter, and to produce modified PDE4D2 and PDE4D2 CD polypeptides or PDE4D2 or PDE4D2 CD mutants. Neither do all hosts function equally well with the same expression system. One of skill in the art can, however, make a selection among these vectors, expression control sequences and hosts without undue experimentation and without departing from the scope of presently disclosed subject matter. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the DNA sequence encoding a modified PDE4D2 or PDE4D2 CD polypeptide of presently disclosed subject matter, with particular regard to the formation of potential secondary and tertiary structures.

Hosts should be selected by consideration of their compatibility with the chosen vector, the toxicity of a modified PDE4D2 or PDE4D2 CD to them, their ability to express mature products, their ability to fold proteins correctly, their fermentation requirements, the ease of purification of a modified PDE4D2 or PDE4D2 CD and safety. Within these parameters, one of skill in the art can select various vector/expression control system/host combinations that will produce useful amounts of a mutant PDE4D2 or PDE4D2 CD. A mutant PDE4D2 or PDE4D2 CD produced in these systems can be purified by a variety of conventional steps and strategies, including those used to purify the wild type PDE4D2 or PDE4D2 CD.

Once a PDE4D2 CD mutation(s) has been generated in the desired location, such as an active site or dimerization site, the mutants can be tested for any one of several properties of interest. For example, mutants can be screened for an altered charge at physiological pH. This is determined by measuring the mutant PDE4D2 or PDE4D2 CD isoelectric point (pl) and comparing the observed value with that of the wild-type parent. Isoelectric point can be measured by gel-electrophoresis according to the method of Wellner (Wellner, Anal Chem. 43:597, 1971). A mutant PDE4D2 or PDE4D2 CD polypeptide containing a replacement amino acid located at the surface of the enzyme, as provided by the structural information of presently disclosed subject matter, can lead to an altered surface charge and an altered pl.

VIII.A. Generation of an Engineered PDE4D2 CD or PDE4D2 CD Mutant

In an embodiment of the presently disclosed subject matter, a unique PDE4D2 or PDE4D2 CD polypeptide is generated. Such a mutant can facilitate purification and the study of the catalytic abilities of a PDE4D2 polypeptide.

As used in the following discussion, the terms “engineered PDE4D2”, “engineered PDE4D2 LDB”, “PDE4D2 mutant”, and “PDE4D2 CD mutant” refers to polypeptides having amino acid sequences which contain at least one mutation in the wild-type sequence. The terms also refer to PDE4D2 and PDE4D2 CD polypeptides which are capable of exerting a biological effect in that they comprise all or a part of the amino acid sequence of an engineered PDE4D2 or PDE4D2 CD polypeptide of the presently disclosed subject matter, or cross-react with antibodies raised against an engineered PDE4D2 or PDE4D2 CD polypeptide, or retain all or some or an enhanced degree of the biological activity of the engineered PDE4D2 or PDE4D2 CD amino acid sequence or protein. Such biological activity can include catalytic activity and the binding of small molecules in general.

The terms “engineered PDE4D2 CD” and “PDE4D2 CD mutant” also includes analogs of an engineered PDE4D2 CD or PDE4D2 CD polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct PDE4D2 CD or PDE4D2 CD mutant analogs. There is no need for a PDE4D2. CD or PDE4D2 CD mutant polypeptide to comprise all or substantially all of the amino acid sequence of SEQ ID NOs:2 or 4. Shorter or longer sequences can be employed in the presently disclosed subject matter; shorter sequences are herein referred to as “segments”. Thus, the terms “engineered PDE4D2 CD” and “PDE4D2 CD mutant” also include fusion, chimeric or recombinant PDE4D2 CD, or PDE4D2 CD mutant polypeptides and proteins comprising sequences of the presently disclosed subject matter. Methods of preparing such proteins are disclosed herein above and are known in the art.

VIII.A.1. Sequences that are Substantially Identical to a PDE4D2 or PDE4D2 CD Mutant Sequence of the Presently Disclosed Subject Matter

Nucleic acids that are substantially identical to a nucleic acid sequence of a PDE4D2 or PDE4D2 CD mutant of the presently disclosed subject matter, i.e. allelic variants, genetically altered versions of the gene, etc., bind to a PDE4D2 or PDE4D2CD mutant sequence under stringent hybridization conditions. By using probes, particularly labeled probes of DNA sequences, one can isolate homologous or related genes. The source of homologous genes can be any organism, including, but not limited to primates; rodents, such as rats and mice; canines; felines; bovines; equines; yeast; and nematodes.

Among mammalian species, i.e. human and mouse, homologs can have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. In one embodiment, a reference sequence is at least about 18 nucleotides (nt) long, in another embodiment at least about 30 nt long, and can extend to the complete sequence that is being compared. Algorithms for sequence analysis are known in the art, such as BLAST, described in Altschul et al., J Mol Biol 215:403-10, 1990.

Percent identity or percent similarity of a DNA or peptide sequence can be determined, for example, by comparing sequence information using the GAP computer program, available from the University of Wisconsin Genetics Computer Group (now part of Accelrys Inc, San Diego, Calif., United States of America). The GAP program utilizes the alignment method of Needleman et al., J Mol Biol, 48:443, 1970, as revised by Smith et al., Adv Appl Math, 2:482-89, 1981. Briefly, the GAP program defines similarity as the number of aligned symbols (i.e., nucleotides or amino acids) that are similar, divided by the total number of symbols in the shorter of the two sequences. Exemplary parameters for the GAP program are the default parameters, which do not impose a penalty for end gaps. See i.e., Schwartz et al., eds., Atlas of Protein Sequence and Structure, National Biomedical Research Foundation, pp. 357-358, 1979, and Gribskov et al., Nucl Acids Res, 14: 6745-63, 1986.

The term “similarity” is contrasted with the term “identity”. Similarity is defined as above; “identity”, however, refers to a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, i.e. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs: 1 or 3; or (b) the DNA analog sequence is capable of hybridization with DNA sequences of (a) under stringent conditions and which encode a biologically active PDE4D2 or PDE4D2 CD gene product; or (c) the DNA sequences are degenerate as a result of alternative genetic code to the DNA analog sequences defined in (a) and/or (b). Substantially identical analog proteins and nucleic acids will have in one embodiment between about 70% and 80%, in another embodiment between about 81% to about 90%, and in still another embodiment between about 91% and 99% sequence identity with the corresponding sequence of the native protein or nucleic acid. Sequences having lesser degrees of identity but comparable biological activity are considered to be equivalents.

As used herein, “stringent conditions” refers to conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA, and 15% formamide at 68° C. For the purposes of specifying additional conditions of high stringency, preferred conditions comprise a salt concentration of about 200 mM and temperature of about 45° C. One example of stringent conditions is hybridization in 4×SSC, at 65° C., followed by a washing in 0.1×SSC at 65° C. for one hour. Another exemplary stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

VIII.A.2. Complementarity and Hybridization to an Engineered PDE4D2 or PDE4D2 CD Mutant Sequence

As used herein, the term “functionally equivalent codon” is used to refer to codons that encode the same amino acid, such as the ACG and AGU codons for serine. PDE4D2 or PDE4D2 CD-encoding nucleic acid sequences comprising SEQ ID NOs:1 and 3, which have functionally equivalent codons are covered by the presently disclosed subject matter. Thus, when referring to the sequence examples presented in SEQ ID NOs:1 and 3, applicants contemplate substitution of functionally equivalent codons into the sequence example of SEQ ID NOs:1 and 3. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

VIII.B. Biological Equivalents

The presently disclosed subject matter envisions and includes biological equivalents of PDE4D2 or PDE4D2 CD mutant polypeptide of the presently disclosed subject matter. The term “biological equivalent” refers to proteins having amino acid sequences which are substantially identical to the amino acid sequence of a PDE4D2 CD mutant of the presently disclosed subject matter and which are capable of exerting a biological effect in that they are capable of binding a small molecule, binding a co-regulator, homo- or heterodimerizing or cross-reacting with anti-PDE4D2 or PDE4D2 CD mutant antibodies raised against a mutant PDE4D2 or PDE4D2 CD polypeptide of the presently disclosed subject matter.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the presently disclosed subject matter is not limited to a particular mechanism of action. It is thus in accordance with the presently disclosed subject matter that various changes can be made in the amino acid sequence of a PDE4D2 or PDE4D2 CD mutant polypeptide of the presently disclosed subject matter or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs:2 and 4, applicants envision substitution of codons that encode biologically equivalent amino acids, as described herein, into the sequence example of SEQ ID NOs:2 and 4, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, i.e. substitution of Ile for Leu. Changes designed by man can be introduced through the application of site-directed mutagenesis techniques, i.e., to introduce improvements to the antigenicity of the protein or to test a PDE4D2 or PDE4D2 CD mutant polypeptide of the presently disclosed subject matter in order to modulate co-regulator-binding or other activity, at the molecular level.

Amino acid substitutions, such as those which might be employed in modifying a PDE4D2 or PDE4D2 CD mutant polypeptide of the presently disclosed subject matter are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Those of skill in the art will appreciate other biologically functional equivalent changes. It is implicit in the above discussion, however, that one of skill in the art can appreciate that a radical, rather than a conservative substitution is warranted in a given situation. Non-conservative substitutions in mutant PDE4D2 or PDE4D2 CD polypeptides of the presently disclosed subject matter are also an aspect of the presently disclosed subject matter.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, J Mol Biol, 157:105-132, 1982, incorporated herein by reference). It is known that certain amino acids can be substituted for other amino acids having a similar hydropathic index or score and still retain a similar biological activity. In one embodiment, amino acids for which the hydropathic indices are within ±2 of the original value are chosen, in another embodiment those within ±1 of the original value are chosen, and in still another embodiment those within ±0.5 of the original value are chosen, in making amino acid changes based upon the hydropathic index.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101 to Hopp, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, in one embodiment amino acids whose hydrophilicity values are within ±2 of the original value are chosen, in another embodiment those that are within ±1 of the original value are chosen, and in still another embodiment those within ±0.5 of the original value are chosen, in making changes based upon similar hydrophilicity values.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that presently disclosed subject matter is not limited to the particular nucleic acid and amino acid sequences of SEQ ID NOs:1-4. Recombinant vectors and isolated DNA segments can therefore variously include a PDE4D2 or PDE4D2 CD mutant polypeptide-encoding region itself, include coding regions bearing selected alterations or modifications in the basic coding region, or include larger polypeptides which nevertheless comprise a PDE4D2 or PDE4D2 CD mutant polypeptide-encoding regions or can encode biologically functional equivalent proteins or polypeptides which have variant amino acid sequences. Biological activity of a PDE4D2 or PDE4D2 CD mutant polypeptide can be determined, for example, by employing binding assays known to those of skill in the art.

The nucleic acid segments of the presently disclosed subject matter, regardless of the length of the coding sequence itself, can be combined with other DNA sequences, such as promoters, enhancers, polyadenylation signals, additional restriction enzyme sites, multiple cloning sites, other coding segments, polyhistidine encoding segments and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs:1 and 3, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the presently disclosed subject matter encompass biologically functional equivalents of PDE4D2 or PDE4D2 CD mutant polypeptides. Such sequences can arise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, i.e., to introduce improvements to the antigenicity of the protein or to test variants of a PDE4D2 or PDE4D2 CD mutant of the presently disclosed subject matter in order to examine the degree of lipid-binding activity, or other activity at the molecular level. Various site-directed mutagenesis techniques are known to those of skill in the art and can be employed in the presently disclosed subject matter.

The presently disclosed subject matter further encompasses fusion proteins and peptides wherein a PDE4D2 or PDE4D2 CD mutant coding region of the presently disclosed subject matter is aligned within the same expression unit with other proteins or peptides having desired functions, such as for purification or immunodetection purposes.

Recombinant vectors form important further aspects of the presently disclosed subject matter. Particularly useful vectors are those in which the coding portion of the DNA segment is positioned under the control of a promoter. The promoter can be that naturally associated with a PDE4D2 gene, as can be obtained by isolating the 5′ non-coding sequences located upstream of the coding segment or exon, for example, using recombinant cloning and/or polymerase chain reaction (PCR) technology and/or other methods known in the art, in conjunction with the compositions disclosed herein.

In other embodiments, certain advantages can be gained by positioning the coding DNA segment under the control of a recombinant, or heterologous, promoter. As used herein, a recombinant or heterologous promoter is a promoter that is not normally associated with a PDE4D2 gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (see e.g., Sambrook et al., (1989) Molecular Cloning: A Laboratory Manual, Cold Spring Harbor Laboratory (2^{nd} ed.), New York, specifically incorporated herein by reference). The promoters employed can be constitutive or inducible and can be used under the appropriate conditions to direct high level expression of the introduced DNA segment, such as is advantageous in the large-scale production of recombinant proteins or peptides. One representative promoter system contemplated for use in high-level expression is a T7 promoter-based system.

IX. The Role of the Three-Dimensional Structure of the PDE4D2 LDB in Solving Additional PDE4D2 Crystals

Because polypeptides can crystallize in more than one crystal form, the structural coordinates of a PDE4D2 CD, or portions thereof, in complex with a co-regulator as provided by the presently disclosed subject matter, are particularly useful in solving the structure of other crystal forms of PDE4D2 and the crystalline forms of other PDEs. The coordinates provided in the presently disclosed subject matter can also be used to solve the structure of PDE4D2 or PDE4D2 CD mutants (such as those above), PDE4D2 LDB co-complexes, or the crystalline form of any other protein with significant amino acid sequence homology to any functional domain of PDE4D2.

One method that can be employed for the purpose of solving additional PDE4D2 crystal structures is molecular replacement. See generally, Rossmann, ed., The Molecular Replacement Method, Gordon & Breach, New York, 1972. In the molecular replacement method, an unknown crystal form, whether it is another crystal form of a PDE4D2 or a PDE4D2 CD, (i.e. a PDE4D2 or a PDE4D2 CD mutant), a PDE4D2 or a PDE4D2 CD polypeptide in complex with another compound (i.e. a “co-complex”) or the crystal of some other protein with significant amino acid sequence homology to any functional region of the PDE4D2 CD (i.e. another PDE), can be determined using the PDE4D2 CD structure coordinates provided in Tables 4-5. This method provides an accurate structural form for the unknown crystal more quickly and efficiently than attempting to determine such information ab initio.

In addition, in accordance with presently disclosed subject matter, PDE4D2 or PDE4D2 CD mutants can be crystallized in complex with known modulators, such as a co-regulator. The crystal structures of a series of such complexes can then be solved by molecular replacement and compared with that of wild-type PDE4D2 or the wild-type PDE4D2 CD. Potential sites for modification within the various binding sites of the enzyme can thus be conveniently identified. This information provides an additional tool for identifying efficient binding interactions, for example, increased hydrophobic interactions between the PDE4D2 CD and a chemical entity or compound.

All of the complexes referred to in the present disclosure can be studied using X-ray diffraction techniques (See i.e., Blundell & Johnson, Meth Enzymol, 114A & 115B, Wyckoff et al., eds., Academic Press, 1985) and can be refined using computer software, such as the X-PLOR™ program (Brünger, X-PLOR, Version 3.1. A System for X-ray Crystallography and NMR, Yale University Press, New Haven, Conn., United States of America, 1992b; X-PLOR is available from Accelrys Inc, San Diego, Calif., United States of America). This information can thus be used to optimize known classes of PDE4D2 and PDE4D2 CD ligands, and more importantly, to design and synthesize novel classes of PDE4D2 and PDE4D2 CD ligands, including co-regulators.

### EXAMPLES

The following Examples have been included to illustrate exemplary modes of the presently disclosed subject matter. Certain aspects of the following Examples are described in terms of techniques and procedures found or contemplated by the present inventors to work well in the practice of the presently disclosed subject matter. These Examples are exemplified through the use of standard laboratory practices of the inventors. In light of the present disclosure and the general level of skill in the art, those of skill will appreciate that the following Examples are intended to be exemplary only and that numerous changes, modifications, and alterations can be employed without departing from the spirit and scope of the presently disclosed subject matter.

### Example 1

### Protein Expression and Purification

The EST (expressed sequence tag) cDNA clone of PDE4D2 (BF059733) was purchased from the American Type Culture Collection (ATCC). The protein expression and purification of the catalytic domain of PDE4D2 (amino acids 79-438) was described previously (Huai et al., 2003). Briefly, the EST cDNA clones of PDE4D2 (BF059733) were purchased from ATCC and subcloned following standard methods. The coding regions for amino acids 79-438 of PDE4D2 were amplified by PCR and subcloned into the expression vector pET15b. The resulting plasmid pET-PDE4D2 was transformed into E. coli strain BL21-CODONPLUS® (Stratagene, Inc., La Jolla, Calif., United States of America) for overexpression. The E. coli cell carrying pET-PDE4D2 was grown in LB medium at 37° C. to absorption OD₆₀₀=0.7 and then 0.1 mM isopropyl β-D-thiogalactopyranoside was added for further growth at 12° C. for 40 hours. The recombinant PDE4D2 was purified by Ni-NTA affinity column (Qiagen Inc., Valencia, Calif., United States of America), thrombin cleavage, Q-SEPHAROSE™ (available from Amersham Biosciences Corp., Piscataway, N.J., United States of America) and SUPERDEX 200™ (available from Amersham Biosciences Corp., Piscataway, N.J., United States of America) columns. The PDE4D2 protein had a purity of greater than 95% as shown in by SDS-PAGE and was apparently a dimer as judged on the basis of the molecular sieving column. A typical purification yielded over 100 mg PDE4D2 from a 2 liter cell culture.

### Example 2

### Crystallization and Data Collection

The crystals were grown by vapor diffusion against a well buffer of 50 mM HEPES (pH 7.5), 15% PEG3350, 25% ethylene glycol, 5% methanol, and 5% DMSO at 4° C. The protein drop was prepared by mixing 10 mM cAMP and 0.4 mM zinc sulfate with 15 mg/mL PDE4D2 in a storage buffer of 50 mM NaCl, 20 mM Tris-HCl (pH 7.5), and 1 mM β-mercaptoethanol for the crystallization. To saturate the cAMP binding, the crystals were soaked in a buffer of 50 mM HEPES (pH 7.5), 20% PEG3350, 25% ethylene glycol, 0.4 mM zinc sulfate, and 50 mM cAMP at room temperature for 5 hours and then immediately dipped into liquid nitrogen. The crystals of PDE4D2 have the space group P2₁2₁2₁ with cell dimensions of a=99.2 Å, b=111.2 Å, and c=159.7 Å. The diffraction data were collected on beamline 14C of APS at Argonne National Laboratory (Table 3) and processed by program HKL (Otwinowski and Minor, 1997).

### Example 3

### Structure Determination and Refinement

The structure of PDE4D2 in complex with AMP was solved by the direct application of the tetramer of the PDE4D2-rolipram structure to the crystal system (Huai et al., 2003). The orientation of the individual subunits in the PDE4D2-AMP tetramer was optimized by rigid-body refinement of CNS (Brünger, 1998). The electron density map was improved by the density modification package of CCP4 (1994). The atomic model was rebuilt by program O (Jones et al., 1991) and refined by CNS. See Table 3 for a summary of the statistics of the structure.

LENGTHY TABLE REFERENCED HERE
US20070015270A1-20070118-T00001
Please refer to the end of the specification for access instructions.

LENGTHY TABLE REFERENCED HERE
US20070015270A1-20070118-T00002
Please refer to the end of the specification for access instructions.

LENGTHY TABLE REFERENCED HERE
US20070015270A1-20070118-T00003
Please refer to the end of the specification for access instructions.

LENGTHY TABLE REFERENCED HERE
US20070015270A1-20070118-T00004
Please refer to the end of the specification for access instructions.

LENGTHY TABLE REFERENCED HERE
US20070015270A1-20070118-T00005
Please refer to the end of the specification for access instructions.

### REFERENCES

The references listed below as well as all references cited in the specification are incorporated herein by reference to the extent that they supplement, explain, provide a background for or teach methodology, techniques and/or compositions employed herein.
Adams P D, Pannu N S, Read R J, Brunger A T, 1997, Proc Natl Acad Sci USA 94(10): 5018-23
Agrawal S (ed.) Methods in Molecular Biology, volume 20, Humana Press, Totowa, N.J., United States of America.
Allen et al., 1973, J. Chem. Doc. 13: 119
Altschul et al., J Mol Biol 215:403-10, 1990
Amemiya, Y, 1997, Methods in Enzymology, Vol. 276, Academic Press, San Diego, Calif., United States of America, pp. 233-243
Antoni, F. (2000) Molecular diversity of cyclic AMP signaling, Front. Neuroendocrin., 21, 103-132.
Appelt, 1993, Perspectives in Drug Discovery and Design 1: 23-48
Atienza, J. M., Susanto, D., Huang, C., McCarty, A. S., and Colicelli, J. (1999) Identification of inhibitor specificity determinants in a mammalian phosphodiesterase. J. Biol. Chem. 274, 4839-4847.
Ausubel F M, Brent R, Kingston R E, Moore D D, Seidman J G, Smith J A & Struhl K, eds (1989) Current Protocols in Molecular Biology. Wiley, New York.
Barnette, M. S., and Underwood, D.C. (2000). New phosphodiesterase inhibitors as therapeutics for the treatment of chronic lung disease. Curr. Opin. Pulmonary Med., 6, 164-169.
Bartlett et al., 1989, Spec. Publ., Roy. Soc. Chem. 78: 182-196
Blundell & Johnson, Meth Enzymol, 114A & 115B, Wyckoff et al., eds., Academic Press, 1985
Bohm, J Comput Aid Mol Des, 6: 61-78, 1992
Boobbyer et al. (1989) J Med Chem 32:1083-1094
Bowie et al., 1991, Science 253: 164-170.
Brint et al., 1987, J Mol Graph 5:49-56
Brooks et al., J Comp Chem, 8: 132, 1993
Brünger A T (1992) Nature 355: 472-475.
Brünger A T (1992b) X-PLOR, Version 3.1. A System for X-ray Crystallography and NMR, Yale University Press, New Haven, Conn., United States of America
Brünger et al. (1998) Acta Cryst., D54, 905-921.
Bugg et al., Scientific American, December 1993: 92-98
Bundell et al., 1988, Eur. J. Biochem. 172: 513
Callahan, S. M., Cornell, N. W., & Dunlap, P. V. (1995) Purification and properties of periplasmic 3′,5′-cyclic nucleotide phosphodiesterase, J. Biol. Chem., 270, 17627-17632.
Case et al., AMBER 5, University of California, San Francisco, 1997
Cohen et al., 1990, J. Med. Chem. 33: 883-894
Collaborative Computational Project, Number 4. 1994. “The CCP4 Suite: Programs for Protein Crystallography”. Acta Cryst. D50, 760-763.
Conti, M. & Jin, S. L. (1999) The molecular biology of cyclic nucleotide phosphodiesterases, Prog. Nucleic Acid Res. Mol. Biol., 63, 1-38.
Corbin, J. D. & Francis, S. H. (1999) Cyclic GMP phosphodiesterase-5: target of sildenafil, J. Biol. Chem., 274, 13729-13732.
Corbin, J. D. and Francis, S. H. (2002). Pharmacology of phosphodiesterase-5 inhibitors. Int. J. Clin. Pract. 56, 453-459.
Creighton, (1983) Proteins: Structures and Molecular Principles, W.H. Freeman & Co., New York.
Cwirla et al., 1990, Proc. Natl. Acad. Sci. USA, 87: 6378-6382
Dauter Z, Li M, Wlodawer A., Acta Crystallogr D 2001; 57: 239-49
DesJarlais 1988, J. Med. Chem. 31: 722-729
DesJarlais et al., 1986, J. Med. Chem. 29: 2149-2153
Devlin et al., 1990, Science, 249: 404-406
Ducruix and Geige 1992, Crystallization of Nucleic Acids and Proteins: A Practical Approach, IRL Press, Oxford, England.
Dunbrack et al., (1997) Folding & Design, 2: 27-42.
Dym, O., Xenarios, I., Ke, H., & Colicelli, J. (2002) Molecular docking of competitive phosphodiesterase inhibitors, Mol. Pharmacol., 61, 20-25.
Ebel et al., (1992) Biochem. 31:12083.
Eisen et al., Proteins, 19:199-221, 1994
Erickson, 1993, Perspectives in Drug Discovery and Design 1: 109-128
Fitzgerald P M D, 1988 J. Appl. Cryst., Vol. 21, pp. 273-278.
Francis, S. H., Colban, J. L., McAllister-Lucas, L. M., & Corbin, J. D., (1994) Zinc interactions and conserved motifs of the cGMP-binding cGMP-specific phosphodiesterase suggest that it is a zinc hydrolase, J. Boil. Chem., 269, 22477-22480.
Francis, S. H, Turko, I. V., and Corbin, J. D. (2001) Cyclic nucleotide phosphodiesterases: relating structure and function. Prog. Nucleic Acid Res. Mol. Biol. 65, 1-52.
Giembycz, M. A. (2000) Phosphodiesterase 4 inhibitors and the treatment of asthma, Drugs, 59, 193-212.
Giembycz, M. A. (2002). Development status of second generation PDE 4 inhibitors for asthma and COPD: the story so far. Monaldi Arch. Chest Dis. 57, 48-64.
Goeddel (1990) Gene Expression Technology: Methods in Enzymology, Academic Press, San Diego, Calif., United States of America.
Goodford et al., 1985, J. Med. Chem. 28: 849-857
Goodsell & Olsen, Proteins, 8:195-202, 1990
Greer, 1985, Science 228: 1055
Gribskov et al., Nucl Acids Res, 14: 6745-63, 1986
Gutberlet T, Heinemann U & Steiner M, Acta Crystallogr D 2001, 57: 349-54
Hardman, J. G., Beavo, J. a., Gray, J. P., Chrisman, T. D., Patterson, W. D., & Sutherland, E. W. (1971) Ann. N.Y. Acad. Sci., 185, 27-35.
Hauptman, Curr Opin Struct Biol 7: 672-80, 1997
Hendrickson W A, Trends Biochem Sci 2000 December; 25(12): 637-43
Hendrickson W A & Ogata C M, 1997, Methods in Enzymology, Vol. 276, San Diego, Calif., United States of America, pp. 494-523
Houslay, M. D. & Milligan, G. (1997) Tailoring cAMP-signalling responses through isoform multiplicity, TIBS, 22, 217-224.
Houslay, M. D., Sullivan, M., & Bolger, G. B. (1998) The multienzyme PDE4 cyclic adenosine monophosphate-specific phosphodiesterase family: intracellular targeting, regulation, and selective inhibition by compounds exerting anti-inflammatory and antidepressant actions, Adv. Pharmocol., 44, 225-343.
Houslay, M. D. (1998) Adaptation in cyclic AMP signaling processes: a central role for cyclic AMP phosphodiesterases, Cell & Develop. Biol., 9, 161-167.
Huai, Q., Kim, H. Y., Liu, Y., Zhao, Y., Mondragon, A., Liu, J., & Ke, H. (2002) Crystal Structure of Calcineurin-Cyclophilin-Cyclosporin Shows Common but Distinct Recognition of Immunophilin-Drug Complexes, Proc. Natl. Acad. Sci., USA, 99, 12037-12042.
Huang, Z., Ducharme, Y., Macdonald, D., & Robichaud, A. (2001) The next generation of PDE4 inhibitors, Curr. Opin. Chem. Biol., 5, 432-438.
Jacobitz, S., McLaughlin, M. M., Livi, G. P., Bauman, M., & Torphy, T. J. (1996) Mapping the functional domains of human recombinant phosphodiesterase 4A: Structure requirements for catalytic activity and rolipram binding, Mol. Pharmacol. 50, 891-899.
Jacobitz, S., Ryan, M. D., McLaughlin, M. M., Livi, G. P., Dewolf, Jr., W. E., and Torphy, T. J. (1997) Role of conserved histidines in catalytic activity and inhibitor binding of human recombinant phosphodiesterase 4A. Mol. Pharmacol. 51, 999-1006.
Jakes et al., 1986, J Mol Graph 4:12-20
Jakes et al., 1987 J Mol Graph 5:41-48
Jancarik, J & Kim, S. H., J. Appl. Cryst. 1991 24: 409-411
Jin, S. L., Swinnen, J. V., and Conti, M. (1992) Characterization of the structure of a low Km, rolipram-sensitive cAMP phosphodiesterase. J. Biol. Chem. 267, 18929-18939.
Jones, T. A, Zou, J-Y., Cowan, S. W. & Kjeldgaard, M. (1991) Acta Cryst. A47, 110-119.
Kahn R & Fourme R, 1997, Methods in Enzymology, Vol. 276, Academic Press, San Diego, Calif., United States of America, pp. 268-286
Klein C. (2002) Nitric oxide and the other cyclic nucleotide. Cellular Signalling. 14, 493-8.
Kleywegt G J & Brunger A T, Structure 1996 4(8): 897-904
Knighton et al., 1992, Science 258
Kuntz et al., 1982, J. Mol. Biol. 161: 269-288
Kyte & Doolittle, J Mol Biol, 157:105-132, 1982
Lam et al., 1994, Science 263: 380-384
Lambert, in Practical Application of Computer-Aided Drug Design, Charifson, ed. Marcel-Dekker, New York, pp. 243-303, 1997
Laskowski et al., 1993, J. Appl. Cryst. 26:283-291.
Lattman E E (1985) Meth Enzymol, 115:55-77.
Lattman E E (1996) Proteins 25: i-ii.
Lawrence et al., 1992, Proteins 12:31-41
Levitt D G, Acta Crystallogr. D 2001 57: 1013-9
Lohse, D. L., Denu, J. M., & Dixon, J. E. (1995) Structure, 3, 987-990.
Lucas K A. Pitari G M. Kazerounian S. Ruiz-Stewart I. Park J. Schulz S. Chepenik K P. Waldman S A. (2000) Guanylyl cyclases and signaling by cyclic GMP. Pharmacological Reviews. 52, 375-414.
Luthy R et al., 1992, Nature 356: 83-85.
Manganiello, V. C., Taira, M., Degerman, F. & Belfrage, P. (1995) Type III cGMP-inhibited cyclic nucleotide phosphodiesterase (PDE3 gene family), Cell Signal., 7, 445-455.
Martin, J Med Chem 35:2145-54, 1992
McCree et al., 1992, J. Mol. Graphics 10: 44-46.
McPherson, 1982, Preparation and Analysis of Protein Crystals, John Wiley, New York.
McPherson, 1990, Eur. J. Biochem. 189:1-23.
McRee, Practical Protein Crystallography, Academic Press, New York, 1993
Mehats, C. M., Anderson, C. B., Filopanti, M., Jin, S. L. C., & Conti, M. (2002) Cyclic nucleotide phosphodiesterases and their role in endocrine cell signaling, Trends in Endocrin. Metabolism, 13, 29-35.
Miranker & Karplus, Proteins, 11:29-34, 1991
Müller, T., Engels, P., & Fozard, J. R. (1996) Subtypes of type 4 cAMP phosphodiesterase: structure, regulation and selective inhibition, TIPS, 17, 294-298.
Murshudov G N, Vagin A A, & Dodson E J, 1997, Acta Cryst. D Biol Crystallogr 53: 247-255
Navaza, 1994, Acta Crystallogr. A50:157-163.
Navia M A & Murcko M A, 1992, Current Opinions in Structural Biology 2: 202-210.
Needleman S B & Wunsch C D (1970) A General Method Applicable to the Search for Similarities in the Amino Acid Sequence of Two Proteins. J Mol Biol 48:443-453.
Nishibata & Itai, Tetrahedron, 47:8985
Omburo, G. A., Jacobitz, S., Torphy, T. J., & Colman, R. W. (1998) Critical role of conserved histidine pairs HNXXH and HDXXH in recombinant human phosphodiesterase 4A, Cell Signal., 10, 491-497.
Otwinowski, Z. & Minor, W. (1997) Processing of X-ray diffraction data collected in oscillation mode, Methods Enzymol., 276, 307-326.
Pearlman et al., Comput Phys Commun, 91: 1-41, 1995
Pearson W R & Lipman D J (1988) Improved Tools for Biological Sequence Comparison. Proc Natl Acad Sci USA 85:2444-2448.
Percival, M. D., Yeh, B., & Falgueyret, J. P. (1997) Zinc dependent activation of cAMP-specific phosphodiesterase (PDE4A), Biochem. Biophys. Res. Commun. 241, 175-180.
Perrakis A, Morris R, & Lamzin, V S, May 1999, Nature Structural Biology 6: 458-463
Petsko G A, 1985, Methods in Enzymology, Vol. 114, Academic Press, Orlando, Fla., United States of America, pp. 147-156
Piaz, V. D. and Giovannoni, P. (2000). Phosphodiesterase 4 inhibitors, structurally unrelated to rolipram, as promising agents for the treatment of asthma and other pathologies. Eur. J. Med. Chem. 35, 463-480.
Pillai, R., Kytle, K., Reyes, A., and Colicelli, J. (1993) Use of a yeast expression system for the isolation and analysis of drug-resistant mutants of a mammalian phosphodiesterase. PNAS 90, 11970-11974.
Rarey et al., J Comput Aid Mol Des, 10:41-54, 1996
Reilly, M. P., and Mohler, E. R. III (2001). Cilostazol: treatment of intermittent claudication. Annuals Pharmacotherapy. 35, 48-56.
Richter, W., Unciuleac, L., Hermsdorf, T., Kronbach, T., and Dettmer, D. (2001) Identification of inhibitor binding sites of the cAMP-specific phosphodiesterase 4. Cell. Signalling. 13, 287-297.
Rossmann (ed.), (1972) The Molecular Replacement Method, Gordon & Breach, New York.
Rotella, D. P. (2002). Phosphodiesterase 5 inhibitors: current status and potential applications. Nature Rev. Drug Discov. 1, 674-682.
Sambrook J & Russell D W (2001) Molecular Cloning: A Laboratory Manual, 3rd ed. Cold Spring Harbor Laboratory Press, Cold Spring Harbor, N.Y.
Schulz G E and R H Schirmer., Principles of Protein Structure, Springer-Verlag.
Schwartz et al., eds., Atlas of Protein Sequence and Structure, National Biomedical Research Foundation, pp. 357-358, 1979
Scott & Smith, 1990, Science, 249: 386-390
Sheldrick, Acta Cryst, A46: 467, 1990
Smith et al., Adv Appl Math, 2:482-89, 1981
Soderling, S. H., & Beavo, J. A. (2000) Regulation of cAMP and cGMP signaling: new phosphodiesterases and new functions, Curr. Opin. Cell Biol., 12, 174-179.
Souness, J. E., Aldous, D., & Sargent, C. (2000) Immunosuppressive and anti-inflammatory effects of cyclic AMP phosphodiesterase (PDE) type 4 inhibitors, Immunopharmacol., 47, 127-162.
Spina, D. (2003) Theophylline and PDE4 inhibitors in asthma. Curr. Opin. Pulm. Med. 9, 57-64.
Stoddard B L, Curr. Opin. Struct Biol 1998 October; 8(5): 612-8
Stork P J. Schmitt J M. (2002) Crosstalk between cAMP and MAP kinase signaling in the regulation of cell proliferation. Trends in Cell Biol. 12, 258-266.
Sturton, G. and Fitzgerald, M. (2002). Phosphodiesterase 4 inhibitors for the treatment of COPD. Chest 121, 192s-196s.
Terwilliger, T. C. and J. Berendzen. Acta Crystallographica D55:849-861, 1999
Thompson, W. J. (1991) Cyclic nucleotide phosphodiesterases: pharmacology, biochemistry and function, Pharmac. Ther., 51, 13-33.
Tibanyenda N et al., (1984) Eur. J. Biochem. 139:19.
Tijssen (1993) Laboratory Techniques in Biochemistry and Molecular Biology-Hybridization With Nucleic Acid Probes, Elsevier, New York;
Torphy, T. J. (1998) Phosphodiesterase isozymes: molecular targets for novel antiasthma agents, Am. J. Respir. Crit. Care Med., 157, 351-370.
Turko, I. V., Francis, S. H., and Corbin, J. D. (1998) Potential roles of conserved amino acids in the catalytic domain of the cGMP-binding cGMP-specific phosphodiesterase (PDE5). J. Biol. Chem. 273, 6460-6466.
Navia & Murcko, Curr Opin Struct Biol, 2:202-10, 1992
U.S. Pat. No. 4,554,101
U.S. Pat. No. 6,008,033
Vallee, B. L. & Auld, D. S. (1990) Zinc coordination, function, and structure of zinc enzymes and other proteins, Biochemistry, 29, 5647-5659.
Villieux F M D & Read R J, 1997, Methods in Enzymology, Vol. 277, Academic Press, Orlando, Fla., United States of America, pp. 18-52
Weber, 1991, Adv. Protein Chem. 41: 1-36
Weeks et al., Acta Crystallogr D49: 179, 1993
Weeks C M, DeTitta G T, Hauptman H A, Thuman P, Miller R, Acta Crystallogr A 1994; 50: 210-20
West et al., TIPS, 16:67-74 (1995)
Westbrook E M & Naday I, 1997, Methods in Enzymology, Vol. 276, Academic Press, San Diego, Calif., United States of America, pp. 244-268
Wlodawer et al., 1993, Ann. Rev. Biochem. 62: 543-585
Xu, R. X., Hassell, A. M., Vanderwall, D., Lambert, M. H., Holmes, W. D., Luther, M. A., Rocque, W. J., Milburn, M. V., Zhao, Y., Ke, H., & Nolte, R. T. (2000) Atomic structure of PDE4: Insight into phosphodiesterase mechanism and specificity, Science, 288, 1822-1825.
Zhang K Y J, Cowtan K, & Main P, 1997, Methods in Enzymology, Vol. 277, Academic Press, Orlando, Fla., United States of America, pp. 53-64
Zhang, W., Ke, H., Tretiakova, A. P., Jameson, B., & Colman, R. W. (2001) Identification of overlapping but distinct cAMP and cGMP binding sites of cyclic nucleotide phosphodiesterase 3A by site-directed mutagenesis and molecular modeling based on crystalline PDE4B, Protein. Sci., 10, 1481-1489.
Zhao, A. Z., Yan, C., Sonnenburg, W. K., & Beavo, J. A. (1997) Recent advances in the study of Ca 2+/Cam-activated phosphodiesterases, Signal Transduction in Health and Disease, 31, 237-251.

It will be understood that various details of the presently disclosed subject matter may be changed without departing from the scope of the presently disclosed subject matter. Furthermore, the foregoing description is for the purpose of illustration only, and not for the purpose of limitation, the presently disclosed subject matter being defined by the claims.

LENGTHY TABLE
The patent application contains a lengthy table section. A copy of the table is available in electronic form from the USPTO web site (http://seqdata.uspto.gov/?pageRequest=docDetail&DocID=US20070015270A1) An electronic copy of the table will also be available from the USPTO upon request and payment of the fee set forth in 37 CFR 1.19(b)(3).

What is claimed is:

1. A crystalline form comprising a substantially pure phosphodiesterase 4D2 (PDE4D2) polypeptide.

2. The crystalline form of claim 1, wherein the substantially pure phosphodiesterase 4D2 (PDE4D2) catalytic domain polypeptide is in complex with a ligand.

3. The crystalline form of claim 2, wherein the crystalline form has unit cell a=99.2 Å; b=111.2 Å; c=159.7 Å and space group P2₁2₁2₁.

4. The crystalline form of claim 2, wherein the crystalline form comprises four phosphodiesterase 4D2 (PDE4D2) catalytic domain polypeptides.

5. The crystalline form of claim 2, wherein the phosphodiesterase 4D2 (PDE4D2) catalytic domain polypeptide has the amino acid sequence shown in SEQ ID NO: 4.

6. The crystalline form of claim 2, wherein the complex has a crystalline structure further characterized by the coordinates corresponding to one of Table 4 and Table 5.

7. A binding site in a human phosphodiesterase 4D2 (PDE4D2) catalytic domain polypeptide for a substrate, wherein the substrate is in van der Waals, hydrogen bonding, or both van der Waals and hydrogen bonding contact with at least one of the following residues of the human phosphodiesterase 4D2 (PDE4D2) polypeptide: Tyr159, His160, His164, His200, Asp201, Met273, Asp318, Leu319, Asn321, Thr333, Ile336, Phe340, Gln369, and Phe372.

8. The binding site of claim 7, comprising four phosphodiesterase 4D2 (PDE4D2) catalytic domain polypeptides.

9. The binding site of claim 8, wherein at least two of the four phosphodiesterase 4D2 (PDE4D2) catalytic domain polypeptides are in van der Waals, hydrogen bonding, or both van der Waals and hydrogen bonding contact through at least one of the following residues: Arg116, Met147, Thr148, Asp151, Asn214, Thr215, Asn216, Glu218, Ala220, Leu221, Met222, Tyr223, Asn224, Asp225, Asn231, Leu234, Ala235, Lys239, Gln242, Glu243, Glu244, Lys254, Arg257, Gln258, Arg261, Ile265, Arg346, Glu349, and Arg350.

10. The binding site of claim 7, further comprising a metal ion.

11. A complex of a human phosphodiesterase 4D2 (PDE4D2) catalytic domain polypeptide and a substrate, wherein the substrate is in van der Waals, hydrogen bonding, or both van der Waals and hydrogen bonding contact with at least one of the following residues of the human phosphodiesterase 4D2 (PDE4D2) polypeptide: Tyr159, His160, His164, His200, Asp201, Met273, Asp318, Leu319, Asn321, Thr333, Ile336, Phe340, Gln369, and Phe372.

12. The complex of claim 11, comprising four phosphodiesterase 4D2 (PDE4D2) catalytic domain polypeptides and wherein at least two of the four phosphodiesterase 4D2 (PDE4D2) catalytic domain polypeptides are in van der Waals, hydrogen bonding, or both van der Waal and hydrogen bonding contact through one or more of the following residues: Arg116, Met147, Thr148, Asp151, Asn214, Thr215, Asn216, Glu218, Ala220, Leu221, Met222, Tyr223, Asn224, Asp225, Asn231, Leu234, Ala235, Lys239, Gln242, Glu243, Glu244, Lys254, Arg257, Gln258, Arg261, Ile265, Arg346, Glu349, and Arg350.

13. The complex of claim 11, further comprising a metal ion.

14. A crystal of the complex of claim 11.

15. A method for identifying a phosphodiesterase ligand, the method comprising:
a) providing atomic coordinates of a phosphodiesterase 4D2 (PDE4D2) catalytic domain in complex with a ligand to a computerized modeling system; and
b) modeling a ligand that fits spatially into the binding site of the phosphodiesterase 4D2 (PDE4D2) catalytic domain to thereby identify a phosphodiesterase ligand.

16. The method of claim 15, wherein the phosphodiesterase 4D2 (PDE4D2) catalytic domain comprises the amino acid sequence of SEQ ID NO: 4.

17. The method of claim 15, wherein the method further comprises identifying in an assay for phosphodiesterase-mediated activity a modeled ligand that increases or decreases the activity of the phosphodiesterase.

18. The method of claim 15, wherein the phosphodiesterase is PDE4D2.

19. A method of identifying a phosphodiesterase 4D2 (PDE4D2) ligand that selectively binds a phosphodiesterase 4D2 (PDE4D2) polypeptide compared to other polypeptides, the method comprising:
a) providing atomic coordinates of a phosphodiesterase 4D2 (PDE4D2) catalytic domain in complex with a ligand to a computerized modeling system; and
b) modeling a ligand that fits into the binding pocket of a phosphodiesterase 4D2 (PDE4D2) catalytic domain and that interacts with residues of a phosphodiesterase 4D2 (PDE4D2) catalytic domain that are conserved among phosphodiesterase 4D2 (PDE4D2) subtypes to thereby identify a phosphodiesterase 4D2 (PDE4D2) ligand that selectively binds a phosphodiesterase 4D2 (PDE4D2) polypeptide compared to other polypeptides.

20. The method of claim 19, wherein the phosphodiesterase 4D2 (PDE4D2) catalytic domain comprises the amino acid sequence shown in SEQ ID NO: 4.

21. The method of claim 19, further comprising identifying in a biological assay for phosphodiesterase 4D2 (PDE4D2) activity a modeled ligand that selectively binds to said phosphodiesterase 4D2 (PDE4D2) and increases or decreases the activity of the phosphodiesterase 4D2 (PDE4D2).

22. A method for designing a ligand of a phosphodiesterase 4D2 (PDE4D2) polypeptide, the method comprising:
a) forming a complex of a compound bound to the phosphodiesterase 4D2 (PDE4D2) polypeptide;
b) determining a structural feature of the complex formed in (a);
wherein the structural feature is of a binding site for the compound; and
c) using the structural feature determined in (b) to design a ligand of a phosphodiesterase 4D2 (PDE4D2) polypeptide capable of binding to the binding site of claim 7.

23. The method of claim 22, further comprising using a computer-based model of the complex formed in (a) in designing the ligand.

24. A method of designing a chemical compound that modulates the biological activity of a target phosphodiesterase polypeptide, the method comprising:
a) obtaining three-dimensional structures for a catalytic domain (CD) of phosphodiesterase 4D2 (PDE4D2) bound to a ligand, wherein the structures are selected from the group consisting of X-ray structures and computer generated models;
b) rotating and translating the three-dimensional structures as rigid bodies so as to superimpose corresponding backbone atoms of a core region of the phosphodiesterase 4D2 (PDE4D2) CD;
c) comparing the superimposed three-dimensional structures to identify volume near a catalytic pocket of the PDE CD that is available to a ligand in one or more structures, but not available to the ligand in one or more other structures;
d) designing a chemical compound that could occupy the volume in some of the complexed structures, but not in others;
e) synthesizing the designed chemical compound; and
f) testing the designed chemical compound in a biological assay to determine whether it acts as a ligand of a phosphodiesterase with a desired effect on phosphodiesterase biological activities, whereby a ligand of a phosphodiesterase polypeptide is designed.

25. The method of claim 24, further comprising designing a chemical compound by considering a known ligand of the PDE CD and adding a substituent that protrudes into the volume identified in step (c) or that makes a desired interaction.

26. The method of claim 24, wherein the phosphodiesterase is PDE4D2.

27. The method of claim 24, wherein the designing a chemical compound further comprises using computer modeling software.

28. A method of designing a ligand that selectively modulates the activity of a phosphodiesterase polypeptide, the method comprising:
a) evaluating a three-dimensional structure of a crystallized phosphodiesterase 4D2 (PDE4D2) catalytic domain polypeptide in complex with a ligand; and
b) synthesizing a potential ligand based on the three-dimensional structure of the crystallized phosphodiesterase 4D2 (PDE4D2) catalytic polypeptide in complex with a ligand, whereby a ligand that selectively modulates the activity of a phosphodiesterase polypeptide is designed.

29. The method of claim 28, wherein the phosphodiesterase is phosphodiesterase 4D2 (PDE4D2).

30. The method of claim 29, wherein the phosphodiesterase 4D2 (PDE4D2) catalytic domain polypeptide comprises the amino acid sequence of SEQ ID NO: 4.

31. The method of claim 28, wherein the method further comprises contacting a phosphodiesterase catalytic domain polypeptide with the potential ligand and a ligand; and assaying the phosphodiesterase catalytic domain polypeptide for binding of the potential ligand, for a change in activity of the phosphodiesterase catalytic domain polypeptide, or both.

32. A crystallized, recombinant polypeptide comprising: (a) an amino acid sequence set forth in SEQ ID NO: 2 or SEQ ID NO: 4; (b) an amino acid sequence having at least about 95% identity with the amino acid sequence set forth in SEQ ID NO: 2 or SEQ ID NO: 4; or (c) an amino acid sequence encoded by a polynucleotide that hybridizes under stringent conditions to the complementary strand of a polynucleotide having SEQ ID NO: 1 or SEQ ID NO: 3 and has at least one biological activity of PDE4D2; wherein the polypeptide of (a), (b) or (c) is in crystal form.

33. A crystallized complex comprising the crystallized, recombinant polypeptide of claim 32 and a co-factor, wherein the complex is in crystal form.

34. A crystallized complex comprising the crystallized, recombinant polypeptide of claim 32 and a small organic molecule, wherein the complex is in crystal form.

35. The crystallized, recombinant polypeptide of claim 32, which diffracts x-rays to a resolution of about 3.5 Å or better.

36. The crystallized, recombinant polypeptide of claim 32, wherein the polypeptide comprises at least one heavy atom label.

37. The crystallized, recombinant polypeptide of claim 36, wherein the polypeptide is labeled with seleno-methionine.

38. A method for designing a modulator for the prevention or treatment of a disease or disorder, comprising:
a) providing a three-dimensional structure for a crystallized, recombinant polypeptide of claim 32;
b) identifying a potential modulator for the prevention or treatment of a disease or disorder by reference to the three-dimensional structure;
c) contacting a polypeptide of the composition of claim 32 or a phosphodiesterase (PDE) with the potential modulator; and
d) assaying the activity of the polypeptide after contact with the modulator, wherein a change in the activity of the polypeptide indicates that the modulator may be useful for prevention or treatment of a disease or disorder.

39. A method for obtaining structural information of a crystallized polypeptide, the method comprising:
a) crystallizing a recombinant polypeptide, wherein the polypeptide comprises: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; and wherein the crystallized polypeptide is capable of diffracting X-rays to a resolution of 3.5 Å or better; and
b) analyzing the crystallized polypeptide by X-ray diffraction to determine the three-dimensional structure of at least a portion of the crystallized polypeptide.

40. A method for identifying a druggable region of a polypeptide, the method comprising:
a) obtaining crystals of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2, such that the three dimensional structure of the crystallized polypeptide may be determined to a resolution of 3.5 Å or better;
b) determining a three dimensional structure of the crystallized polypeptide using X-ray diffraction; and
c) identifying a druggable region of the crystallized polypeptide based on the three-dimensional structure of the crystallized polypeptide.

41. The method of claim 40, wherein the druggable region is an active site.

42. The method of claim 41, wherein the druggable region is on the surface of the polypeptide.

43. Crystalline human PDE4D2 comprising a crystal having unit cell dimensions a=99.2 Å; b=111.2 Å; c=159.7 Å, α=β=γ=90°, with an orthorhombic space group P2₁2₁2₁, and 4 molecules per asymmetric unit.

44. A crystallized polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; wherein the crystal has a unit cell dimensions a=99.2 Å; b=111.2 Å; c=159.7 Å, α=β=γ=90°, a P2₁2₁2₁, space group, and 4 molecules per asymmetric unit.

45. A crystallized polypeptide comprising a structure of a polypeptide that is defined by a substantial portion of the atomic coordinates set forth in Table 4 or Table 5.

46. A method for determining the crystal structure of a homolog of a polypeptide, the method comprising:
a) providing the three dimensional structure of a first crystallized polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2;
b) obtaining crystals of a second polypeptide comprising an amino acid sequence that is at least 70% identical to the amino acid sequence set forth in SEQ ID NO: 2 or SEQ ID NO: 4, such that the three dimensional structure of the second crystallized polypeptide may be determined to a resolution of 3.5 Å or better; and
c) determining the three dimensional structure of the second crystallized polypeptide by x-ray crystallography based on the atomic coordinates of the three dimensional structure provided in step (a).

47. A method for homology modeling a homolog of human PDE4D2, comprising:
a) aligning the amino acid sequence of a homolog of human PDE4D2 with an amino acid sequence of SEQ ID NO: 2 or SEQ ID NO: 4 and incorporating the sequence of the homolog of human PDE4D2 into a model of human PDE4D2 derived from structure coordinates as listed in Table 4 or Table 5 to yield a preliminary model of the homolog of human PDE4D2;
b) subjecting the preliminary model to energy minimization to yield an energy minimized model;
c) remodeling regions of the energy minimized model where stereochemistry restraints are violated to yield a final model of the homolog of human PDE4D2.

48. A method for obtaining structural information about a molecule or a molecular complex of unknown structure comprising:
a) crystallizing the molecule or molecular complex;
b) generating an x-ray diffraction pattern from the crystallized molecule or molecular complex;
c) applying at least a portion of the structure coordinates set forth in Table 4 or Table 5 to the x-ray diffraction pattern to generate a three-dimensional electron density map of at least a portion of the molecule or molecular complex whose structure is unknown.

49. A method for attempting to make a crystallized complex comprising a polypeptide and a modulator having a molecular weight of less than 5 kDa, the method comprising:
a) crystallizing a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO:
or SEQ ID NO: 3 and has at least one biological activity of human PDE4D2; such that crystals of the crystallized polypeptide will diffract x-rays to a resolution of 5 Å or better; and
b) soaking the crystals in a solution comprising a potential modulator having a molecular weight of less than 5 kDa.

50. A method for incorporating a potential modulator in a crystal of a polypeptide, comprising placing a hexagonal crystal of human PDE4D2 having unit cell dimensions a=99.2 Å; b=111.2 Å; c=159.7 Å, α=β=γ=90°, with an orthorhombic space group P2₁2₁2₁, in a solution comprising the potential modulator.

51. A computer readable storage medium comprising digitally encoded structural data, wherein the data comprises structural coordinates as listed in Table 4 or Table 5 for the backbone atoms of at least about six amino acid residues from a druggable region of human PDE4D2.

52. A scalable three-dimensional configuration of points, at least a portion of the points derived from some or all of the structure coordinates as listed in Table 4 or Table 5 for a plurality of amino acid residues from a druggable region of human PDE4D2.

53. A scalable three-dimensional configuration of points, comprising points having a root mean square deviation of less than about 1.5 Å from the three dimensional coordinates as listed in Table 4 or Table 5 for the backbone atoms of at least five amino acid residues, wherein the five amino acid residues are from a druggable region of human PDE4D2.

54. The scalable three-dimensional configuration of points of claim 53, wherein any point-to-point distance, calculated from the three dimensional coordinates as listed in Table 4 or Table 5, between one of the backbone atoms for one of the five amino acid residues and another backbone atom of a different one of the five amino acid residues is not more than about 10 Å.

55. A scalable three-dimensional configuration of points comprising points having a root mean square deviation of less than about 1.5 Å from the three dimensional coordinates as listed in Table 4 or Table 5 for the atoms of the amino acid residues from any of the above-described druggable regions of human PDE4D2.

56. A computer readable storage medium comprising digitally encoded structural data, wherein the data comprise the identity and three-dimensional coordinates as listed in Table 4 or Table 5 for the atoms of the amino acid residues from any of the above-described druggable regions of human PDE4D2.

57. A scalable three-dimensional configuration of points, wherein the points have a root mean square deviation of less than about 1.5 Å from the three dimensional coordinates as listed in Table 4 or Table 5 for the atoms of the amino acid residues from any of the above-described druggable regions of human PDE4D2, wherein up to one amino acid residue in each of the regions may have a conservative substitution thereof.

58. A scalable three-dimensional configuration of points derived from a druggable region of a polypeptide, wherein the points have a root mean square deviation of less than about 1.5 Å from the three dimensional coordinates as listed in Table 4 or Table 5 for the backbone atoms of at least ten amino acid residues that participate in the intersubunit contacts of human PDE4D2.

59. A computer-assisted method for identifying an inhibitor of the activity of human PDE4D2, comprising:
a) supplying a computer modeling application with a set of structure coordinates as listed in Table 4 or Table 5 for the atoms of the amino acid residues from any of the above-described druggable regions of human PDE4D2 so as to define part or all of a molecule or complex;
b) supplying the computer modeling application with a set of structure coordinates of a chemical entity; and
c) determining whether the chemical entity is expected to bind to or interfere with the molecule or complex.

60. The method of claim 59, wherein determining whether the chemical entity is expected to bind to or interfere with the molecule or complex comprises performing a fitting operation between the chemical entity and a druggable region of the molecule or complex, followed by computationally analyzing the results of the fitting operation to quantify the association between the chemical entity and the druggable region.

61. The method of claim 59, further comprising screening a library of chemical entities.

62. The method of claim 59, further comprising supplying or synthesizing the potential inhibitor, then assaying the potential inhibitor to determine whether it inhibits PDE4D2 activity.

63. A computer-assisted method for designing an inhibitor of PDE4D2 activity comprising:
a) supplying a computer modeling application with a set of structure coordinates having a root mean square deviation of less than about 1.5 Å from the structure coordinates as listed in Table 4 or Table 5 for the atoms of the amino acid residues from any of the above-described druggable regions of human PDE4D2 so as to define part or all of a molecule or complex;
b) supplying the computer modeling application with a set of structure coordinates for a chemical entity;
c) evaluating the potential binding interactions between the chemical entity and the molecule or complex;
d) structurally modifying the chemical entity to yield a set of structure coordinates for a modified chemical entity; and
e) determining whether the modified chemical entity is an inhibitor expected to bind to or interfere with the molecule or complex, wherein binding to or interfering with the molecule or molecular complex is indicative of potential inhibition of PDE4D2 activity.

64. The method of claim 63, wherein determining whether the modified chemical entity is an inhibitor expected to bind to or interfere with the molecule or complex comprises performing a fitting operation between the chemical entity and the molecule or complex, followed by computationally analyzing the results of the fitting operation to evaluate the association between the chemical entity and the molecule or complex.

65. The method of claim 63, wherein the set of structure coordinates for the chemical entity is obtained from a chemical library.

66. The method of claim 63, further comprising supplying or synthesizing the potential inhibitor, then assaying the potential inhibitor to determine whether it inhibits PDE4D2 activity.

67. A computer-assisted method for designing an inhibitor of PDE4D2 activity de novo comprising:
a) supplying a computer modeling application with a set of three-dimensional coordinates derived from the structure coordinates as listed in Table 4 or Table 5 for the atoms of the amino acid residues from any of the above-described druggable regions of human PDE4D2 so as to define part or all of a molecule or complex;
b) computationally building a chemical entity represented by a set of structure coordinates; and
c) determining whether the chemical entity is an inhibitor expected to bind to or interfere with the molecule or complex, wherein binding to or interfering with the molecule or complex is indicative of potential inhibition of PDE4D2 activity.

68. The method of claim 67, wherein determining whether the chemical entity is an inhibitor expected to bind to or interfere with the molecule or complex comprises performing a fitting operation between the chemical entity and a druggable region of the molecule or complex, followed by computationally analyzing the results of the fitting operation to quantify the association between the chemical entity and the druggable region.

69. The method of claim 67, further comprising supplying or synthesizing the potential inhibitor, then assaying the potential inhibitor to determine whether it inhibits PDE4D2 activity.

70. A method for identifying a potential modulator for the prevention or treatment of a disease or disorder, the method comprising:
a) providing the three dimensional structure of a crystallized polypeptide comprising: (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2;
b) obtaining a potential modulator for the prevention or treatment of a disease or disorder based on the three dimensional structure of the crystallized polypeptide;
c) contacting the potential modulator with a second polypeptide comprising: (i) an amino acid sequence set forth in SEQ ID NO: 2 or SEQ ID NO: 4; (ii) an amino acid sequence having at least about 95% identity with the amino acid sequence set forth in SEQ ID NO: 2 or SEQ ID NO: 4; or (iii) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; which second polypeptide may optionally be the same as the crystallized polypeptide; and
d) assaying the activity of the second polypeptide, wherein a change in the activity of the second polypeptide indicates that the compound may be useful for prevention or treatment of a disease or disorder.

71. A method for designing a candidate modulator for screening for inhibitors of a polypeptide, the method comprising:
a) providing the three dimensional structure of a druggable region of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2; and
b) designing a candidate modulator based on the three dimensional structure of the druggable region of the polypeptide.

72. A method for identifying a potential modulator of a polypeptide from a database, the method comprising:
a) providing the three-dimensional coordinates for a plurality of the amino acids of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2;
b) identifying a druggable region of the polypeptide; and
c) selecting from a database at least one potential modulator comprising three dimensional coordinates which indicate that the modulator may bind or interfere with the druggable region.

73. The method of claim 72, wherein the modulator is a small molecule.

74. A method for preparing a potential modulator of a druggable region contained in a polypeptide, the method comprising:
a) using the atomic coordinates for the backbone atoms of at least about six amino acid residues from a polypeptide of SEQ ID NO: 4, with a±a root mean square deviation from the backbone atoms of the amino acid residues of not more than 1.5 Å, to generate one or more three-dimensional structures of a molecule comprising a druggable region from the polypeptide;
b) employing one or more of the three dimensional structures of the molecule to design or select a potential modulator of the druggable region; and
c) synthesizing or obtaining the modulator.

75. An apparatus for determining whether a compound is a potential modulator of a polypeptide, the apparatus comprising:
a) a memory that comprises:
i) the three dimensional coordinates and identities of at least about fifteen atoms from a druggable region of a polypeptide comprising (1) an amino acid sequence set forth in SEQ ID NO: 2 or SEQ ID NO: 4; (2) an amino acid sequence having at least about 95% identity with the amino acid sequence set forth in SEQ ID NO: 2 or SEQ ID NO: 4; or (3) an amino acid sequence encoded by a polynucleotide that hybridizes under stringent conditions to the complementary strand of a polynucleotide having SEQ ID NO: 1 or SEQ ID NO: 3 and has at least one biological activity of human PDE4D2;
ii) executable instructions; and
b) a processor that is capable of executing instructions to:
i) receive three-dimensional structural information for a candidate modulator;
ii) determine if the three-dimensional structure of the candidate modulator is complementary to the three dimensional coordinates of the atoms from the druggable region; and
iii) output the results of the determination.

76. A method for making an inhibitor of PDE4D2 activity, the method comprising chemically or enzymatically synthesizing a chemical entity to yield an inhibitor of PDE4D2 activity, the chemical entity having been identified during a computer-assisted process comprising supplying a computer modeling application with a set of structure coordinates of a molecule or complex, the molecule or complex comprising at least a portion of at least one druggable region from human PDE4D2; supplying the computer modeling application with a set of structure coordinates of a chemical entity; and determining whether the chemical entity is expected to bind or to interfere with the molecule or complex at a druggable region, wherein binding to or interfering with the molecule or complex is indicative of potential inhibition of PDE4D2 activity.

77. A computer readable storage medium comprising digitally encoded data, wherein the data comprises structural coordinates for a druggable region that is structurally homologous to the structure coordinates as listed in Table 4 or Table 5 for a druggable region of human PDE4D2.

78. A computer readable storage medium comprising digitally encoded structural data, wherein the data comprise a majority of the three-dimensional structure coordinates as listed in Table 4 or Table 5.

79. The computer readable storage medium of claim 78, further comprising the identity of the atoms for the majority of the three-dimensional structure coordinates as listed in Table 4 or Table 5.

80. The computer readable storage medium of claim 78, wherein the data comprise substantially all of the three-dimensional structure coordinates as listed in Table 4 or Table 5.
